(19) United States
(12) Patent Application Publication
(10) Pub. No.: US 2009/0068211 A1
(43) Pub. Date: Mar. 12, 2009

(54) IMMUNOGENICALLY-ENHANCED POLYPEPTIDES AND RELATED METHODS

(76) Inventors: YUAN CHANG, PITTSBURGH, PA (US); PATRICK S. MOORE, PITTSBURGH, PA (US)

Correspondence Address: JESSE A. HIRSHMAN, ESQ., 1722 MURRAY AVENUE, THIRD FLOOR, PITTSBURGH, PA, 15217, US

(21) Appl. No.: 12/191,698

(22) Filed: Aug. 14, 2008

Related U.S. Application Data
(60) Provisional application No. 60/955,898, filed on Aug. 15, 2007.

(51) Int. Cl. A61K 39/00; C07H 21/04; C07K 14/435; C07K 1/06; C12N 15/63

(52) U.S. Cl. 424/185.1; 536/23.5; 530/350; 530/403; 435/320.1

(57) ABSTRACT

Described herein are immunogenically-enhanced polypeptides, such as KSHV LANA1 polypeptides, related methods of eliciting an immune response to the polypeptides and related nucleotide sequences. Also described herein are novel polypeptides capable of inhibiting degradation and/or retarding synthesis of a protein when attached to or incorporated within that protein along with related methods and nucleotide sequences.

### CROSS-REFERENCE TO RELATED APPLICATIONS

This application claims the benefit under 35 U.S.C. §119(e) to U.S. Provisional Patent Application No. 60/955,898, filed on Aug. 15, 2007, which is incorporated herein by reference in its entirety.

### STATEMENT REGARDING FEDERAL FUNDING

The U.S. Government has a paid-up license in this invention and the right in limited circumstances to require the patent owner to license others on reasonable terms as provided for by the terms of R01 CA67391 awarded by the National Institutes of Health.

### BACKGROUND

1. Field of the Invention

The present invention is directed to an immunogenically-enhanced Kaposi sarcoma-associated herpesvirus latency-associated nuclear antigen 1 (“KSHV LANA1”) polypeptide and related methods of eliciting an immune response to KSHV LANA1. Also described herein is a novel polypeptide capable of inhibiting degradation of a protein or retarding synthesis of a protein when attached to or incorporated within that protein.

2. Description of the Related Art

Kaposi's sarcoma (“KS”), a lymphatic endothelial cell tumor, became epidemic among AIDS patients in the United States in the early 1980s. The tumor is caused by co-existing infection with Kaposi sarcoma-associated herpesvirus (“KSHV”), also known as human herpesvirus 8 (“HHV8”), an asymptomatic infection that manifests itself as KS among persons with immunosuppression. With the advent of highly active antiretroviral therapy (“HAART”), rates of KS have dramatically declined and by most measures, this tumor has become remarkably well-controlled in U.S. AIDS patients.

There are several reasons why an effective vaccine against KSHV is needed. Rates of KS have declined in the U.S. since their peak in the late 1980s, but KS is now the most commonly-reported tumor in sub-Saharan countries where a vaccine is the only realistic preventive measure. Endemic African KS frequently appears on feet and hands and, if untreated, not only disseminates but also causes morbidity/mortality, even when localized, due to secondary infections. In poverty-stricken regions of Africa, untreated KS leads directly to fatality and contributes indirectly to mortality through disability of family income earners. In the U.S. specific populations remain at high risk for mortality from KS. KS is a leading post-transplant malignancy and occurs in 0.5% of solid organ transplant patients who have a 40-60% mortality rate after contracting disease with the majority of survivors experiencing loss of the transplanted organs. Finally, while KS is currently well controlled among AIDS patients, resurgence of severe KS can be expected. Drop out of effective cytotoxic lymphocyte (“CTL”) responses is likely to be accelerated among aging AIDS patients despite effective antiretroviral therapy (Aboulafia, D. M. 2005. Kaposi sarcoma flares during effective antiretroviral treatment. AIDS Read 15:190-1). Unfortunately, preliminary clinical investigations suggest that aggressive KS may be already reemerging among AIDS patients with otherwise well-controlled HIV infections. In any case, an alternative to drug therapy is welcome both for its long-term cost savings and because vaccination does not require constant long-term administration of drug. Therefore, studies on the basic biology of KSHV that contribute to an effective vaccine have high public health importance.

In 1993, Kaposi sarcoma-associated herpesvirus was found in a KS tumor by representational difference analysis (“RDA”), a sequence-independent technique coupling subtractive hybridization with PCR amplification (Chang, Y., E. Cesarman, M. S. Pessin, F. Lee, J. Culpepper, D. M. Knowles, and P. S. Moore. 1994. Identification of herpesvirus-like DNA sequences in AIDS-associated Kaposi's sarcoma. Science 265:1865-69). Once viral DNA fragments were identified, a causal link for this virus to KS was rapidly established, its viral genome sequenced and specific diagnostic tests were developed (Sarid, R., S. J. Olsen, and P. S. Moore. 1999. Kaposi's sarcoma-associated herpesvirus: Epidemiology, virology and molecular biology. Adv Virus Res 52:139-232). Even before first reports on this virus were published, we found that sera from KS patients specifically react to a latent nuclear antigen in KSHV-infected cells (Moore, P. S., S. J. Gao, G. Dominguez, E. Cesarman, O. Lungu, D. M. Knowles, R. Garber, P. E. Pellett, D. J. McGeoch, and Y. Chang. 1996. Primary characterization of a herpesvirus agent associated with Kaposi's sarcoma. J Virol 70:549-58). These antibodies are not cross-adsorbed with paraformaldehyde-treated extracts from uninfected B cells. Independent characterization of this antigen by two groups (Gao, S.-J., L. Kingsley, D. R. Hoover, T. J. Spira, C. R. Rinaldo, A. Saah, J. Phair, R. Detels, P. Parry, Y. Chang, and P. S. Moore. 1996. Seroconversion to antibodies against Kaposi's sarcoma-associated herpesvirus-related latent nuclear antigens before the development of Kaposi's sarcoma. New Eng J Med 335:233-241 and Kedes, D. H., E. Operskalski, M. Busch, R. Kohn, J. Flood, and D. Ganem. 1996. The seroepidemiology of human herpesvirus 8 (Kaposi's sarcoma-associated herpesvirus): distribution of infection in KS risk groups and evidence for sexual transmission. Nat Med 2:918-24) led to the description of this latency-associated nuclear antigen 1 (“LANA1”) as a useful serologic marker for infection. Rapid progress was then made by several groups in identifying the KSHV gene encoding LANA1 as ORF73 (Kedes, D. H., M. Lagunoff, R. Renne, and D. Ganem. 1997. Identification of the gene encoding the major latency-associated nuclear antigen of the Kaposi's sarcoma-associated herpesvirus. J. Clin. Invest. 100:2606-2610; Kellam, P., C. Boshoff, D. Whitby, S. Matthews, R. A. Weiss, and S. J. Talbot. 1997. Identification of a major latent nuclear antigen, LNA-1, in the human herpesvirus 8 genome. J Hum Virol 1:19-29 and Rainbow, L., G. M. Platt, G. R. Simpson, R. Sarid, S. J. Gao, H. Stoiber, C. S. Herrington, P. S. Moore, and T. F. Schulz. 1997. The 222- to 234-kilodalton latent nuclear protein (LNA) of Kaposi's sarcoma-associated herpesvirus (human herpesvirus 8) is encoded by orf73 and is a component of the latency-associated nuclear antigen. J Virol 71:5915-21).

LANA1 is a 135 kDa protein that aberrantly migrates on SDS gels as doublet bands of approximately (˜) 222-234 kDa and sometimes referred to herein as a ˜220-230 kDA doublet (Gao, S.-J., L. Kingsley, D. R. Hoover, T. J. Spira, C. R. Rinaldo, A. Saah, J. Phair, R. Detels, P. Parry, Y. Chang, and P. S. Moore. 1996. Seroconversion to antibodies against Kaposi's sarcoma-associated herpesvirus-related latent nuclear antigens before the development of Kaposi's sarcoma. New Eng J Med 335:233-241). The doublet protein pattern results from an alternative transcript truncation due to a non-canonical polyA recognition site in the 5′ portion of the gene (Canham, M., and S. J. Talbot. 2004. A naturally occurring C-terminal truncated isoform of the latent nuclear antigen of Kaposi's sarcoma-associated herpesvirus does not associate with viral episomal DNA. J Gen Virol 85:1363-9). By immunohistochemical or immunofluorescent analysis, LANA1 has a speckled nuclear appearance reflecting its association with heterochromatin in interphase cells (Szekely, L., C. Kiss, K. Mattsson, E. Kashuba, K. Pokrovskaja, A. Juhasz, P. Holmvall, and G. Klein. 1999. Human herpesvirus-8-encoded LNA-1 accumulates in heterochromatin—associated nuclear bodies. J Gen Virol 80:2889-900). LANA1 functions as a viral latency origin protein that tethers the KSHV episome to the host chromatin during mitosis (Ballestas, M. E., P. A. Chatis, and K. M. Kaye. 1999. Efficient persistence of extrachromosomal KSHV DNA mediated by latency—associated nuclear antigen. Science 284:641-4). It is constitutively expressed in all KSHV-infected cells.

LANA1 has pleiotrophic functions. In addition to its role in viral persistence and maintenance, LANA1 interacts with a variety of cellular proteins implicated in transformation pathways. Evidence for LANA1 playing a role in oncogenesis comes from studies demonstrating direct interaction between the C-terminal portions of LANA1 and p53 (FIG. 1, prior art) (Friborg, J., Jr., W. Kong, M. O. Hottiger, and G. J. Nabel. 1999. p53 inhibition by the LANA protein of KSHV protects against cell death. Nature 402:889-94). In addition to inhibiting p53-mediated apoptosis, LANA1 interacts with the G1/S checkpoint protein pRB1 but not the related nuclear phosphoproteins p107 and p130, providing an intriguing link to KSHV-related cell proliferation (Radkov, S. A., P. Kellam, and C. Boshoff. 2000. The latent nuclear antigen of kaposi sarcoma-associated herpesvirus targets the retinoblastoma-E2F pathway and with the oncogene hras transforms primary rat cells. Nat Med 6:1121-7). This is functionally similar to the pRB and p53 inhibition properties of SV40 LT.

LANA1 also affects other cell signaling pathways that may contribute to tumorigenesis. Yeast two-hybrid screens show that a LANA1 N-terminal fragment interacts with and inhibits the Wnt signaling inhibitory regulator, glycogen synthetase kinase 3 beta (GSK-3β). In cells, GSK-3β acts to prevent nuclear shuttling of β-catenin, an important positive regulator of the cMYC oncoprotein (Fujimuro, M., and S. D. Hayward. 2003. The latency-associated nuclear antigen of Kaposi's sarcoma-associated herpesvirus manipulates the activity of glycogen synthase kinase-3beta. J Virol 77:8019-30). Expression of LANA1 in heterologous cells efficiently targets GSK-3β, which may contribute to mitogenic signaling in latently infected cells. Further, LANA1 activates transcriptional activity of the Sp1 transcription factor, one consequence of which is increased expression of hTERT (Verma, S. C., S. Borah, and E. S. Robertson. 2004. Latency-Associated Nuclear Antigen of Kaposi's Sarcoma-Associated Herpesvirus Up-Regulates Transcription of Human Telomerase Reverse Transcriptase Promoter through Interaction with Transcription Factor Sp1. J Virol 78:10348-59). At a molecular level, it is probable that LANA1 has pluripotential contributions to KSHV-related tumorigenesis.

Despite these findings, there is no direct evidence that primary effusion lymphoma (“PEL”) cells are dependent on KSHV for survival and transformation (However, see Corte-Real et al. below). Inhibition of LANA1 RNA transcript by siRNA, for example, fails to kill PEL cells (Godfrey, A., J. Anderson, A. Papanastasiou, Y. Takeuchi, and C. Boshoff. 2005. Inhibiting primary effusion lymphoma by lentiviral vectors encoding short hairpin RNA. Blood 105:2510-8). In preliminary results, we showed that LANA1 is required for PEL cell survival using intrabody inhibition of LANA1 (Corte-Real, S., C. Collins, F. Aires da Silva, J. P. Simas, C. Barbas, Y. Chang, P. S. Moore, and J. Goncalves. 2005. Intrabodies targeting the Kaposi's sarcoma-associated herpesvirus latency antigen inhibit viral persistence in lymphoma cells. Corte-Real S, Collins C, Aires da Silva F, Simas J P, Barbas C F 3rd, Chang Y, Moore P, Goncalves J. Intrabodies targeting the Kaposi sarcoma-associated herpesvirus latency antigen inhibit viral persistence in lymphoma cells. Blood. 2005 Dec. 1; 106(12):3797-802. Epub 2005 Aug. 9).

KSHV infected AIDS patients develop antibody responses to LANA1 and other viral proteins—the basis for existing serologic assays. Despite this robust humoral response, neither KSHV infection nor tumorigenesis is controlled. In fact, some AIDS-KS patients have been found with antibody titers against LANA1 greater than 1:150,000 (Gao, S. J., L. Kingsley, M. L1, W. Zheng, C. Parravicini, J. Ziegler, R. Newton, C. R. Rinaldo, A. Saah, J. Phair, R. Detels, Y. Chang, and P. S. Moore. 1996. KSHV antibodies among Americans, Italians and Ugandans with and without Kaposi's sarcoma. Nat Med 2:925-8 and Sitas, F., H. Carrara, V. Beral, R. Newton, G. Reeves, D. Bull, U. Jentsch, R. Pacella-Norman, D. Bourboulia, D. Whitby, C. Boshoff, R. Weiss, M. Patel, P. Ruff, W. R. Bezwoda, E. Retter, and M. Hale. 1999. Antibodies against Human Herpesvirus 8 in Black South African Patients with Cancer. N Engl J Med 340:1863-1871). Clinical evidence from individuals with AIDS, however, reveals the importance of cell-mediated immunity (“CMI”) in controlling KS. Among KSHV-infected persons, CTL responses to both latent and lytic KSHV antigens are present and are presumably responsible for suppression of clinical disease (Gill, J., D. Bourboulia, J. Wilkinson, P. Hayes, A. Cope, A. G. Marcelin, V. Calvez, F. Gotch, C. Boshoff, and B. Gazzard. 2002. Prospective study of the effects of antiretroviral therapy on Kaposi sarcoma—associated herpesvirus infection in patients with and without Kaposi sarcoma. J Acquir Immune Defic Syndr 31:384-90; Osman, M., T. Kubo, J. Gill, F. Neipel, M. Becker, G. Smith, R. Weiss, B. Gazzard, C. Boshoff, and F. Gotch. 1999. Identification of human herpesvirus 8-specific cytotoxic T-cell responses. J Virol 73:6136-40; Wang, Q. J., X. L. Huang, G. Rappocciolo, F. J. Jenkins, W. H. Hildebrand, Z. Fan, E. K. Thomas, and C. R. Rinaldo, Jr. 2002. Identification of an HLA A*0201-restricted CD8(⁺) T-cell epitope for the glycoprotein B homolog of human herpesvirus 8. Blood 99:3360-3366; Wang, Q. J., F. J. Jenkins, L. P. Jacobson, L. A. Kingsley, R. D. Day, Z. W. Zhang, Y. X. Meng, P. E. Pellet, K. G. Kousoulas, A. Baghian, and C. R. Rinaldo, Jr. 2001. Primary human herpesvirus 8 infection generates a broadly specific CD8(⁺) T-cell response to viral lytic cycle proteins. Blood 97:2366-73; Wang, Q. J., F. J. Jenkins, L. P. Jacobson, Y. X. Meng, P. E. Pellett, L. A. Kingsley, K. G. Kousoulas, A. Baghian, and C. R. Rinaldo Jr. 2000. CD8⁺ cytotoxic T lymphocyte responses to lytic proteins of human herpes virus 8 in human immunodeficiency virus type 1-infected and -uninfected individuals. J Infect Dis 182:928-32 and Wilkinson, J., A. Cope, J. Gill, D. Bourboulia, P. Hayes, N. Imami, T. Kubo, A. Marcelin, V. Calvez, R. Weiss, B. Gazzard, C. Boshoff, and F. Gotch. 2002. Identification of Kaposi's sarcoma-associated herpesvirus (KSHV)-specific cytotoxic T-lymphocyte epitopes and evaluation of reconstitution of KSHV-specific responses in human immunodeficiency virus type 1-Infected patients receiving highly active antiretroviral therapy. J Virol 76:2634-40).

CD8⁺ T cell epitopes are generated through endogenous proteasomal degradation and peptidase trimming of viral proteins into 9-mer peptides presented on the surface of antigen presenting cells (APC) by MHC Class I molecules. Each MHC I allele has differing peptide-binding affinity within the presentation cleft resulting in variable epitope presentation and recognition that are thus dependent on the MHC haplotype. CD8⁺ viral peptide epitopes for a given antigen differ depending on the HLA-type of the individual. Structural analyses and peptide-binding studies have generated databases of likely peptide affinities and matrix-based algorithms can be used to predict CTL epitopes for a given viral antigen and MHC I allele-type (Standifer, N., C. Panagiotopoulos, and R. Tan. 2005. Current approaches for the prediction and identification of CD8 T-cell epitopes in type 1 diabetes. Curr Opin Endocrinol Diabetes 12:298-302).

Immune peptide processing does not solely occur with turnover of mature proteins. Up to 30% of nascent peptide chains are misfolded and directly recognized within the endoplasmic reticulum (ER) as defective ribosomal products (DRiPs) (Schubert, U., L. C. Anton, J. Gibbs, C. C. Norbury, J. W. Yewdell, and J. R. Bennink. 2000. Rapid degradation of a large fraction of newly synthesized proteins by proteasomes. Nature 404:770-4). DRiPs are immediately degraded by proteasomes into potential CD8⁺ antigens. Thus, surveillance for foreign antigens through MHC I occurs at both the birth and death of proteins through two independent but related proteolytic processes. In contrast, CD4⁺ T cells survey MHC II complexes for larger peptide fragments generated by lysosomal degradation and exogenous APC presentation. Effective antibody responses and class switching are dependent on CD4⁺-APC interaction and CD4⁺ T cell activation likely accounting for the loss of KSHV seroreactivity among patients infected with KSHV during late-stage AIDS (Gao, S.-J., L. Kingsley, D. R. Hoover, T. J. Spira, C. R. Rinaldo, A. Saah, J. Phair, R. Detels, P. Parry, Y. Chang, and P. S. Moore. 1996. Seroconversion to antibodies against Kaposi's sarcoma-associated herpesvirus-related latent nuclear antigens before the development of Kaposi's sarcoma. New Eng J Med 335:233-241 and Renwick, N., G. J. Weverling, T. Schulz, and J. Goudsmit. 2001. Timing of human immunodeficiency virus type 1 and human herpesvirus 8 infections and length of the Kaposi's sarcoma-free period in coinfected persons. J Infect Dis 183:1427).

KSHV employs several mechanisms to avoid CTL immune surveillance. One method is viral latency itself. The vast bulk of KS tumor cells (>95%) harbor latent virus in which lytic CTL antigen targets are not expressed thus preventing the generation of effective immune responses against the many antigen epitopes in viral proteins that are required for virus replication. Secondly, KSHV encodes multiple chemokines (vMIP-I, -II, and -III) and cytokines (vIL-6) that polarize local immune responses toward Th2 CD4⁺ rather than Th1 CD4⁺ immunophenotypes (Lindow, M., A. Nansen, C. Bartholdy, A. Stryhn, N. J. Hansen, T. P. Boesen, T. N. Wells, T. W. Schwartz, and A. R. Thomsen. 2003. The virus-encoded chemokine vMIP-II inhibits virus-induced Tc1-driven inflammation. J Virol 77:7393-400). These immune modulators are primarily expressed during lytic replication, and would be expected to enhance humoral responses at the expense of cellular immune responses. This may account for the extraordinarily high KSHV antibody titers found in some KS patients. Finally, KSHV encodes two ubiquitin E3 ligases, MIR1 and MIR2, that downregulate surface MHC I and immune co-receptor expression. Cell culture expression studies demonstrate that MIR1 and MIR2 effectively inhibit foreign peptide presentation critical for effective specific CTL immunity (Coscoy, L., and D. Ganem. 2003. PHD domains and E3 ubiquitin ligases: viruses make the connection. Trends Cell Biol 13:7-12). Although MIR1 and MIR2 appear to be expressed primarily during lytic replication, similar downregulation can occur during latent replication (Tomescu, C., W. K. Law, and D. H. Kedes. 2003. Surface downregulation of major histocompatibility complex class I, PE-CAM, and ICAM-1 following de novo infection of endothelial cells with Kaposi's sarcoma-associated herpesvirus. J Virol 77:9669-84). Our laboratory has found that this may be due to an alternatively spliced MIR2 transcript expressed during latency in primary effusion lymphoma cells (Taylor, J. L., H. N. Bennett, B. A. Snyder, P. S. Moore, and Y. Chang. 2005. Transcriptional analysis of latent and inducible Kaposi's Sarcoma-associated Herpesvirus transcripts in the K4 to K7 region. J Virol 79:15099-106).

In Zaldumbide et al., codons for the ovalbumine eptiope were inserted into a green fluorescent protein (“GFP”) gene to create a GFPₒᵥₐ protein (Zaldumbide, A., et al. 2007. In cis inhibition of antigen processing by the latency-associated nuclear antigen I of Kaposi sarcoma Herpes virus. Molec Immunol 44:1352-60). There, the GFPₒᵥₐ was fused either to LANA1 or to a LANA1 variant in which amino acids 330-880 (recited as amino acids 360-911 in that article) were deleted. Whereas presence of amino acids 330-880 in the central acidic-repeat region of LANA1 blocked presentation of the Ova peptide antigen, deletion of amino acids 330-880 from LANA1 prevented LANA1 from blocking antigen presentation. Although Zaldumbide et al. illustrated that the central repeat (“CR”) region is important in inhibiting production of LANA1, the removal of the entire region from the processed protein could limit the epitopes available for antigen presentation. It is therefore desirable to more finely examine which regions in the CR domain of LANA1 are inhibitory to proteosomal degradation and antigen presentation and to retardation of LANA1 protein production.

### SUMMARY

Described herein are novel properties and portions of the KSHV LANA1 protein that can be exploited in producing an immunogenic composition, such as a vaccine, useful in eliciting cell-mediated immunity to LANA1 and therefore to KSHV. Provided therefore are: an immunogenic composition comprising immunologically-enhanced LANA1 (ieLANA1), a method of making an immunogenic composition comprising ieLANA1, a method of using an immunogenic composition comprising ieLANA1 to vaccinate an individual and a commercial kit for distributing an immunogenic composition comprising ieLANA1 in order to implement the described methods.

In one embodiment, a method is provided of eliciting an immune response to KSHV LANA1 in a subject comprising introducing into a cell of the subject an immunologically-enhanced LANA1 polypeptide for eliciting a CTL immune response to LANA1 (e.g. as shown in FIG. 1). The peptide comprises a LANA1 amino acid sequence comprising one or more LANA1 T-cell epitopes, and wherein the LANA1 amino acid sequence is modified to exhibit increased proteasomal degradation or increased synthesis as compared to wild-type LANA1 protein. In one non-limiting embodiment, the polypeptide does not comprise a portion of a LANA1 central repeat domain having the capacity to inhibit proteasomal degradation of a polypeptide or inhibit translation of a polypeptide attached in frame to that portion of the LANA1 central repeat domain, so that the immunologically-enhanced LANA1 polypeptide exhibits increased proteasomal degradation or increased synthesis as compared to wild-type LANA1 protein. In another non-limiting embodiment, the LANA1 polypeptide comprises the amino acid sequence of SEQ ID NO: 1 in which a portion of a LANA1 central repeat domain having the capacity to inhibit proteasomal degradation of a polypeptide and/or inhibit translation of a polypeptide is modified to decrease the ability of that portion to inhibit proteasomal degradation or translation of a polypeptide. For example and without limitation, wherein the LANA1 polypeptide may comprise the amino acid sequence of SEQ ID NO: 1 in which from about amino acid 330 to about amino acid 938 are deleted. In another embodiment, the LANA1 polypeptide comprises the amino acid of SEQ ID NO: 1 in which from amino acid 330 to amino acid 938 are deleted. In another non-limiting embodiment, the LANA1 polypeptide comprises a LANA1 amino acid sequence in which a portion of one or both of central repeat (CR)2 and CR3 of the LANA1 amino acid sequence is modified to decrease the ability of that portion of one or both of CR2 and CR3 to inhibit proteasomal degradation of a polypeptide. For example: the LANA1 amino acid sequence may be modified such that one or both of CR2 and CR3 are substantially or completely deleted, the LANA1 amino acid sequence comprises the amino acid sequence of SEQ ID NO: 1 in which from about 100 to 509 amino acids of amino acids 428-938 are deleted, including a CR2/CR3 junction, CR2 is substantially or completely deleted or replaced in the LANA1 polypeptide, CR3 is substantially or completely deleted or replaced in the LANA1 polypeptide; or CR2 and CR3 are substantially or completely deleted or replaced in the LANA1 polypeptide.

In another non-limiting embodiment, the LANA1 amino acid sequence comprises the amino acid sequence of SEQ ID NO: 1 in which from amino acid 429 to amino acid 938 are deleted. In yet another non-limiting embodiment, the LANA1 amino acid sequence comprises a LANA1 sequence in which at least about 50%, 75% or 95% of both CR2 and CR3 are deleted.

In a further embodiment of the methods of eliciting an immune response to KSHV LANA1 in a patient, the method comprises obtaining a cell from the patient, transforming the cell with a nucleic acid capable of expressing the immunologically-enhanced LANA1 polypeptide in the cell, and transferring the transformed cell back into the patient thereby eliciting the immune response. The cell may be obtained from Peripheral Blood Lymphocytes or may be a dendritic cell. In an alternate embodiment, the polypeptide is administered parenterally to the patient with a pharmaceutically-acceptable excipient, such as an adjuvant.

In yet another embodiment of the method of eliciting an immune response to KSHV LANA1 in a patient, the method comprises transferring a nucleic acid comprising a gene into a cell of the patient, wherein the gene encodes and expresses an immunologically-enhanced LANA1 polypeptide. The nucleic acid may be transferred in vivo by, e.g., direct injection or viral-mediated transduction, in a pharmaceutically-acceptable carrier, optionally including an adjuvant, or ex vivo by, e.g., common transformation or transduction methods.

In a further embodiment of the method of eliciting an immune response to KSHV LANA1 in a patient, the method comprises introducing into the cells of the patient an immunologically-enhanced LANA1 polypeptide, wherein the polypeptide comprises a LANA1 amino acid sequence comprising one or more LANA1 T-cell epitopes and a protein destabilization domain and the immunologically-enhanced LANA1 polypeptide exhibits increased proteasomal degradation as compared to wild-type LANA1 protein. Non-limiting examples of suitable protein destabilization domains include: D-Box, KEN, PEST, Cyclin A and UFD domain/substrate, attached to the LANA1 sequences (directly or indirectly through a linker or other intervening amino acid sequence) as is appropriate to obtain enhanced proteasome degradation of the LANA1 sequences.

In another embodiment, an immunologically-enhanced LANA1 polypeptide for eliciting a CTL immune response to LANA1 is provided. The LANA1 polypeptide comprises a LANA1 amino acid sequence comprising one or more LANA1 T-cell epitopes, wherein the LANA1 amino acid sequence is modified to exhibit increased proteasomal degradation as compared to wild-type LANA1 protein. Various non-limiting embodiments of the polypeptide are described above in connection with the methods, and/or are described throughout this document. In one non-limiting embodiment, the polypeptide does not comprise a portion of a LANA1 central repeat (“CR”) domain having the capacity to inhibit proteasomal degradation of a polypeptide or inhibit translation of a polypeptide attached in frame to that portion of the LANA1 central repeat domain, so that the immunologically-enhanced LANA1 polypeptide exhibits increased proteasomal degradation as compared to wild-type LANA1 protein. In another non-limiting embodiment, the immunologically-enhanced LANA1 polypeptide comprises a LANA1 amino acid sequence comprising one or more LANA1 T-cell epitopes and a protein destabilization domain and the immunologically-enhanced LANA1 polypeptide exhibits increased proteasomal degradation as compared to wild-type LANA1 protein.

In another non-limiting embodiment, a polypeptide other than full-length LANA1 is provided that, comprising at the N-terminal or C-terminal end of the polypeptide, or inserted within the polypeptide an amino acid sequence obtained from or derived from one of both of a CR2 and CR3 region of a LANA1 central repeat domain and having the capacity to inhibit proteasomal degradation of a polypeptide or inhibit translation of the polypeptide as compared to the same polypeptide without the amino acid sequence. In one example, the polypeptide comprises from about amino acid 330 to from about amino acid 938 of SEQ ID NO: 1. In another example, the polypeptide comprises 50 or more, or from about 50 to 509, consecutive amino acids of amino acids 434-938 of SEQ ID NO: 1. In one non-limiting embodiment, the polypeptide comprises a junction between the CR2 and CR3 regions. The polypeptide may comprise a derivative of the portion of a LANA1 central repeat domain having five or more iterations of one or both of the amino acid sequences QQQDE (SEQ ID NO: 3) and QQQEP (SEQ ID NO: 4) or can be a polypeptide comprising from 50 to 100 consecutive amino acids comprising at least about 50% Q residues. In yet another embodiment, the polypeptide may comprise five or more iterations of the amino acid sequence QQQ motifs separated by one or two amino acids, wherein the one or two amino acids may be selected from D, E and P. The polypeptide may further comprise an amino acid sequence comprising five or more iterations of one of the amino acid motif QELEE (SEQ ID NO: 5) attached to the C-terminal end of the portion of the polypeptide comprising from 50 to 100 consecutive amino acids comprising at least about 50% Q residues. The polypeptide also may comprise, without limitation, at least about 25 consecutive polypeptides from a glycine alanine (GA) repeat region of an EBV EBNA1 protein.

In a further non-limiting embodiment, the polypeptide comprises from about 50 to about 148 consecutive amino acids of amino acids 768 to 916 of SEQ ID NO: 1 connected to the C-terminus of from about 50 to about 340 consecutive amino acids of amino acids 428 to 768 of SEQ ID NO: 1.

In yet another non-limiting embodiment, a method is provided of inhibiting proteasomal degradation of a polypeptide comprising attaching to the N-terminal or C-terminal end of the polypeptide an amino acid sequence obtained from or derived from one or both of a CR2 and CR3 region of a LANA1 central repeat domain and having the capacity to inhibit proteasomal degradation of a polypeptide and inhibit translation of the polypeptide as compared to the same polypeptide without the amino acid sequence. In one non-limiting embodiment, the amino acid sequence is obtained from or derived from both of a CR2 and CR3 region of a LANA1 central repeat domain. For example and without limitation, the polypeptide may comprise amino acids 330-938 of SEQ ID NO: 1 or the polypeptide may comprise at least about 100 consecutive amino acids of amino acids 330-938 of SEQ ID NO: 1. Other useful embodiments of a polypeptide useful in this method are described in the preceding paragraph and throughout the present document.

In yet another embodiment, a method of eliciting an immune response to a protein comprising a synthesis retardation and proteasome degradation inhibition domain in a subject is provided. The method comprises introducing into a cell of the subject an immunologically-enhanced version of the protein for eliciting a CTL immune response to the protein, comprising an amino acid sequence of the protein comprising one or more T-cell epitopes of the protein, and wherein the amino acid sequence of the protein is modified to exhibit increased proteasomal degradation as compared to a wild-type version of the protein. In one non-limiting embodiment, the immunologically-enhanced version of the protein does not comprise a portion of a domain having the capacity to inhibit proteasomal degradation of a polypeptide or inhibit translation of a polypeptide attached in frame to that portion of the protein, so that the immunologically-enhanced version of the protein exhibits increased proteasomal degradation as compared to wild-type LANA1 protein. The protein may be a gammaherpesvirus latency protein, such as, without limitation, an EBV EBNA1 protein. In another embodiment, the immunologically-enhanced version of the protein comprises an amino acid sequence comprising one or more one or more T-cell epitopes of the protein and a protein destabilization domain and the immunologically-enhanced version of the protein exhibits increased proteasomal degradation as compared to a wild-type version of the protein. As above, the protein destabilization domain may be one of a D-Box, KEN, PEST, Cyclin A and UFD domain/substrate. Also provided is a polypeptide comprising an immunologically-enhanced version of a protein comprising a synthesis retardation and proteasome degradation inhibition domain protein for eliciting a CTL immune response to the protein, comprising an amino acid sequence of the protein comprising one or more T-cell epitopes of the protein, and wherein the amino acid sequence of the protein is modified to exhibit increased proteasomal degradation as compared to a wild-type version of the protein. In one non-limiting embodiment, the immunologically-enhanced version of the protein does not comprise a portion of a domain having the capacity to inhibit proteasomal degradation of a polypeptide or inhibit translation of a polypeptide attached in frame to that portion of the protein, so that the immunologically-enhanced version of the protein exhibits increased proteasomal degradation as compared to wild-type protein. The protein may be a gammaherpesvirus latency protein, such as, without limitation, EBV EBNA1. In a non-limiting embodiment, the immunologically-enhanced version of the protein comprises an amino acid sequence comprising one or more one or more T-cell epitopes of the protein and a protein destabilization domain and the immunologically-enhanced version of the protein exhibits increased proteasomal degradation as compared to a wild-type version of the protein.

Any of the polypeptides described herein may be prepared by gene expression. As such nucleic acids encoding and, optionally comprising a gene for expression any of the polypeptides described herein are provided. The nucleic acid optionally comprises vector sequences, such as viral vector sequences, for propagating the nucleic acid in a suitable host cell, for packaging the nucleic acid into a viral transducing particle and/or for otherwise facilitating transfer and propagation of the nucleic acid. In one non-limiting example, an isolated nucleic acid is provided comprising an open reading frame encoding a polypeptide other than full-length LANA1 protein, comprising at the N-terminal or C-terminal end of the polypeptide, or inserted within the polypeptide an amino acid sequence obtained from or derived from one of both of a CR2 and CR3 region of a LANA1 central repeat domain and having the capacity to inhibit proteasomal degradation of a polypeptide or inhibit translation of the polypeptide as compared to the same polypeptide without the amino acid sequence. The nucleic acid typically comprises a gene for expressing the open reading frame. The nucleic acid or gene may be contained in a vector and the vector may be a viral vector. In another non-limiting embodiment, the nucleic acid encodes an immunologically-enhanced LANA1 polypeptide for eliciting a CTL immune response to LANA1, as described above and throughout this document.

In the methods described herein, a polypeptide (for example and without limitation, a protein) or nucleic acid comprising a gene for expressing a protein as described herein may be contacted with a cell by any of a number of methods by which a CTL response can be generated. In one embodiment, a polypeptide or nucleic acid is injected parenterally in a patient (in vivo), for example and without limitation intramuscularly, in order to elicit an immune response to the injected polypeptide or protein product of the nucleic acid. In another example, cells from a patient, for example and without limitation, peripheral blood lymphocytes (“PBL”) or dendritic cells (“DC”), are transformed or transduced ex vivo with the nucleic acid, optionally contained within a viral transducing particle, or a polypeptide is administered ex vivo to the cells. The cells would then be transferred back into the patient to elicit an immune response. In any case, for parenteral administration of any polypeptide or nucleic acid described herein, including proteins and transducing particles, the polypeptide or nucleic acid is administered in a composition comprising a pharmaceutically-acceptable carrier, optionally including an adjuvant.

Ideally, the immunogenically-enhanced LANA1 polypeptide comprises highly immunogenic domains of native LANA1 while lacking domains that would inhibit the efficacy of the ieLANA1, such as the degradation inhibition (“DI”) domain and/or the synthesis retardation (“SR”) domain. Unexpected results were obtained when particular portions of the CR domains of LANA1 were deleted. Whereas the CR2 region of LANA1 primarily comprises the DI domain, the CR2/CR3 junction of LANA1 primarily comprises the SR domain. Taken together, deletion of the CR2 region and/or the CR2/CR3 junction results in more precise targeting of domains that would inhibit the efficacy of ieLANA1 and could produce a more highly immunogenic peptide.

### BRIEF DESCRIPTION OF THE DRAWINGS

FIG. 1 provides an amino acid sequence of KSHV LANA1 (GenBank Accession No. AAC55944, SEQ ID NO: 1).

FIG. 2 provides a nucleotide sequence of KSHV LANA1 (GenBank Accession No. U52064, SEQ ID NO: 2).

FIG. 3 (prior art) shows the overall structure of the KSHV LANA1 protein. Shown are the major domains of LANA1 together with known sites of interaction with cellular proteins and the viral replication origin. Used with permission from Lieberman, P. M., J. Hu, and R. Renne. 2006. KSHV and EBV Maintenance and Replication During Latency. In A. Arvin, A. Mocarski, P. S. Moore, B. Roizman, and R. J. Whitley (ed.), Human Herpesviruses: Biology, Therapy and Immunoprophylaxis, vol. in press. Cambridge University Press, Cambridge.

FIG. 4 provides a non-limiting list of human Class I MHC alleles.

FIG. 5A shows immunoblotting for the ˜222-234 kD LANA1 protein. KS patient sera (left) and seronegative blood donor sera (right) using BC-1 cells and a panel of EBV⁺/KSHV-lymphoid cell lines. Note the reactivity of EBNA1 bands. Prior art, from (Gao, S.-J., L. Kingsley, D. R. Hoover, T. J. Spira, C. R. Rinaldo, A. Saah, J. Phair, R. Detels, P. Parry, Y. Chang, and P. S. Moore. 1996. Seroconversion to antibodies against Kaposi's sarcoma-associated herpesvirus-related latent nuclear antigens before the development of Kaposi's sarcoma. New Eng J Med 335:233-241). FIG. 5B shows Northern blotting for ORF73 mRNA in PEL cell lines. Minimal ORF73 encoding mRNA is constitutively present in PEL cells. The polycistronic latency locus encodes at least two transcripts: a high molecular weight LT1 (ORFs 73, 72 and K13) and a lower molecular weight L2T (ORFs 72 and K13 alone). Both LT1 and LT2 transcripts are constitutively present under latent and lytic conditions whereas only LT1 is detected with a LANA1/ORF73 probe. Prior art, from (Sarid, R., J. S. Wiezorek, P. S. Moore, and Y. Chang. 1999. Characterization and cell cycle regulation of the major Kaposi's sarcoma-associated herpesvirus (human herpesvirus 8) latent genes and their promoter. J Virol 73:1438-46).

FIG. 6 shows immunoprecipitation of LANA1. LANA1 was immunoprecipitated with mouse mAb and detected with rat mAb. Note the 150-180 kDa bands are present detected by both antibodies and, like the ˜222/234 kDa bands, show polymorphic variation between cell lines.

FIGS. 7A-7D show a time course for LANA1 turnover after cycloheximide treatment in BCBL1 cells. FIG. 7A is a LANA1 immunoblot of DMSO treated mock control. FIG. 7B is a LANA1 immunoblot at different time points after 50 μg/ml cycloheximide. Note loss of ˜222/234 kDa bands (also referred to herein as ˜220/230 kDa bands due to their variable electrophoretic mobility) after 12 hours but detectable 150-180 kDa LANA1 bands up to 48 hours. FIGS. 7C and 7D, respectively, show the blots of FIGS. 7A and 7B stripped and reprobed with anti-IRF3 (cellular protein).

FIG. 8 showns [³⁵S]-Methionine pulse-chase labelling and immunoprecipitation of LANA1 protein. Note that LANA1 has a t_{1/2} of ˜24 hours but that there is no difference between turnover of the ˜222-234 kDa and the 150-180 kDa bands.

FIGS. 9A and 9B show that the LANA1 central repeat region inhibits LANA1 proteasomal degradation. FIG. 9A shows cycloheximide-treatment 293 cells expressing LANA1 and ORF45 protein. ORF45 protein shows rapid degradation (t_{1/2}˜1 hr) whereas LANA1 has minimal change over a 4-hour time course. FIG. 9B shows MG132 treatment (+) has no affect on full-length LANA1 (top) but markedly increases steady-state protein levels for the LANA-NC fusion, the N terminus and the C-terminus.

FIG. 10 shows that LANA CR2/CR3 stabilizes heterologous proteins and prevents PEST-mediated proteasomal degradation. Kinetics of protein turnover for a de-stabilized GFP (labeled “dsGRP”, top) and a heterologous fusion protein with CR2/CR3 (bottom, labeled “CR2-CR3/N-dsGFP”) after cycloheximide (“CHX”) treatment. Fusion with CR2/CR3 markedly extends the half-life of dsGFP.

FIG. 11 shows CR2/CR3 inhibition of de-stabilized GFP (“dsGFP”) turnover. Cells transfected with constructs described in connection with the experiments depicted in FIG. 10 were examined for GFP fluorescence at time t=0 and at t=12 hours after cycloheximide (“CHX”) treatment. Note that both cells have similar levels of GFP expression at time 0. By 12 hours, dsGFP is nearly absent (top panels) but the CR2/CR3 fusion protein (bottom panel) is still readily detectable. Merged phase contrast shows cell numbers.

FIGS. 12A and 12B show in vitro translation mapping of LANA1 synthesis retardation domains. C-terminal deletion constructs up to the DEQ-rich CR2 domain are efficiently translated (constructs 1-320/HincII and 1-43/Ac1I). The 1-464/SbfI fragment containing 4 repeats from the CR2 region is synthesized at only 40% of the AcII fragment as measured by densitometry. Further retardation occurs in longer 1-928/BsmBI, 1-980/NruI and full length 1-1162. Construct RNAs for all fragments were pre-measured to ensure equal abundance of transcripts for each translation reaction. FIG. 12A provides an electrophoresis gel of the translation reactions, which were performed using equimolar amounts of T7 pol transcribed mRNA and electrophoresed through an 8% SDS polyacrylamide gel. Positive control luciferase RNA translates into ˜61 kDa product. Negative control is without RNA in in vitro uncoupled translation reaction. FIG. 12B provides a schematic of the constructs.

FIG. 13 shows that the LANA1 synthesis retardation domain is localized to the central repeat region. The N-terminal region (labeled “N”), the C-terminal region (labeled “C”) and the N- and C-terminal fusion protein lacking the central repeat region of 312-938 aa (labeled “NC”) are efficiently synthesized whereas full-length LANA1 protein (labeled “FL”) shows clear evidence of synthesis inhibition.

FIG. 14 shows that CR2/CR3 retards translation of EGFP in both the N-terminal and the C-terminal positions. This in vitro translation compares dsEGFP fusion proteins with either N-terminal LANA or with CR2C3. Both orientations of the 80 kD N-terminal LANA fusion protein, but not the CR2/CR3 fusion protein (arrow at expected size), are expressed.

FIGS. 15A and 15B show the effect of amino acid supplementation on LANA1 fragment synthesis. FIG. 15A: Standard amino acid reaction mixture (Promega). FIG. 15B: Supplementation with D, E and Q in excess (1 mM). In vitro translation was performed on fragments shown in FIG. 10 and [³⁵S]-methionine incorporation quantified using a PhosphorImager. Relative LANA1 protein fragment synthesis is similar when either no amino acid supplementation is used or when DEQ amino acids are added in excess suggesting amino acid concentrations are not rate limiting for LANA1 synthesis.

FIG. 16 shows schematically constructs cloned, sequenced, and for which expression has been confirmed. Green constructs are without naturally-occurring methionines in their sequence length. EGFP(N) denotes LANA1 fusion constructs in which the EGFP is N-terminal to LANA1 fragment and EGFP(C) denotes LANA1 fusion constructs in which the EGFP is C-terminal.

FIG. 17 shows hypothetical results for DRiP analysis showing relative amounts of ribosome-associated full-length LANA1 protein and LANA1ΔSRD. These are expected results if a significant fraction of LANA1ΔSRD is processed as a DRiP whereas full-length LANA1 is resistant to DRiP proteolysis. LANA DRiP can be measured by IP or by fluorescence of fusion protein products.

FIGS. 18A and 18B show in vitro translation of LANA1 fragments. FIG. 18A provides a map of successive LANA1 C-terminal deletion constructs. FIG. 18B shows an SDS-PAGE gel of the uncoupled in vitro translation products using the LANA1 C-terminal deletion RNAs shown in FIG. 18A.

FIGS. 19A and 19B show bar graphs of reactions performed using rabbit reticulocyte lysates and [³⁵S]-methionine FIG. 19A is a bar graph showing Phosphoimager quantification of in vitro translated products from FIG. 18B. FIG. 19B is a bar graph showing commercial reticulocyte lysate mixture supplemented with amino acids D, E and Q (1 mM). Translational retardation occurs in cis and is not dependent on local QED amino acid concentrations.

FIG. 20 illustrates that LANA1 CR2/CR3 fragment retards synthesis in vitro at both the N- and the C-terminal positions. In this Figure, CR2/CR3 LANA1 fragment fused to GFP either at the N- or C-terminus prevents translation of heterologously fused proteins. Control reactions consisting of LANA-N-terminus fused N- or C-terminal to GFP are translated.

FIG. 21 depicts the experiment described below distinguishing cis v. trans inhibition in vitro. Increasing amounts of LANA1.FL RNA (containing CR2/CR3) is added to in vitro translation reaction with LANA1.NC (not containing central repeat region). There is no trans inhibition by LANA1.FL.

FIG. 22 shows the effect of LANA1 fragments on protein synthesis in vivo. 293 cells were transfected with GFP-fused LANA1 CR1, CR2, CR3, CR1-CR2 and CR2/CR3. Constructs were harvest at time points 0, 4, 8, 12, 16 and 27 hours and lysates were quantitatively assayed for GFP using a fluorometer. There are decreased amounts of GFP when fused to the LANA1 CR2/CR3 domain.

FIGS. 23A and 23B show degradation retardation of LANA1 in KSHV-infected B lymphoma cell line, BCP-1. LANA1 half-life (FIG. 23A) is prolonged (˜24 hours) compared to cellular IRF3 protein (FIG. 23B). LANA1 immunoblot at different time points after cycloheximide (50 ug/mL) treatment of BCP-1 cells. There was loss of ˜220/230 kDa bands after 12 hrs but 150-180 kDa LANA1 bands (LANA1 isoforms) were detectable up to 48 hrs.

FIG. 24 shows that the degradation inhibition domain of LANA1 is localized to the CR2 region. Constructs encompassing subregions of the LANA1 central repeat region fused to a green fluorescence protein was expressed in cells and assayed after treatment with CHX. Constructs containing CR2 (442-767aa) inhibits protein degradation in comparison to constructs expressing CR1 (330-428aa), CR3 (769-914 aa), and green fluorescence protein alone.

FIG. 25 diagrams the mechanism for CTL immune evasion by the QED central repeat domain of LANA1 The LANA1 central repeat domain may inhibit peptide processing and MHC-I antigen presentation through two distinct mechanisms: (i) synthesis retardation during initial translation from Orf73 mRNA, in which the QED-containing peptide region slows down protein translation, thereby reducing misfolded protein turnover; and (ii) degradation inhibition, in which mature protein proteosomal processing is inhibited by the QED-containing motifs.

### DETAILED DESCRIPTION

As indicated above, provided herein are: an immunogenic composition comprising immunologically-enhanced LANA1 (ieLANA1), a method of making an immunogenic composition comprising ieLANA1, a method of using an immunogenic composition comprising ieLANA1 to vaccinate an individual and a commercial kit for distributing an immunogenic composition comprising ieLANA1 in order to implement the described methods. Although described herein in the context of LANA1, the methods and compositions described herein are equally applicable to other proteins comprising synthesis retardation and/or degradation inhibitory sequences, including, without limitation, gammaherpesvirus latency proteins, such as, without limitation, EBV EBNA1.

As used herein, the term “vaccine” or other forms thereof refer to an immunogenic composition capable of eliciting an immune response to an antigen in a patient. The terms “vaccinate,” “vaccination” or other forms thereof refer to the act of administering a vaccine to a patient in order to elicit an immune response to an antigen in the patient. As used herein, a vaccine can be a population of cells obtained from a patient, manipulated ex vivo and re-administered to the patient in order to elicit an immune response in the patient. In the methods described herein, a polypeptide (for example and without limitation, a protein) or nucleic acid comprising a gene for expressing a protein as described herein may be contacted with a cell by any of a number of methods by which a CTL response can be generated. In one embodiment, a polypeptide or nucleic acid is injected parenterally in a patient (in vivo), for example and without limitation intramuscularly, in order to elicit an immune response to the injected polypeptide or protein product of the nucleic acid. In another example, cells from a patient, for example and without limitation, peripheral blood lymphocytes (PBL) or dendritic cells (DC), are transformed or transduced ex vivo with the nucleic acid, optionally contained within a viral transducing particle, or a polypeptide is administered ex vivo to the cells. The cells would then be transferred back into the patient to elicit an immune response.

A “patient” refers to a live subject, such as a human subject or an animal subject. A vaccine may be administered for any number of reasons, including, without limitation, to elicit protective immunity (partial or complete, humoral or cellular) or to induce a specific immune cell population for, without limitation, research purposes or for commercial purposes, such as, without limitation, to study the effect of such immunization, to produce specific cell populations in a patient, to produce cell products, including, without limitation, effectors (such as, without limitation, antibodies and cytokines) and other uses. Cell populations and cell products may be used, for example and without limitation, in diagnostic assays, in research or therapeutically, such as, without limitation, in adoptive transfer of cells. Uses or cells or cell products may be syngeneic (including self), allogeneic or xenogeneic.

The term “comprising” in reference to a given element of a method, composition, apparatus, etc., means that the method, composition or apparatus includes that element, but also may contain other non-specified elements.

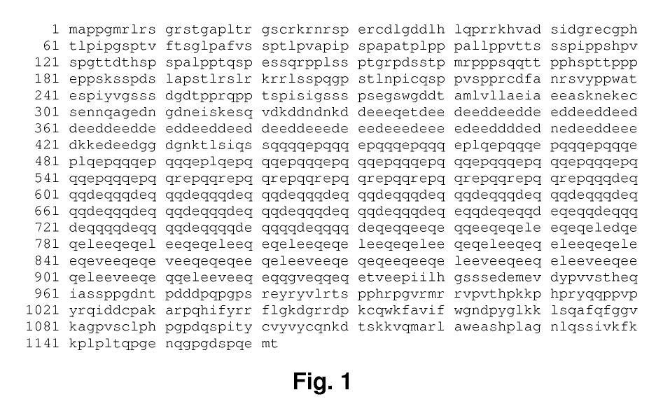

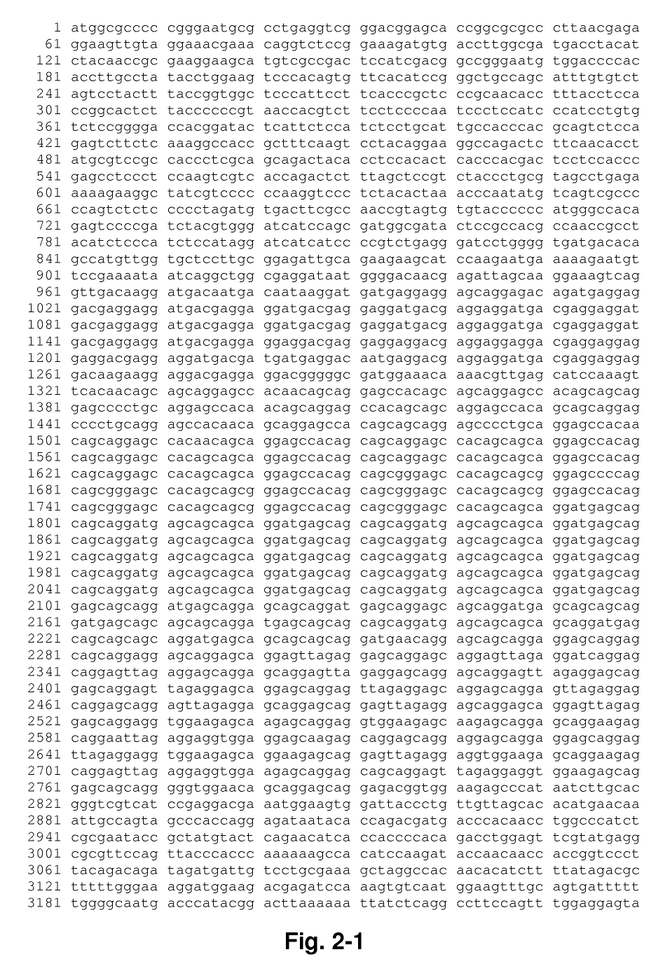

LANA1 (in one embodiment, Genbank Accession Nos. AAC55944 (SEQ ID NO: 1, protein) and U52064 (SEQ ID NO: 2, nucleotide), FIGS. 1 and 2, respectively is one of a few KSHV proteins obligatorily expressed during latent replication. Amino acid residue numbering for LANA1, unless stated otherwise, is in reference to the amino acid sequence provided in FIG. 1 (SEQ ID NO: 1). LANA1 has basic N-terminal (amino acids 1-329) and C-terminal (amino acids 915-1162) domains and an acidic central repeat domain. The central repeat domain can be further divided into three regions, from N- to C-terminal direction: a DED-rich region (amino acids 330-428), a Q-rich region (amino acids 442-767) and a Leucine zipper region (amino acids 769-914). As shown in the experiments below, proteasomal degradation of both LANA1 and a heterologous protein (GFP—Green Fluorescent Protein) is prevented by the presence of the central repeat domain. Proteasomal degradation is central to the production of epitopes and transport and presentation by MHC Class 1 molecules—a central requirement for the typical establishment of antigen-specific cell-mediated immunity.

Provided therefore, according to one embodiment, is an immunogenically-enhanced LANA1 protein (ieLANA1) in which a portion of the central repeat domain is modified to increase proteasomal processing of the protein and thereby increasing the ability of the protein to elicit a CTL response against LANA1, and thus latent and active KSHV infections. Removal of a sufficient number of degradation-inhibitory and/or synthesis (translation) retarding amino acid residues from the LANA1 CR2 and/or CR3 regions to increase proteasomal degradation of LANA1 would produce an ieLANA1 protein. By “immunogenically-enhanced,” it is meant that the ieLANA1 protein is subject to increased proteasomal degradation and, thus is expected to elicit an increased CTL immune response as compared to wild-type (wt) LANA1 protein by virtue of increased presentation by Class 1 MHC chaperones. The LANA1 protein is immunogenically-enhanced by removal of or modification of substantially all of the central repeat (CR) domain, CR2 and/or CR3, or enough of the CR2 and/or CR3 domain, such as, without limitation the CR2/CR3 junction, to increase the ability of the protein to elicit a CTL response against LANA1 as compared to wt LANA1. CR2 and CR3 consist of multiple iterations of certain 3-10 amino acid motifs and a junction region between CR2 and CR3 (for example and without limitation, the CR2/CR3 junction resides between amino acid residues 765 and 775 of SEQ ID NO: 1 or residues 766 and 770 of SEQ ID NO: 1, including for example E768 or Q767-E768-L769). The iterative nature of these repeats as well as the existence of allelic variation in the number of repeats indicate that the identity of the sequences of CR2 and/or CR3 to be removed is of less importance than removing or otherwise mutating a critical number of the repeats and/or disrupting the junction between CR2 and CR3.

In one embodiment, the immunogenically-enhanced LANA1 protein comprises a polypeptide comprising the N-terminal and C-terminal regions of LANA1, in which the central repeat region or CR2 and CR3 is completely or substantially deleted so as to decrease degradation inhibition and/or synthesis retardation attributable to sequences of the central repeat domain. In certain non-limiting embodiments, in reference to SEQ ID NO: 1, amino acids 1-˜312 or amino acids 1-˜330 of LANA1 are attached directly to amino acids ˜939-1162 of LANA1, excluding intervening amino acids (e.g., amino acids 313-938 of LANA 1). In another non-limiting embodiment, the immunogenically-enhanced LANA1 protein comprises amino acids 1-427 of LANA1 (SEQ ID NO: 1) attached directly to amino acids 939-1162 of LANA1, excluding amino acids 428-938 of LANA 1. In a further embodiment, the ieLANA1 is a polypeptide comprising nine or more consecutive amino acids of a LANA1 protein comprising an MHC Class I antigen, wherein the polypeptide excludes proteasomal degradation and/or synthesis retardation amino acid sequence(s), such as the motifs: Q-Ea-Qb-Xaa1-Ec-Xaa2-Xaa3 (SEQ ID NO: 6), QQQQEP (SEQ ID NO: 7), QQQEP (SEQ ID NO: 4), QQQEPL (SEQ ID NO: 8), QEP, QQREP (SEQ ID NO: 9), QQQDE (SEQ ID NO: 3), QEQQDE (SEQ ID NO: 10), QQQQDE (SEQ ID NO: 11), QEQQEE (SEQ ID NO: 12), QEQELED (SEQ ID NO: 13), QEQELEE (SEQ ID NO: 14), QEQEVEE (SEQ ID NO: 15), QELEEVEE (SEQ ID NO: 16) and/or QEEQELEEVEE (SEQ ID NO: 17), as described below.

As used herein, in the context of “an immunologically-enhanced LANA1 polypeptide for eliciting a CTL immune response to LANA1, comprising a LANA1 amino acid sequence comprising one or more LANA1 T-cell epitopes”, the phrase “wherein the polypeptide does not comprise a portion of a LANA1 central repeat domain having the capacity to inhibit proteasomal degradation of a polypeptide or inhibit translation of a polypeptide attached in frame to that portion of the LANA1 central repeat domain, so that the immunologically-enhanced LANA1 polypeptide exhibits increased proteasomal degradation as compared to wild-type LANA1 protein” means that the polypeptide contains one or more Class I MHC epitopes of LANA1, but all or a portion of the central repeat domain is removed or otherwise modified to increase proteasomal degradation of the LANA1 polypeptide as compared to wild-type LANA1. This does not mean that all sequences that contribute to the reduction of proteasomal degradation have to be removed, but either: 1) all sequences that contribute to the reduction of proteasomal degradation are removed or 2) some sequences that contribute to the reduction of proteasomal degradation are removed, leaving some sequences in the polypeptide that retain that capacity. A protein sequence can be “removed,” by modification, such as by insertion of amino acids that disrupt the relevant function, that is, in the context of the present disclosure, proteasomal degradation.

An “MHC Class I antigen” is a polypeptide that is capable of eliciting an TCTL immune response and which comprises an agretope (that part of a processed antigen that binds to the Class I MHC molecule) and an epitope (that part of a processed antigen that binds to a T Cell receptor). In one non-limiting embodiment, the polypeptide is a nonamer that does not require proteasome digestion. In another non-limiting embodiment, the polypeptide is proteasomally-digestable, meaning that it is digestible by a proteasome to produce an MHC Class I antigen. In any case, whether the polypeptide is an MHC Class I antigen or requires proteasomal digestion to yield an MHC Class I antigen, it is useful in eliciting a CTL response by yielding an MHC Class I antigen that can be presented on the surface of a cell.

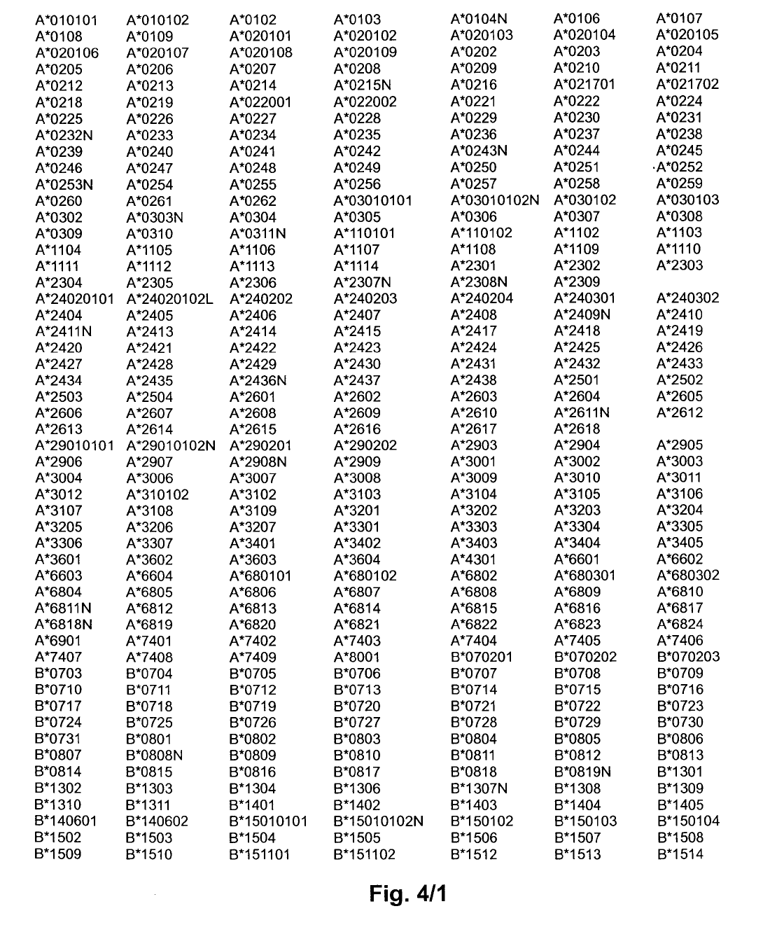

MHC Class I antigens may be determined by using predictive algorithms, for example and without limitation, RANKPEP or the SYFPEITHI database (www.syfpeithi.de). Such predictive algorithms are capable of identifying determinants in the context of many MHC Class I alleles, such as, without limitation, HLA-A0201 (HLA-A2). A large number of MHC class I alleles other than HLA-A0201 also have been identified. Non-limiting examples of MHC class I HLA-A or HLA-B alleles are identified, for example and without limitation, those identified in FIG. 4 (www.anthonynolan.org.uk/HIG/lists/class1list.html). More common Class I MHC alleles include, without limitation: HLA-A1, HLA-A2, HLA-A3, HLA-B7 and HLA-B44. LANA1 Class I MHC antigens (determinants) can be identified for a huge variety or MHC alleles using any of a number of commercially-available software programs, such as those described above. Strings of one or more LANA1 Class I MHC antigens may be strung together in a contiguous engineered polypeptide. The engineered polypeptide may comprise an “epitope string”, an amino acid sequence that contains two or more iterations of any given antigen/epitope, with the object of increasing an immune response to that antigen, for example and without limitation, when that antigen includes a sub-dominant epitope.

One embodiment of the present invention provides a polypeptide capable of eliciting an enhanced CTL response as compared to wild-type LANA1 administered in the same manner. To achieve this, the polypeptide comprises one or more LANA1 MHC Class I antigens (antigenic amino acid sequences that are presentable by class I MHC molecules). The polypeptide, if requiring proteasomal degradation prior to association with Class I MHC, contains proteasomal target residues flanking the antigen and is therefore “preoteasomally digestable.” As is shown herein, certain sequences present in the central repeat domain of LANA1 contribute to resistance to proteasomal degradation of the native LANA1 polypeptide. As such, in order to obtain an ieLANA1, the central repeat domain, or a portion thereof able to inhibit proteasomal degradation of the protein, as compared to wild-type LANA1 should be removed to increase proteasomal degradation. This degradation inhibitory and/or synthesis retardation domain is found, for example and without limitation, between amino acid residues 319 and 938, and more specifically and predominantly between amino acid residues 428-938 of the LANA1 polypeptide, as shown in FIG. 1 (SEQ ID NO: 1). As shown below, the exact residues responsible for the increased resistance of LANA1 to proteasomal degradation are predominantly present within the CR2 and/or CR3 portions of the central repeat domain and can be identified quite readily, as outlined in further detail below. As shown in the experiments below, turnover of LANA1-derived polypeptides is increased by the removal of the Central Repeat Domains and stability of green-fluorescent protein (GFP) is increased (that is, the half-life of the protein is increased) by attachment of the CR2/CR3 domains of LANA1.

In one non-limiting embodiment of the present invention, an ieLANA1 polypeptide is used in a method to elicit a CTL response against LANA1 in a subject, such as a human patient. To successfully elicit (raise) such an immune response, an MHC Class I antigen must be presented by a cell by a class I MHC molecule. In order to do so, the ieLANA1 polypeptide must be introduced into a cell for appropriate processing. By “introduced” it is meant physical introduction of a protein or introduction of a gene into a cell for production of the protein to be introduced. In one embodiment, and perhaps the simplest and safest method, cells, for example and without limitation, peripheral blood lymphocytes (PBLs) or Dendritic Cells (DCs) are obtained from a patient. A gene for expression of the LANA1 polypeptide in lymphocytes is transferred into the cells ex vivo and the cells are transferred back into the patient. In the patient, the cells express the ieLANA1, which is capable of eliciting a CTL response against LANA1 in the patient, thereby eradicating any cells expressing either LANA1 or ieLANA1. Viral vectors or other vectors can be used to transfer a gene in vivo, but the described ex vivo method may be preferred as a method to better control cell transformation and reduce any risk arising from the transfer of recombinant virus directly to a patient, typically by a parenteral route. In vitro or ex vivo, a gene can be transferred into a cell by a number of common methods, including without limitation: viral transduction using a recombinant viral vector incorporated into a viral transducing particle, such a recombinant Adenovirus, Adeno-associated virus, vaccine virus, retrovirus, among others; liposome transfer; electroporation; particle bombardment and calcium phosphate precipitation. In vivo transfer of genetic material to cells can be accomplished using naked DNA, viral transducing particles and liposome preparation as described above and in the art. The in vivo route of delivery can vary so long as an immune response can be generated. It may be preferred to inject DNA, viral transducing particles or liposome preparations locally, such as intra-muscularly, to minimize broad dissemination of the injected material.

In one embodiment, an ieLANA1 protein or a nucleic acid containing a gene encoding the protein can be administered parenterally, such as, without limitation, intramuscularly or subcutaneously, in order to elicit a CTL response against LANA1. In this method, an ieLANA1 is administered in a pharmaceutically-acceptable carrier, which can include an adjuvant and other suitable carriers. The composition administered parenterally comprises an carrier which comprises acceptable excipients, such as, without limitation, one or more suitable: vehicle(s), solvent(s), diluent(s), pH modifier(s), buffer(s), salt(s), colorant(s), rheology modifier(s), lubricant(s), filler(s), antifoaming agent(s), erodeable polymer(s), hydrogel(s), surfactant(s), emulsifier(s), adjuvant(s), preservative(s), phospholipid(s), fatty acid(s), mono-, di- and tri-glyceride(s) and derivatives thereof, wax(es), oil(s) and water, as are broadly known in the pharmaceutical arts. In one embodiment, the carrier is water, in another normal saline and in yet another, phosphate-buffered saline. Adjuvants include, without limitation, Freund's Complete Adjuvant (FCA), Freund's Incomplete Adjuvant (FIA), Montanide ISA Adjuvants (Seppic, Paris, France), Ribi's Adjuvants (Ribi ImmunoChem Research, Inc., Hamilton, Mont.), Gerbu Adjuvant (Gerbu Biotechnik GmbH, Gaiberg, Germany/C-C Biotech, Poway, Calif.), Hunter's TiterMax (CytRx Corp., Norcross, Ga.), aluminum salt adjuvants and nitrocellulose-adsorbed protein.

Irrespective of the method and route of delivery, the treatments are administered as many times, over intervals and for a duration effective to elicit a CTL response against LANA1. In the case of the ex vivo methods, cells transfected, transduced of transformed with a gene for expressing ieLANA1 can be administered intravenously over a time period of minutes, hours, days, etc. The entire batch of cells can be administered to a patient as a single bolus, or in multiple doses over hours, days or weeks. Likewise, cells expressing ieLANA1 can be administered parenterally by routes other than intravenous in any effective dosage regimen.

In one embodiment of the present invention, an amino acid sequence obtained from or derived from one of both of a CR2 and CR3 region and/or a junction therebetween, of a LANA1 central repeat domain and having the capacity to inhibit proteasomal degradation of a polypeptide and/or (“or”) inhibit translation of the polypeptide as compared to the same polypeptide without the amino acid sequence is provided. That amino acid sequence may be used to alter stability of a chimeric protein comprising the LANA1 sequence attached to a second polypeptide. Such a chimeric polypeptide had a multitude of uses, including, without limitation, production of recombinant proteins that are less immunogenic. In one non-limiting embodiment, that amino acid sequence comprises 25 or more consecutive amino acids of amino acids 313-938 of SEQ ID NO: 1. In another embodiment, the amino acid sequence comprises from about 25 to 98 consecutive amino acids of amino acids 330-428 of SEQ ID NO: 1. In another embodiment, a chimeric protein with increased resistance to proteasomal degradation comprises at its N-terminus, C-terminus or internally the motif (Q-Ea-Qb-Xaa1-Ec-Xaa2-Xaa3)n (SEQ ID NO: 6), wherein a is 0 or 1, b is an integer from 1 to 3, c is either 1 or 2, Xaa1 is either R, D or no amino acid, Xaa2 is P or no amino acid, Xaa3 is either L when Xaa2 is P or is no amino acid and n is an integer of at least about 15, in one embodiment, from 25 to 100 in another embodiment from 50 to 75 and in a further embodiment about 70. Each iteration of (Q-Ea-Qb-Xaa1-Ec-Xaa2-Xaa3) (SEQ ID NO: 6) can be the same or different. In one embodiment, a is 1, b is 2 and Xaa1-Ec-Xaa2-Xaa3 is DE or EE. Non-limiting examples of Q-Ea-Qb-Xaa1-Ec-Xaa2-Xaa3 (SEQ ID NO: 6) include QQQQEP (SEQ ID NO: 7), QQQEP (SEQ ID NO: 4), QQQEPL (SEQ ID NO: 8), QEP, QQREP (SEQ ID NO: 9), QQQDE (SEQ ID NO: 3), QEQQDE (SEQ ID NO: 10), QQQQDE (SEQ ID NO: 11) or QEQQEE (SEQ ID NO: 12). In one embodiment, the polypeptide has the sequence of at least 60 contiguous amino acids of residues 428-768 of SEQ ID NO: 1. In another embodiment, the polypeptide has the sequence of at least 60-340 contiguous amino acids of residues 428-768 of SEQ ID NO: 1, inclusive of any integer between 60 and 340; including, without limitation 60, 75, 100, 125, 150, 175, 200, 225, 250, 275, 300, 325 and 340 contiguous amino acids of residues 428-768 of SEQ ID NO: 1.

In yet another embodiment, the polypeptide comprises a sequence having the structure N-A-B-C, in which A comprises two or more independent iterations of 3, 4, 5 or 6 contiguous amino acids of 428-768 of SEQ ID NO: 1 or at least 60-340 contiguous amino acids of residues 428-768 of SEQ ID NO: 1, inclusive of any integer between 60 and 340; including, without limitation 60, 75, 100, 125, 150, 175, 200, 225, 250, 275, 300, 325 and 340 contiguous amino acids of residues 428-768 of SEQ ID NO: 1 and, in one embodiment comprising a junction between the CR2 and CR3 regions. “N” and “C” refer to the N-terminus and C-terminus, respectively. In one non-limiting embodiment, A comprises the sequence (Q-Ea-Qb-Xaa1-Ec-Xaa2-Xaa3)n (SEQ ID NO: 6), wherein a is 0 or 1, b is an integer from 1 to 3, c is either 1 or 2, Xaa1 is either R, D or no amino acid, Xaa2 is P or no amino acid, Xaa3 is either L when Xaa2 is P or is no amino acid and n is an integer of at least about 15, in one embodiment, from 25 to 100 in another embodiment from 50 to 75 and in a further embodiment about 70. Each iteration of (Q-Ea-Qb-Xaa1-Ec-Xaa2-Xaa3) (SEQ ID NO: 6) can be the same or different. In one embodiment, a is 1, b is 2 and Xaa1-Ec-Xaa2-Xaa3 is DE or EE. Non-limiting examples of Q-Ea-Qb-Xaa1-Ec-Xaa2-Xaa3 (SEQ ID NO: 6) include QQQQEP (SEQ ID NO: 7), QQQEP (SEQ ID NO: 4), QQQEPL (SEQ ID NO: 8), QEP, QQREP (SEQ ID NO: 9), QQQDE (SEQ ID NO: 3), QEQQDE (SEQ ID NO: 10), QQQQDE (SEQ ID NO: 11) or QEQQEE (SEQ ID NO: 12).

“B” comprises in one non-limiting embodiment, from 50-148 consecutive amino acids, including any integer therebetween, of residues 768-916 of SEQ ID NO: 1. B can comprise from 2 to 200, from 5-100, from 10-75, from 25-75, inclusive of any integer therebetween, independent iterations of from 7, 8, 9 or 10 contiguous amino acid residues 768-916 of SEQ ID NO: 1. Specific examples of B included two or more independent iterations of one or more of the following amino acid sequences: QEQELED (SEQ ID NO: 13), QEQELEE (SEQ ID NO: 14), QEQEVEE (SEQ ID NO: 15), QELEEVEE (SEQ ID NO: 16) and QEEQELEEVEE (SEQ ID NO: 17). Polypeptide N-A-B-C includes, without limitation, a polypeptide of from 11 to 488 amino acids, inclusive of any integer therebetween having the sequence of from any of residues 428 through 708 to any of residues 818 through 916 of SEQ ID NO: 1. In one non-limiting embodiment of polypeptide N-A-B-C, A is omitted. In another, B is omitted. In either case, proteasome-degradation-inhibitory polypeptide sequences that are identical to native LANA1 sequences, for example and without limitation, as provided in SEQ ID NO: 1, are considered to be “obtained from” a LANA1 sequence. Artificial sequences that contain non-naturally occurring combinations of iterations of the Q-Ea-Qb-Xaa1-Ec-Xaa2-Xaa3 (SEQ ID NO: 6), QQQQEP (SEQ ID NO: 7), QQQEP (SEQ ID NO: 4), QQQEPL (SEQ ID NO: 8), QEP, QQREP (SEQ ID NO: 9), QQQDE (SEQ ID NO: 3), QEQQDE (SEQ ID NO: 10), QQQQDE (SEQ ID NO: 11), QEQQEE (SEQ ID NO: 12), QEQELED (SEQ ID NO: 13), QEQELEE (SEQ ID NO: 14), QEQEVEE (SEQ ID NO: 15), QELEEVEE (SEQ ID NO: 16) and/or QEEQELEEVEE (SEQ ID NO: 17) motifs are sequences “derived from” a LANA1 sequence, though the 3-10 amino acid motifs may be “obtained from” the native protein.

By extrapolating the studies with the GA region of EBNA1 and in light of the known allelic variations in the CR2 and CR3 regions of LANA1, it is expected that many, if not all of the above-described sequences would impart resistance to proteasomal degradation when attached to (C- or N-terminal) or inserted within a given polypeptide sequence. A person of ordinary skill in the art can readily determine the ability of any polypeptide to affect protein turnover in cis, and even affect translation, by conducting, for example and without limitation, the assays described herein, such as by attaching the polypeptide to GFP and determining protein stability in the presence of cycloheximide.

In one embodiment, a nucleic acid containing a sequence, such as an open reading frame (ORF), encoding an ieLANA1 polypeptide is provided. In one embodiment, a nucleic acid sequence encoding any of the above-described ieLANA1 polypeptides is incorporated into a gene for expressing that ieLANA1 polypeptide. In another, a proteasome-degradation-inhibitory polypeptide sequence, as described above, is attached, in-frame, with a nucleic acid encoding a protein to produce an ORF encoding a protein with increased stability against proteasomal degradation. Non-limiting examples of candidates for proteins that could benefit from addition of a proteasome-degradation-inhibitory polypeptide sequence include indicator proteins, such as GFP, enzymes, particularly enzymes for use in vivo, such as, without limitation, adenosine deaminase to treat severe-combined immunodeficiency (SCID) (Mortellaro A, Jofra Hernandez R, Guerrini M M, Carlucci F, Tabucchi A, Ponzoni M, Sanvito F, Doglioni C, Di Serio C, Biasco L, Follenzi A, Naldini L, Bordignon C, Roncarolo M G, Aiuti A. Ex vivo gene therapy with lentiviral vectors rescues adenosine deaminase (ADA)-deficient mice and corrects their immune and metabolic defects. Blood. 2006 Jul. 11; Miseki T, Kawakami H, Natsuizaka M, Darmanin S, Cui H Y, Chen J, Fu Q, Okada F, Shindo M, Higashino F, Asaka M, Hamuro J, Kobayashi M. Suppression of tumor growth by intra-muscular transfer of naked DNA encoding adrenomedullin antagonist. Cancer Gene Ther. 2006 Jul. 14; Ponder K P. Gene therapy for hemophilia. Curr Opin Hematol. 2006 Sep.; 13(5):301-7. PMID: 16888433 and Herrera J L, Femandez-Montesinos R, Gonzalez-Rey E, Delgado M, Pozo D. Protective Role for Plasmid DNA-Mediated VIP Gene Transfer in Non-Obese Diabetic Mice. Ann N Y Acad. Sci. 2006 July; 1070:337-41). As described above, the proteasome-degradation-inhibitory polypeptide sequences can be attached to the N- or C-terminus of the protein, or even internally, so as to best retain the structure or function of the protein. Vectors containing expression cassettes are broadly available for expression of genes in various host cells, such as E. coli, S. cerevisiae, insect and mammalian cells, such as Chinese Hamster Ovary (CHO) cells or human cells. Although DNA consisting only of a gene for expressing a recombinant protein (an ieLANA1 or a protein containing a proteasome-degradation-inhibitory polypeptide sequence) can be used to transfect or transform a cell, a huge number of vector and transformation systems, many of which are well-known and beyond the scope of this disclosure, are useful in producing a cell that expresses a recombinant protein. Some of these vector systems are known, including, without limitation: yeast, insect, bacterial, mammalian and viral (for example, phage, retroviral, Adenoviral, and Adeno-associated virus) vector systems. Suitable vectors, cells and, in general, expression systems are available commercially from a large variety of sources, including without limitation, Stratagene of La Jolla, Calif. and the American Type Culture Collection (ATCC) of Manassass, Va. In another non-limiting example, plasmid- or episome-based systems useful in gene transfer and expression are broadly known. Any gene for expression of a given ieLANA1 polypeptide or protein containing a proteasome-degradation-inhibitory polypeptide sequence can be inserted into a suitable vector for transfer and expression in a cell.

By “expression” it is meant the overall flow of information from a gene (without limitation, a functional genetic unit for producing a gene product in a cell or other expression system encoded on a nucleic acid and comprising: a transcriptional promoter and other cis-acting elements, such as response elements and/or enhancers; an expressed sequence that typically encodes a protein (open-reading frame or ORF) or functional/structural RNA, and a polyadenylation sequence), to produce a gene product (typically a protein, optionally post-translationally modified or a functional/structural RNA). By “expression of genes under transcriptional control of,” or alternately “subject to control by,” a designated sequence, it is meant gene expression from a gene containing the designated sequence operably linked (functionally attached, typically in cis) to the gene. The designated sequence may be all or part of the transcriptional elements (without limitation, promoters, enhancers and response elements), and may wholly or partially regulate and/or affect transcription of a gene. A “gene for expression of” a stated gene product is a gene capable of expressing that stated gene product when placed in a suitable environment—that is, for example, when transformed, transfected, transduced, etc. into a cell, and subjected to suitable conditions for expression. In the case of a constitutive promoter “suitable conditions” means that the gene typically need only be introduced into a host cell. In the case of an inducible promoter, “suitable conditions” means when an amount of the respective inducer is administered to the expression system (e.g., cell) effective to cause expression of the gene.

Any nucleic acid encoding a given polypeptide sequence can be prepared by a variety of known methods. For example and without limitation, by direct synthesis of the primary DNA sequence for insertion in a gene, gene cassette, vector, etc., by PCR cloning methods, or by restriction and ligation or recombination according to well-established practices. In the case of preparation of a nucleic acid sequence encoding a repetitive sequence, a nucleic acid encoding a single iteration of the repeat may be prepared with blunt or sticky ends, as is known in the art, and subsequently ligated to form multiple iterations. The ligated iterative sequences can then be ligated into a vector, gene or gene cassette by known methods. One example of such a single iteration is a sequence encoding the sequence DEQQQ, with codons for D, E and Q, being G-A-U/C, G-A-A/G and C-A-A/G, respectively. Thus example of a suitable single iteration that can be ligated to produce multiple iterations is:

| | |
|---|---|
| CAAGACGAGCAACAA . . . | (SEQ ID NO: 19) |
| CTGCTCGTTGTTGTT | (SEQ ID NO: 20) |

A double iteration can also be used for cloning purposes, for example and without limitation (note alternate codon usage in second iteration—iterations separated by hyphen for illustrative purposes only):

(SEQ ID NO: 21)

CAAGACGAGCAACAACAA-GATGAACAGCAA . . .

(SEQ ID NO: 22)

CTGCTCGTTGTTGTT-CTACTTGTCGTTGTT

With regard to the ieLANA1 polypeptide, and in the context of the methods, compositions, polypeptide sequences and nucleic acid sequences, and other embodiments thereof described herein, a LANA1 polypeptide attached, in-frame to (fused with) a protein destabilization sequence is likely to be useful in enhancing CTL response to the LANA1 polypeptide. While the glycine-alanine repeat (“Gar”) motif inhibits proteasomal processing of EBNA1, this can be overcome through the introduction of strong destabilizing motifs that enforce turnover of EBNA1 heterologous proteins possessing a GAr stabilization domain (Dantuma, N. P., S. Heessen, K. Lindsten, M. Jellne, and M. G. Masucci. 2000. Inhibition of proteasomal degradation by the Gly-Ala repeat of Epstein-Barr virus is influenced by the length of the repeat and the strength of the degradation signal. Proc Natl Acad Sci USA 97:8381-5). As used herein, a “protein destabilization sequence” is a polypeptide sequence that, when fused (attached in-frame either directly or through an intervening sequence, such as a linker sequence) to LANA1 or a portion thereof, or another protein, will contribute to increased proteasomal degradation of that protein as compared to that same protein without the same destabilization sequence. LANA1 or a portion thereof fused to a protein destabilization sequence are useful in the methods described herein and therefore provided are methods of eliciting immune response to LANA1 comprising introducing into a cell of a subject LANA1 or a portion thereof which is fused to a destabilization sequence. As follows, also provided are polypeptides comprising a LANA1-protein destabilization sequence as well as nucleic acids encoding a LANA1-protein destabilization sequence polypeptide and compostions comprising either. In one embodiment, full-length LANA1 is fused to a protein destabilization domain. In another embodiment, from about 50 to 1161 contiguous amino acids, and integers therebetween of LANA1 are fused to a protein destabilization sequence.

There are several strategies to destabilize proteins to enforce their rapid proteasomal turnover. Cell cycle-dependent proteins must undergo rapid and complete ubiquitin-mediated proteolysis to achieve cycling within the cell cycle. Primary structural domains, including, without limitation, the nine residue D-box (Yamano, H., C. Tsurumi, J. Gannon, and T. Hunt. 1998. The role of the destruction box and its neighbouring lysine residues in cyclin B for anaphase ubiquitin-dependent proteolysis in fission yeast: defining the D-box receptor. Embo J 17:5670-8), the KEN box (Pfleger, C. M., and M. W. Kirschner. 2000. The KEN box: an APC recognition signal distinct from the D box targeted by Cdh1. Genes Dev 14:655-65)(63), A-box (Nguyen, H. G., D. Chinnappan, T. Urano, and K. Ravid. 2005. Mechanism of Aurora-B degradation and its dependency on intact KEN and A-boxes: identification of an aneuploidy-promoting property. Mol Cell Biol 25:4977-92) and PEST domains (Fung, T. K., W. Y. Siu, C. H. Yam, A. Lau, and R. Y. Poon. 2002. Cyclin F is degraded during G2-M by mechanisms fundamentally different from other cyclins. J Biol Chem 277:35140-9) have been used in heterologous fusion proteins to initiate rapid protein degradation. Cloning D-box/KEN domains into LANA1 will cause the fusion protein to become an anaphase-promoting complex/cyclosome (APC) E3 ligase target that should result in its rapid turnover. N-end rule proteins (containing an N-end rule substrate or N-degron) and ubiquitin-fusion degradation (UFD) proteins are rapidly processed for proteasomal destruction (Dantuma, N. P., K. Lindsten, R. Glas, M. Jellne, and M. G. Masucci. 2000. Short-lived green fluorescent proteins for quantifying ubiquitin/proteasome-dependent proteolysis in living cells. Nat Biotechnol 18:538-43 and Varshavsky, A. 1996. The N-end rule: functions, mysteries, uses. Proc Natl Acad Sci USA 93:12142-9).

In one embodiment, LANA1 may be destabilized by cloning the N-terminal destruction region of cyclin A in-frame into LANA1 (Kaspar, M., A. Dienemann, C. Schulze, and F. Sprenger. 2001. Mitotic degradation of cyclin A is mediated by multiple and novel destruction signals. Curr Biol 11:685-90). This is a simple modification that encodes multiple destruction sequences, and has been successful in initiating rapid degradation in a variety of proteins that have been modified. It is anticipated that the cyclin A-LANA1 fusion protein will undergo rapid proteolytic degradation that can be determined through the kinetics of [35S]-methionine pulse-chase labeling and immunoprecipitation after transfection into 293 cells.

In another embodiment, as an alternative approach, a UFD substrate containing an N-terminal Ub-R fusion with linker sequence and lysine residue at position 17 can be engineered. This substrate has been shown to be a potent and nonspecific activator of proteasomal degradation (Dantuma, N. P., K. Lindsten, R. Glas, M. Jellne, and M. G. Masucci. 2000. Short-lived green fluorescent proteins for quantifying ubiquitin/proteasome-dependent proteolysis in living cells. Nat Biotechnol 18:538-43) and may be fused to the N-terminus of a LANA1 polypeptide.

In yet another embodiment of the ieLANA1 polypeptide fused with a destabilization sequence, and in the context of the methods, compositions, polypeptide sequences and nucleic acid sequences, and other embodiments thereof described herein, either a dibasic endopeptidase recognition site or a caspase-recognition site can be engineered on either side of the DI domain. In doing so, it is anticipated that the LANA1 protein will be cleaved after transfection into 293 cells, leading to rapid degradation of fragments. The cleavage product(s) lacking the DI domain should undergo rapid proteolysis.

By fusing a protein destabilization sequence, such as, without limitation, D-Box, KEN, PEST, Cyclin A and UFD domains/substrates, to a LANA1 polypeptide, it is expected that LANA1 will be destabilized. Immunoblotting in the presence of proteasome inhibitors may be performed to investigate the mechanism of LANA1 degradation-inhibition. It is assumed that this will generate a polyubiquitinylated protein ladder for the LANA1 fusion protein but not for wild-type LANA1 if the DI domain inhibits ubiquitinylation. If the DI acts to inhibit proteasomal processing downstream from the ubiquitinylation step (such as by preventing proteasomal unfoldase activity), no change in polyubiquitinylation profiles are expected to be seen when cells transfected with the two LANA proteins are treated with proteasome inhibitors. Thus, either through use of destabilizing destruction domains or through endogenous proteolytic cleavage of LANA1, it is anticipated that it will be possible to generate a version of LANA1 protein that is rapidly processed through the cellular proteasome system.

### EXAMPLES

The following addresses the inquiry of whether LANA1 possesses physical features that prevent effective immunoprocessing and anti-LANA1 CTL development. The EBV EBNA1 protein escapes from MHC I-CD8⁺ cell surveillance through two mechanisms. First, through a well-described glycine-alanine repeat (GAr) region, EBNA1 inhibits its own proteasomal processing (Levitskaya, J., A. Sharipo, A. Leonchiks, A. Ciechanover, and M. G. Masucci. 1997. Inhibition of ubiquitin/proteasome-dependent protein degradation by the Gly-Ala repeat domain of the Epstein-Barr virus nuclear antigen 1. Proc Natl Acad Sci USA 94:12616-21). Secondly, the GAr motif acts to retard EBNA1 RNA translation, increasing proper EBNA1 folding and reducing EBNA1 DRiP degradation into potentially antigenic peptides (Yin, Y., B. Manoury, and R. Fahraeus. 2003. Self-inhibition of synthesis and antigen presentation by Epstein-Barr virus-encoded EBNA1. Science 301:1371-4). The EBNA1 GAr was first shown by Levitskaya et al. to directly inhibit proteasomal processing of EBNA1 protein (Levitskaya, J., M. Coram, V. Levitsky, S. Imreh, P. M. Stigerwald-Mullen, G. Klein, M. G. Kurilla, and M. G. Masucci. 1995. Inhibition of antigen processing by the internal repeat region of the Epstein-Barr virus nuclear antigen-1. Nature 375:685-88 and Levitskaya, J., A. Sharipo, A. Leonchiks, A. Ciechanover, and M. G. Masucci. 1997. Inhibition of ubiquitin/proteasome-dependent protein degradation by the Gly-Ala repeat domain of the Epstein-Barr virus nuclear antigen 1. Proc Natl Acad Sci USA 94:12616-21). EBNA1 is structurally stable and has an extraordinarily long half-life (Davenport, M. G., and J. S. Pagano. 1999. Expression of EBNA-1 mRNA is regulated by cell cycle during Epstein-Barr virus type I latency. J Virol 73:3154-61 and Levitskaya, J., A. Sharipo, A. Leonchiks, A. Ciechanover, and M. G. Masucci. 1997. Inhibition of ubiquitin/proteasome-dependent protein degradation by the Gly-Ala repeat domain of the Epstein-Barr virus nuclear antigen 1. Proc Natl Acad Sci USA 94:12616-21). Using a GAr construct fused with IκB, that group demonstrated that the repeat region does not inhibit ubiquitinylation, but rather limits its post-ubiquitin signaling proteasomal processing (Sharipo, A., M. Imreh, A. Leonchiks, S. Imreh, and M. G. Masucci. 1998. A minimal glycine-alanine repeat prevents the interaction of ubiquitinated I kappaB alpha with the proteasome: a new mechanism for selective inhibition of proteolysis. Nat Med 4:939-44). While the precise mechanism for this escape from proteolysis remains to be defined, most investigators describe this effect as specific to the Gly-Ala sequence repeat structure, particularly to the repeat spacing of alanine residues (Sharipo, A., M. Imreh, A. Leonchiks, C. Branden, and M. G. Masucci. 2001. cis-Inhibition of proteasomal degradation by viral repeats: impact of length and amino acid composition. FEBS Lett 499:137-42).

Auto-inhibition of EBNA1 proteasomal degradation poses an interesting problem: why doesn't EBNA1 accumulate dramatically within cells compared to EBNA1ΔGAr? This question was answered by Yin and colleagues who found that the GAr also acts in cis to retard EBNA1 protein translational synthesis (Yin, Y., B. Manoury, and R. Fahraeus. 2003. Self-inhibition of synthesis and antigen presentation by Epstein-Barr virus-encoded EBNA1. Science 301:1371-4). N-terminal tagging of proteins with the Gly-Ala repeat markedly reduces their synthesis rate as well as their proteasomal degradation, while C-terminal fusions only inhibit proteasomal degradation. The ability to separate these two GAr-dependent processes, retarded synthesis and degradation inhibition, allowed investigation of the importance of EBNA1 DRiPs in initiation of a CTL response. Endogenous presentation of EBNA1 peptides as CD8⁺ T cell antigens in B cell lines with differential degradation rates lends support to the notion that EBNA1 DRiPs are primarily responsible for generating an immune response (Lee, S. P., J. M. Brooks, H. Al-Jarrah, W. A. Thomas, T. A. Haigh, G. S. Taylor, S. Humme, A. Schepers, W. Hammerschmidt, J. L. Yates, A. B. Rickinson, and N. W. Blake. 2004. CD8 T cell recognition of endogenously expressed epstein-barr virus nuclear antigen 1. J Exp Med 199:1409-20 and Tellam, J., G. Connolly, K. J. Green, J. J. Miles, D. J. Moss, S. R. Burrows, and R. Khanna. 2004. Endogenous presentation of CD8⁺ T cell epitopes from Epstein-Barr virus-encoded nuclear antigen 1. J Exp Med 199:1421-31). No clear mechanism explains either EBNA1 GAr inhibition of proteolysis or synthesis. Since these phenomena have only been examined for the EBNA1 GAr structure, studies of other viral proteins having similar effects could yield important generalizable insights into protein processing (Basta, S., R. Stoessel, M. Basler, M. van den Broek, and M. Groettrup. 2005. Cross-presentation of the long-lived lymphocytic choriomeningitis virus nucleoprotein does not require neosynthesis and is enhanced via heat shock proteins. J Immunol 175:796-805).

LANA1 has functional similarities to EBNA1 though the two proteins have no direct sequence similarity, namely, both are viral episome maintenance proteins for lymphotrophic gammaherpesviruses (Komatsu, T., M. E. Ballestas, A. J. Barbera, and K. M. Kaye. 2002. The KSHV latency-associated nuclear antigen: a multifunctional protein. Front Biosci 7:d726-30). Both EBNA1 and LANA1 dimerize to viral latent replication origins, and there is structural similarity of origins between the two viruses (Garber, A. C., M. A. Shu, J. Hu, and R. Renne. 2001. DNA binding and modulation of gene expression by the latency-associated nuclear antigen of Kaposi's sarcoma-associated herpesvirus. J Virol 75:7882-92; Hu, J., A. C. Garber, and R. Renne. 2002. The Latency-Associated Nuclear Antigen of Kaposi's Sarcoma-Associated Herpesvirus Supports Latent DNA Replication in Dividing Cells. J Virol 76:11677-87 and Srinivasan, V., T. Komatsu, M. E. Ballestas, and K. M. Kaye. 2004. Definition of sequence requirements for latency-associated nuclear antigen 1 binding to Kaposi's sarcoma-associated herpesvirus DNA. J Virol 78:14033-8). Like EBNA1, LANA1 is highly immunogenic provoking a strong antibody response that is non-neutralizing. LANA1 encodes a highly acidic central repeat region that can be divided into aspartate-glutamate (D-E) and aspartate-glutamate-glutamine (D-E-Q) rich repeat subregions. Allelic loss and gain of LANA1 repeat units, as is seen in the EBNA1 GAr (Patel, G. V., M. G. Masucci, G. Winberg, and G. Klein. 1988), is also seen with different isolates of KSHV (Gao S J, Zhang Y J, Deng J H, Rabkin C S, Flore O, Jenson H B. Molecular polymorphism of Kaposi's sarcoma-associated herpesvirus (Human herpesvirus 8) latent nuclear antigen: evidence for a large repertoire of viral genotypes and dual infection with different viral genotypes. J Infect Dis. 1999 November; 180(5):1466-76 and Erratum in: J Infect Dis 1999 November; 180(5):1756. Expression of the Epstein-Barr virus encoded EBNA-1 gene in stably transfected human and murine cell lines. Int J Cancer 42:592-8). This indicates that exact sequence fidelity is not required for their function. Despite sequence divergence, there is a retained functional correspondence between EBV and KSHV viruses (Moore, P. S., and Y. Chang. 2001. Kaposi's sarcoma-associated herpesvirus, p. 2803-2833. In D. Knipe, P. Howley, D. Griffin, R. Lamb, M. Martin, and S. Straus (ed.), Fields Virology, Fourth ed, vol. 2. Lippincott, Williams & Wilkins, Philadelphia) and thus immune escape mechanisms employed by EBV may apply to KSHV. Unlike EBNA1, which has been exhaustively studied as a CTL target, no investigations into CTL responses to LANA1 have been reported.

Since an effective KSHV vaccine should target viral latency to eliminate infection, enhanced Cell-Mediated Immunity (CMI) sensitization against latent antigens is an important vaccine strategy. Natural KSHV infection is capable of generating a CMI that is sufficient to prevent clinical disease—but not eliminate infection—in healthy adults, since most KSHV-infected adults are asymptomatic. Despite KSHV's armamentarium of immunoevasion proteins, provoking heightened CMI against KSHV latent proteins is thought to be sufficient to prevent tumor development in immunosuppressed patients or to even kill latently infected reservoir cells in healthy adults.

### Example 1

### Protein Processing of LANA1

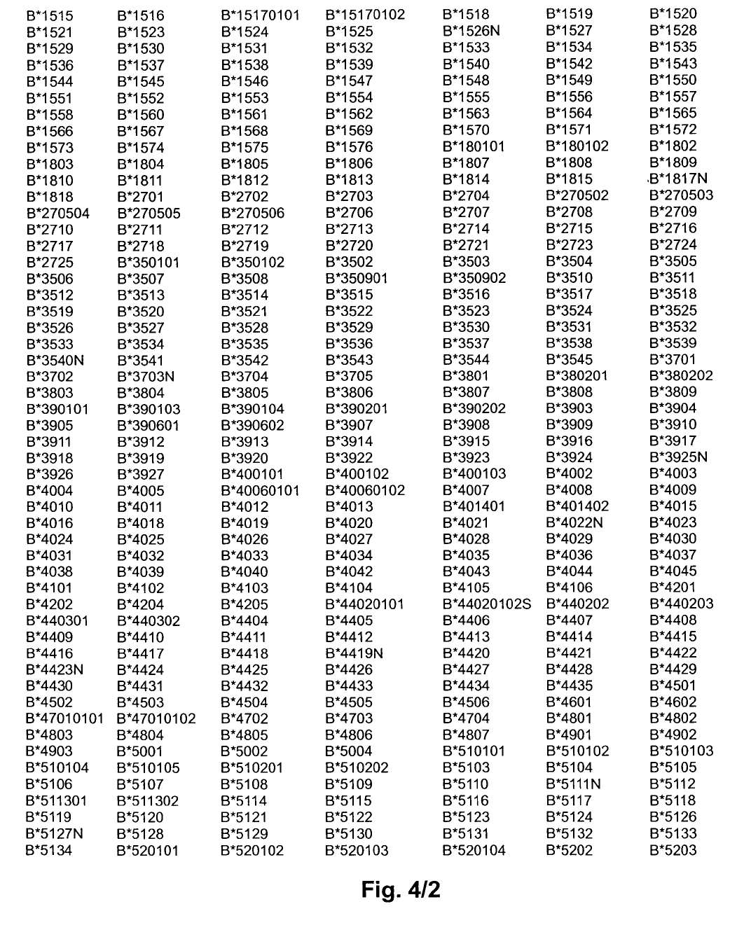

FIG. 5A shows immunoblotting for the ˜222-234 kD LANA1 protein. KS patient sera (left) and seronegative blood donor sera (right) using BC-1 cells and a panel of EBV⁺/KSHV-lymphoid cell lines. Note the reactivity of EBNA1 bands (Prior art, from Gao, S.-J., L. Kingsley, D. R. Hoover, T. J. Spira, C. R. Rinaldo, A. Saah, J. Phair, R. Detels, P. Parry, Y. Chang, and P. S. Moore. 1996. Seroconversion to antibodies against Kaposi's sarcoma-associated herpesvirus-related latent nuclear antigens before the development of Kaposi's sarcoma. New Eng J Med 335:233-241). FIG. 5B shows Northern blotting for ORF73 mRNA in PEL cell lines. Minimal ORF73 encoding mRNA is constitutively present in PEL cells. The polycistronic latency locus encodes at least two transcripts: a high molecular weight LT1 (ORFs 73, 72 and K13) and a lower molecular weight L2T (ORFs 72 and K13 alone). Both LT1 and LT2 transcripts are constitutively present under latent and lytic conditions whereas only LT1 is detected with a LANA1/ORF73 probe (prior art, from Sarid, R., J. S. Wiezorek, P. S. Moore, and Y. Chang. 1999. Characterization and cell cycle regulation of the major Kaposi's sarcoma-associated herpesvirus (human herpesvirus 8) latent genes and their promoter. J Virol 73:1438-46).

While LANA1 protein is readily detected in PEL cells by immunoblotting (FIG. 5A), ORF73 message is generally found at low levels in PEL cells compared to other cellular or viral messages by northern blotting (FIG. 5B). The scant amount of latency transcript (LT1) mRNA encoding vFLIP, vCYC and LANA1 (Dittmer, D., M. Lagunoff, R. Renne, K. Staskus, A. Haase, and D. Ganem. 1998. A cluster of latently expressed genes in Kaposi's sarcoma-associated herpesvirus. J Virol 72:8309-15) is transcribed from the KSHV LT promoter in a cell cycle dependent fashion with maximal expression in late G1 (Sarid, R., J. S. Wiezorek, P. S. Moore, and Y. Chang. 1999. Characterization and cell cycle regulation of the major Kaposi's sarcoma-associated herpesvirus (human herpesvirus 8) latent genes and their promoter. J Virol 73:1438-46). Intriguingly, EBV EBNA1 Qp promoter transcription has also been subsequently shown to be cell-cycle dependent (Davenport, M. G., and J. S. Pagano. 1999. Expression of EBNA-I mRNA is regulated by cell cycle during Epstein-Barr virus type I latency. J Virol 73:3154-61). Despite cell-cycle variation in mRNA abundance, these viral episome maintenance proteins have stable expression. For EBNA1, this is due to the prolonged stability of the EBNA1 protein which has been estimated to have a half-life of >36 hours (Davenport, M. G., et al. 1999. J Virol 73:3154-61). Only small amounts of EBNA1 mRNA are required to maintain steady-state levels of EBNA1 protein because of the protein's unusual stability.

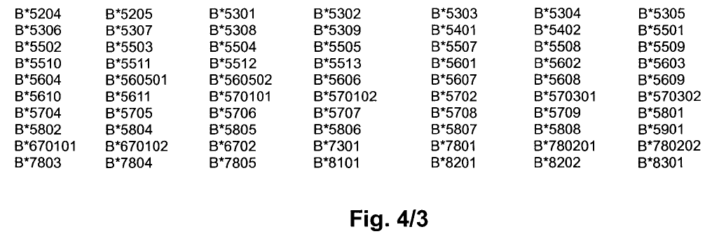

To determine if LANA1 protein has similar stability, we first examined naturally-infected PEL cell lines (BC-1, BCBL-1 and BCP-1) using a commercial rat monoclonal antibody (ABI) raised against the C-terminal fragment and our mouse monoclonal antibody (FIG. 6). LANA1 was immunoprecipitated with mouse mAb and detected with rat mAb. Note the 150-180 kDa bands are present detected by both antibodies and, like the ˜222-234 kDa bands, show polymorphic variation between cell lines. LANA1 migrates at ˜222-234 kDa but also has 4-5 distinct shoulder bands migrating between 150-180 kDa. These immunoblot patterns are interpreted as aberrant migration since the predicted size of LANA1 is only 135 kDa and that the shoulder bands are LANA1 degradation products. As seen in FIG. 6, immunoprecipitation with the mouse mAb and immunoblotting with the rat mAb against LANA1 detects both full-length protein and 150-180 kDa shoulder bands. LANA1 polymorphisms in different viral clones result from loss or gain of repeat units from the central repeat region as shown by protein immunoblotting and DNA sequence analysis (Gao, S. J., Y. J. Zhang, J. H. Deng, C. S. Rabkin, O. Flore, and H. B. Jenson. 1999. Molecular polymorphism of Kaposi's sarcoma-associated herpesvirus (Human herpesvirus 8) latent nuclear antigen: evidence for a large repertoire of viral genotypes and dual infection with different viral genotypes [published erratum appears in J Infect Dis 1999 November; 180(5):1756]. J Infect Dis 180:1466-76 and Zhang, Y. J., J. H. Deng, C. Rabkin, and S. J. Gao. 2000. Hot-spot variations of Kaposi's sarcoma-associated herpesvirus latent nuclear antigen and application in genotyping by PCR-RFLP. J Gen Virol 81:2049-2058) and size variation for the 150-180 kDa shoulder bands mirrors variation in the full-length LANA1 protein (see FIG. 6). The consistent spacing and immunoprecipitation of the 150-180 kDa bands with two different anti-LANA1 mAbs suggests that either LANA1 degradation generates precise 150-180 kDa subfragments (i.e. there is a proteolysis-resistant peptide core) or that the 150-180 kDa bands are actually stable isoforms of LANA1.

### Example 2

### LANA1 Turnover Studies

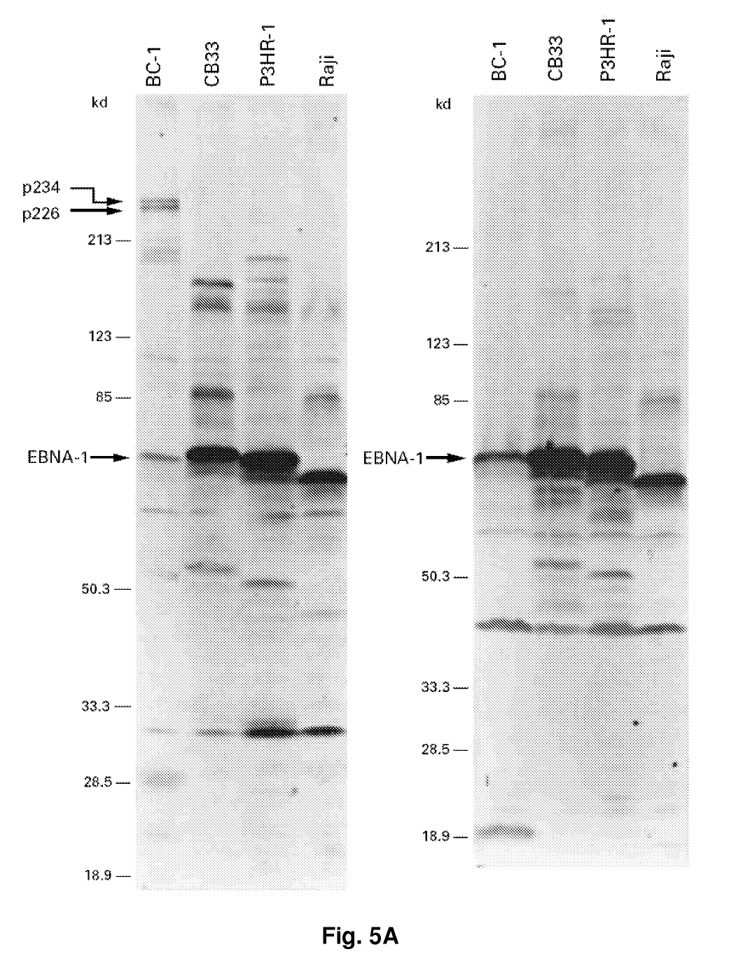

LANA1 turnover was next examined in PEL cells using two different methods. PEL cells were first treated with cycloheximide to abolish new protein synthesis and immunoblotted over a time course. FIGS. 7A-7D show a time course for LANA1 turnover after cycloheximide treatment in BCBL1 cells. FIG. 7A shows a LANA1 immunoblot of DMSO treated mock control. FIG. 7B shows a LANA1 immunoblot at different time points after 50 μg/ml cycloheximide treatment. Note loss of ˜220/230 kDa bands after 12 hours but detectable 150-180 kDa LANA1 bands up to 48 hours. In FIGS. 7C and 7D, respectively, the blots of FIGS. 7A and 7B stripped and re-probed with anti-IRF3 (cellular protein). As can be seen in FIGS. 7A-7D, the t_{1/2} for LANA1 is ˜12-24 hours compared to 3-6 hours for the cellular IRF3 protein. Importantly, there is a clear shift from the ˜220-234 kDa bands to the 150-180 kDa bands at later time points (24-48 hours).

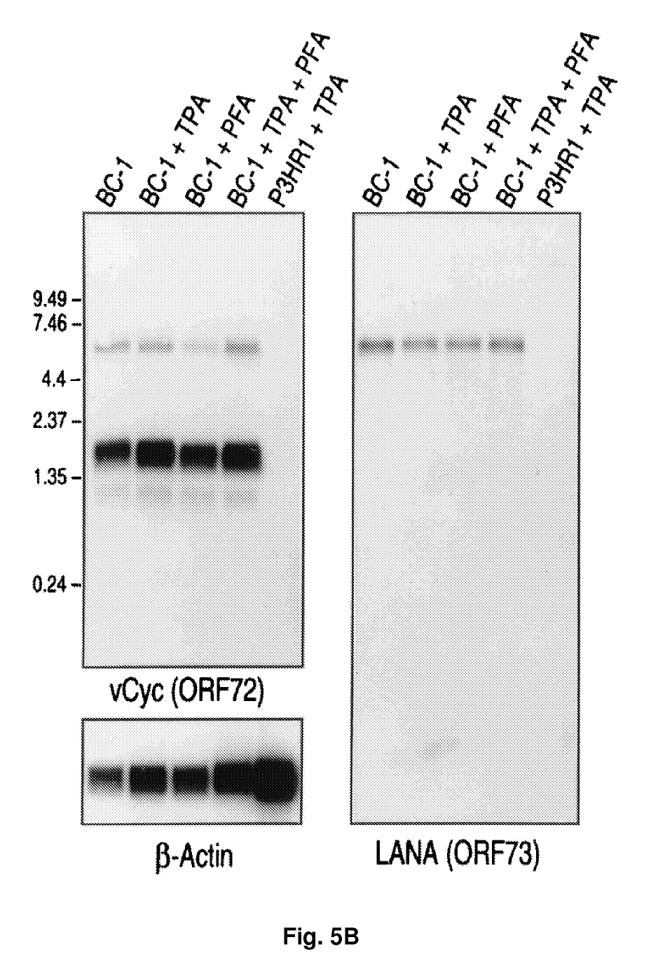

Nearly identical results are seen after treatment of BCP-1 cells (not shown). Since cycloheximide disrupts cellular metabolism causing cell death, we sought to verify these results using [³⁵S]-methionine pulse-chase labeling followed by LANA1 immunoprecipitation (FIG. 8). Pulse-chase experiments again show a prolonged half-life for LANA1 protein (t_{1/2}˜24 hrs) but there is no apparent shift from the high to the low molecular weight bands as was seen with cycloheximide treatment. These data show that LANA1, like EBNA1, has an extraordinarily long half-life in virus-infected cells.

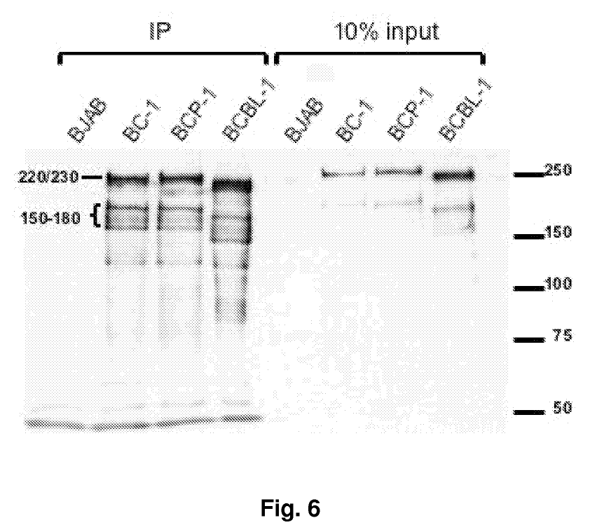

Since the half-life of LANA1 is prolonged, we next sought to determine if LANA1 also possess degradation-inhibition domains analogous to the EBNA1 GAr repeat sequences. To identify these domains, LANA1 fragments containing FLAG-epitopes were expressed in 293 cells in the presence and absence of a proteasome inhibitor (MG132). Turnover was then measured by immunoblotting over a time course. FIGS. 9A and 9B show that the LANA1 central repeat region inhibits LANA1 proteasomal degradation. FIG. 9A shows cycloheximide-treatment 293 cells expressing LANA1 and ORF45 protein. ORF45 protein shows rapid degradation (t_{1/2}˜1 hr) whereas LANA1 has minimal change over a 4-hour time course. FIG. 9B shows that MG132 treatment (+) has no affect on full-length LANA1 (top) but markedly increases steady-state protein levels for the LANA-NC fusion protein, the N-terminus fragment, and the C-terminus fragment.

As in naturally-infected PEL cells, when LANA1 is expressed in 293 cells, it has a prolonged half-life compared to KSHV ORF45 protein (FIG. 9A). Full-length LANA1 shows minimal accumulation in the presence of the proteasome-inhibitor MG132 whereas both N-terminal (1-312 aa) and C-terminal (938-1162 aa) fragments as well as an N—C fusion protein lacking the central repeat region have dramatically increased accumulation in the presence of proteasome inhibitors (FIG. 9B), indicating that the central repeat region is responsible for the reduced turnover of LANA1 protein.

These data are the first direct indication that LANA1 has aberrant protein stability. Also like EBNA1, degradation-inhibition is localized to an internal repeat region of LANA1 although there is no amino acid similarity between the two proteins. Both viral proteins are obligately expressed in latently-infected cells and appear to limit viral antigen proteolytic processing to blunt an effective CD8⁺ immune response.

### Example 3

### DI-Domain Stabilization of Destabilized GFP

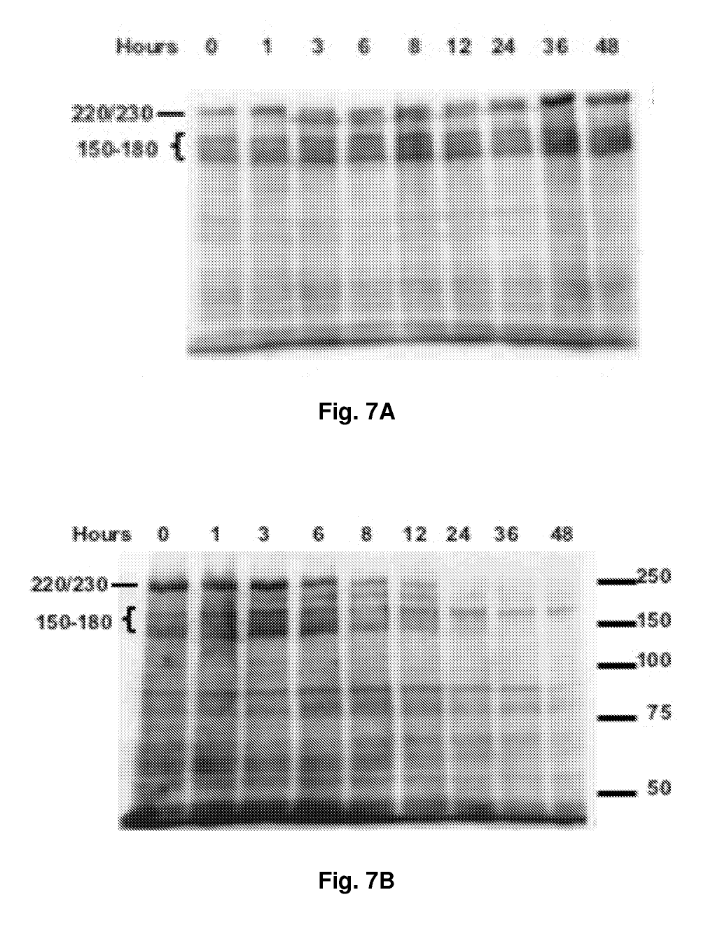

Based on this data suggesting that the degradation-inhibition (“DI”) domain is in the central repeat region, we next examined a fusion protein construct in which the CR2/CR3 region was cloned as a fusion partner to a destabilized GFP protein (dsGFP) (FIG. 10, where fusion protein is labeled as “CR2-CR3/N-dsGFP”). This central repeat fragment was chosen based on our parallel investigations into synthesis retardation functions of the central repeat region (see below) and additional constructs are being cloned, as described below, and tested to fine-map both degradation-inhibition and synthesis-inhibition functions. The dsGFP is engineered to undergo proteosomal degradation via ornithine decarboxylase PEST sequences (pd1EGFP-N1, Clontech). As shown in FIG. 10, LANA1 CR2/CR3 stabilizes heterologous proteins and prevents PEST-mediated proteosomal degradation. FIG. 10 shows the kinetics of protein turnover for a de-stabilized GFP (FIG. 10, top) and a heterologous fusion protein with CR2/CR3 after cycloheximide treatment (FIG. 10, bottom). Fusion with CR2/CR3 markedly extends the half-life of dsGFP.

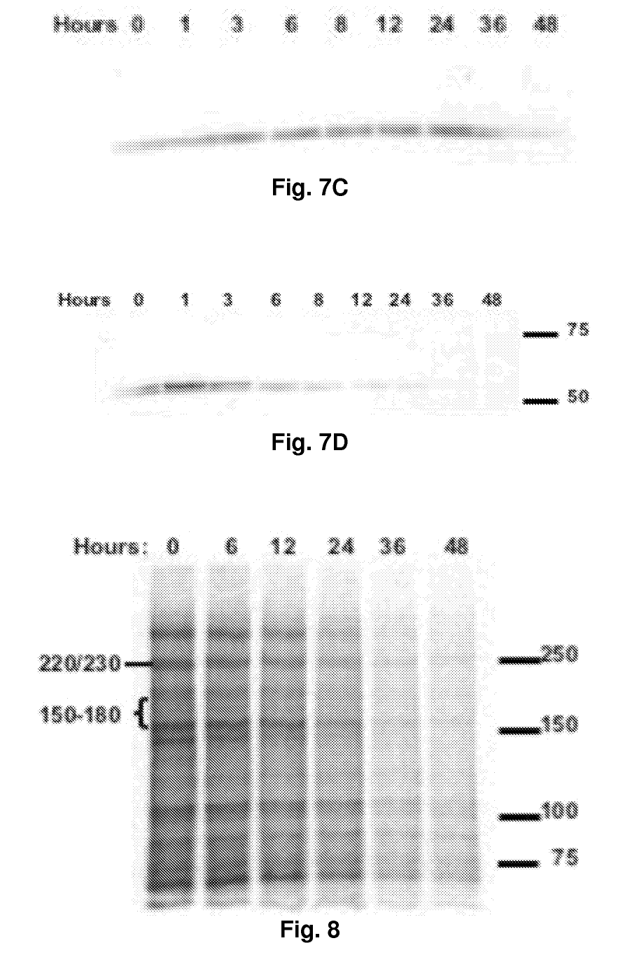

The parental dsGFP construct, as expected, has a short half-life of 1-2 hours after cycloheximide treatment to inhibit de novo protein synthesis (FIG. 10). In contrast, when CR2/CR3 is cloned in frame N-terminal to the dsGFP, the half-life of the peptide is approximately doubled and protein is readily detectable after 6 hours of treatment. Notably, mock DMSO-treated dsGFP shows marked protein accumulation over time but the CR2/CR3-GFP fusion protein does not. This is reminiscent of early studies on EBNA1 to determine if the GAr domain has synthesis-inhibition functions as well as degradation-inhibition functions. The reduced turnover of the CR2/CR3 fusion protein is readily seen by fluorescence microscopy (FIG. 11, right panels) in which virtually all parental dsGFP has been degraded in cells treated for 12 hours with cycloheximide while robust expression is still present for the CR2/CR3-GFP fusion protein. FIG. 11 illustrates CR2/CR3 inhibition of dsGFP turnover. Cells transfected with constructs described in FIG. 10 were examined for GFP fluorescence at time 0 and at 12 hours after CHX treatment. Note that both cells have similar levels of GFP expression at time 0. By 12 hours, dsGFP is nearly absent (top panels) but the CR2/CR3 fusion protein (bottom panels) is still readily detectable. The merged phase contrast images show cell numbers.

### Example 4

### Potential for Anti-LANA CD8⁺ Immune Responses

If LANA1 were forced to undergo proteolytic processing, it is likely to encode multiple antigenic peptides that could elicit CD8⁺ immune responses. Using the NetChop 2.0 (www.cbs.dtu.dk/services/NetChop/) and RANKPEP programs (http://immunax.dfci.harvard.edu/Tools/), we examined predicted proteolysis and HLA-restricted peptide presentation for the common HLA allele, HLA-A2 present in about 50% of Caucasians in US population. Thirty randomized hypothetical proteins having identical amino acid composition as LANA1 were generated using the Swiss ExPaSy proteomics server (http://www.expasy.org/) for comparison. Surprisingly, LANA1 has a significantly a greater number of predicted antigenic cleavage products (289) than any of the randomized proteins (avg=253, range 243-269, p<0.05). For example, when examined for predicted HLA-A2 (A*0201), 16 total likely HLA-A*0201 epitopes were identified including a repeat peptide from the leucine zipper domain represented multiple times. These results suggest that if LANA1 is destabilized, it is likely that enhanced protein turnover will elicit an effective CD8⁺ CTL immune response.

### Example 5

### LANA1 Synthesis Retardation

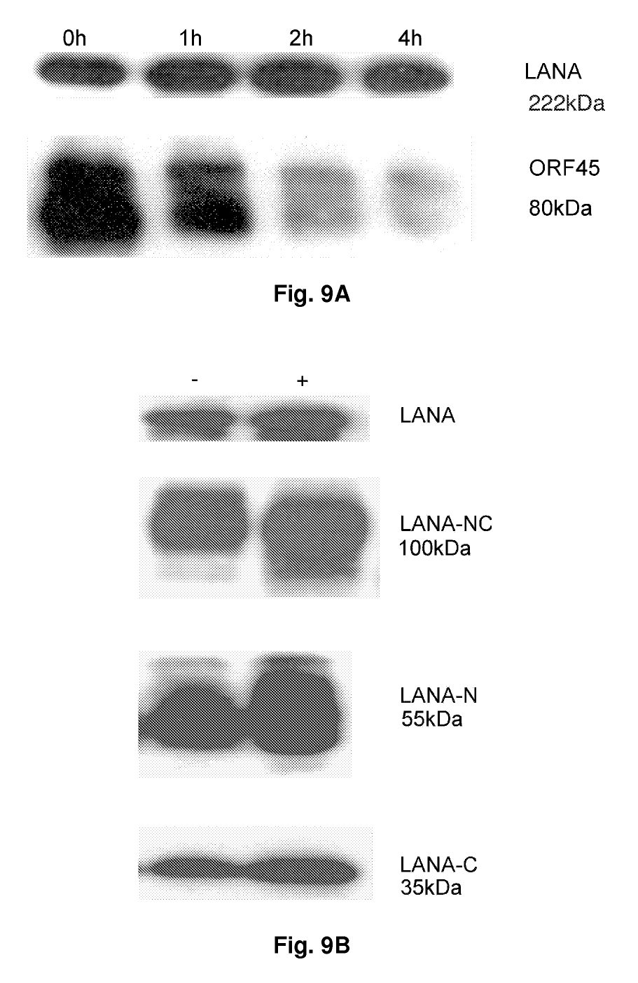

In addition to degradation-inhibition, EBNA1 GAr retards its own synthesis in cis to limit EBNA1 DRiP processing. We sought to determine if LANA1 also has a similar synthesis retardation (SR) domain. Unlike standard in vitro translation experiments, we performed uncoupled transcription and translation in which the molar amounts of T7 polymerase synthesized RNA were first determined so that equal amounts of transcripts are added to each translation reaction. To minimize variability, C-terminal deletion constructs were derived from an identical LANA1 full-length parental construct using unique or infrequent restriction enzyme sites within LANA1. As shown in FIGS. 12A and 12B, in vitro translation is markedly inhibited in constructs containing the DEQ-rich CR2 domain suggesting this region behaves in an analogous fashion to the EBNA1 GAr. FIG. 12A shows in vitro translation mapping of LANA1 synthesis retardation domains. C-terminal deletion constructs (FIG. 12B) up to the DEQ-rich CR2 domain are efficiently translated (constructs 1-320/HincII and 1-43/Ac1I). The 1-464/SbfI fragment containing 4 repeats from the CR2 region is synthesized at only 40% of the Ac1I fragment as measured by densitometry. Further retardation occurs in longer 1-928/BsmBI, 1-980/NruI and full length (1-1162) constructs. RNAs for all fragments were pre-measured to ensure equal abundance of transcripts for each translation reaction. Translation reactions were performed using equimolar amounts of T7 pol transcribed mRNA and electrophoresed through an 8% SDS polyacrylamide gel. Positive control luciferase RNA translates into ˜61 kDa product. Negative control is without RNA in in vitro uncoupled translation reaction. Importantly, the aspartate-glutamate rich CR1 region, which is highly similar to the aspartate-glutamate-glutamine rich CR2, does not inhibit translation since the 1-434 aa construct containing CR1 is efficiently translated.

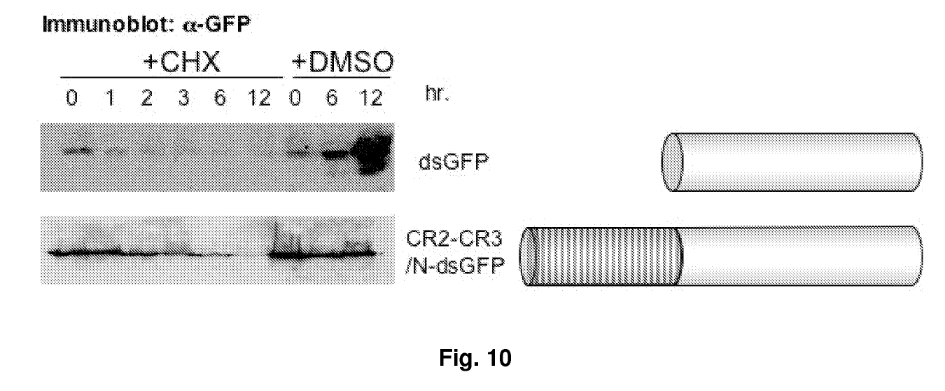

Additional evidence that specific domains of LANA1 retard protein synthesis is shown in FIG. 13. FIG. 13 shows that the LANA1 synthesis retardation domain is localized to the central repeat region. Specifically, the N- and C-terminal fusion protein lacking the central repeat region (312-938 aa) is efficiently synthesized, whereas full-length LANA1 protein shows clear evidence of synthesis inhibition. A LANA1 fusion protein, in which the N- and C-termini are joined, deleting the central repeat domain, is efficiently translated compared to the full-length LANA1 protein.

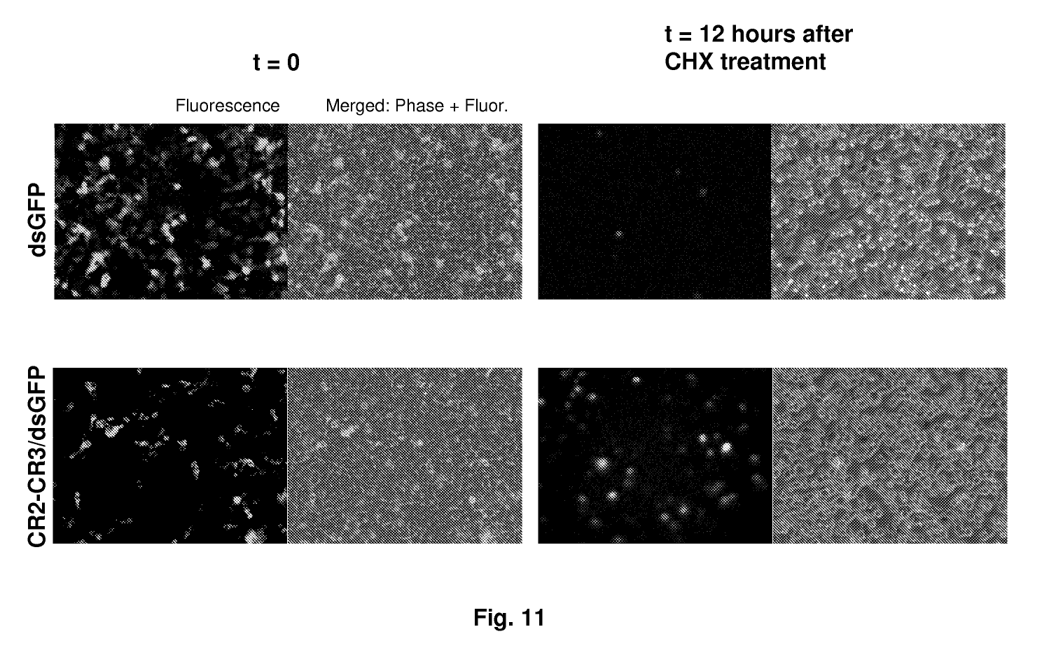

To characterize the SR domain, we examined in vitro synthesis of the CR2/CR3-GFP fusion constructs. Previous reports suggest that GAr must be fused N-terminal to a heterologous fusion partner to retard its translation (Yin, Y., B. Manoury, and R. Fahraeus. 2003. Self-inhibition of synthesis and antigen presentation by Epstein-Barr virus-encoded EBNA1. Science 301:1371-4), but we find that LANA1 CR2/CR3 retards translation regardless of whether it is fused 5′ or 3′ of a heterologous gene sequence (FIG. 14). FIG. 14 illustrates that CR2/CR3 retards translation of EGFP in both the N-terminal and the C-terminal positions. This in vitro translation compares dsEGFP fusion proteins with either N-terminal LANA or with CR2C3. Both orientations of the 80 kD N-terminal LANA fusion protein, but not the CR2/CR3 fusion protein (arrow at expected size), are expressed. In this experiment we have compared synthesis of the CR2/CR3-GFP fusion protein to the N-terminal LANA1-GFP fusion protein in both orientations. This data confirms our previous observation that the synthesis-retardation domain is localized to the repeat region but also shows that synthesis retardation occurs regardless of CR2/CR3-GFP orientation. We are in the process of trying to confirm the previously reported orientation-dependent results for GAr by cloning the EBNA1 fragment into the same vector construct to allow direct comparison to CR2/CR3. If EBNA1 and LANA1 behave differently in their synthesis retardation characteristics, this may indicate that these two viral proteins perform self-inhibition of synthesis through different mechanisms.

### Example 6

### Mechanism of Synthesis Retardation

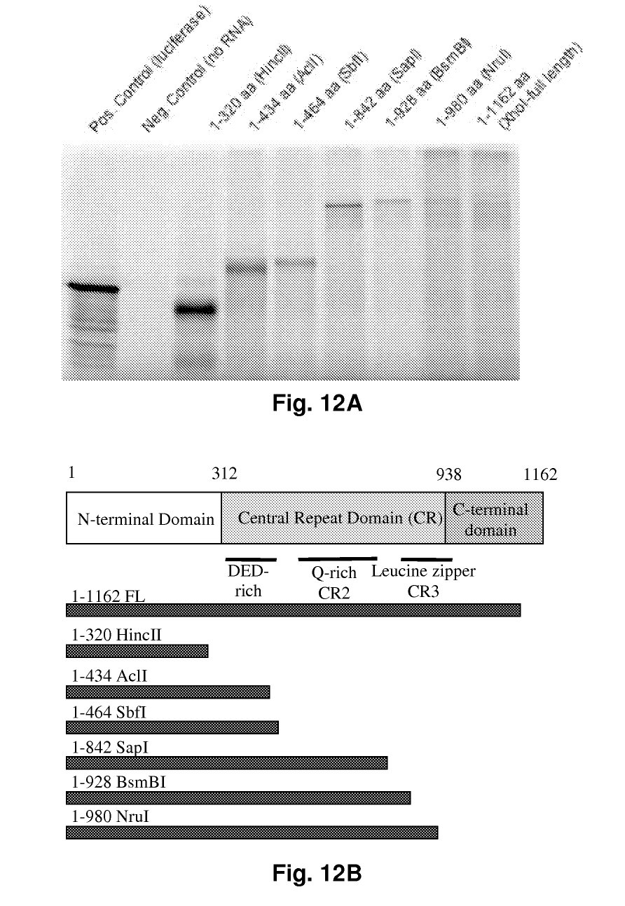

The mechanism for translational autoinhibition of viral protein synthesis is unknown. One possibility is that the highly repeated DEQ motif may locally deplete D, E and Q amino acids, requiring cytosolic diffusion of loaded cognate D, E, and Q amino-acyl-tRNAs to the ribosomal machinery for continued elongation of the peptide chain. Charged amino acids are loaded onto cognate tRNAs through specific ATP-dependent aminoacyl-tRNA synthetases during in vitro translation in reticulocyte lysates. If local concentrations of amino acids are rate-limiting, then amino acid supplementation should increase full length protein synthesis. FIGS. 15A and 15B show the effect of amino acid supplementation on LANA1 fragment synthesis. In FIG. 15A, a standard amino acid reaction mixture (Promega) is used. In FIG. 15B, excess D, E and Q in excess (1 mM) are provided. In vitro translation was performed on fragments shown in FIG. 10 and [³⁵S]-methionine incorporation quantitated using a PhosphorImager. Relative LANA1 protein fragment synthesis is similar when either no amino acid supplementation is used or when DEQ amino acids are added in excess suggesting amino acid concentrations are not rate limiting for LANA1 synthesis.

Another possibility is that rare codons usage by viral proteins might retard, but not inhibit, viral protein synthesis. Codon usage bias toward rare human codons has been previously reported for tumor viruses although an obvious benefit for replication fitness has not been described (Zhao, K. N., W. J. Liu, and I. H. Frazer. 2003. Codon usage bias and A⁺T content variation in human papillomavirus genomes. Virus Res 98:95-104). This might be a previously unrecognized mechanism to decrease DRiP surveillance for constitutively-expressed viral proteins. Evidence to support this comes from studies of papillomavirus E7 DNA vaccines in which codon optimization increased CTL responses and antitumor activity (Liu, W. J., F. Gao, K. N. Zhao, W. Zhao, G. J. Fernando, R. Thomas, and I. H. Frazer. 2002. Codon modified human papillomavirus type 16 E7 DNA vaccine enhances cytotoxic T-lymphocyte induction and anti-tumour activity. Virology 301:43-52).

To examine this possibility, codon usage analysis for LANA1 was performed (www.GUAC.de) on BC-1 ORF73 sequence using the human gene table: uncommon codon usage is frequent throughout LANA1 with 32 rare (<10%) and 381 uncommon (<20%) codons. Overall, nearly one-fourth of all LANA1 codons are rare or uncommon potentially diminishing rates of LANA1 synthesis. Even if codon usage is not the reason per se for the CR2 retardation of LANA1 synthesis, codon optimization might partially over come this inhibition and increase LANA1 DRiP formation.

These results show that LANA1 protein processing is similar to EBNA1 although the two proteins share no amino acid similarity. LANA1, like EBNA1, possesses structural features that allow the protein to escape immune processing. Both LANA1 and EBNA1 possess central repeat domains that inhibit proteasomal processing of the full-length proteins. Additionally, central repeat regions retard protein synthesis. We have localized this domain in LANA1 to a portion of the CR2 domain. If the results regarding EBNA can be extrapolated to LANA1, then the LANA1 CR2 region also likely is responsible for degradation inhibition. While EBNA1 primary structure, particularly the placement of alanine residues, has been shown to be important for these effects, our data suggests that unrelated peptide sequences can play a similar role. Indeed, while the G and A amino acid residues of the EBNA1 repeat domain have small, non-polar side chains, the D, E and Q residues of LANA1 CR2 are strongly polar, with terminal carboxyl or amide groups.

### Example 7

### Analysis of Structural Features that Inhibit LANA1 Proteosomal Processing

The data and examples above suggest that the primary sequence of LANA1 reduces its turnover and rate of synthesis, diminishing the likelihood for cell-mediated immune recognition. Identifying the responsible domains and engineering LANA1 antigens that have enhanced proteolytic processing may be critical for development of an effective KSHV vaccine. These experiments, together with data from other viral proteins, will also provide insights in basic aspects of protein processing in immune recognition.

In one experiment, we can analyze structural features that inhibit LANA1 proteasomal processing. The EBNA1 Gly-Ala repeat (GAr) is the only viral protein sequence thoroughly investigated for its proteasomal inhibition properties, although similar functional domains have been described for long-lived cellular proteins (Heessen, S., M. G. Masucci, and N. P. Dantuma. 2005. The UBA2 domain functions as an intrinsic stabilization signal that protects Rad23 from proteasomal degradation. Mol Cell 18:225-35). We have shown that LANA1 shares many features with EBNA1 but it does not have a GAr sequence required for this function (Sharipo, A., M. Imreh, A. Leonchiks, C. Branden, and M. G. Masucci. 2001. cis-Inhibition of proteasomal degradation by viral repeats: impact of length and amino acid composition. FEBS Lett 499:137-42). Fine mapping studies show that the EBNA1 GAr proteasome inhibition is proportional to the number of Gly-Ala repeats and the position of repeated alanine residues may be critical for this effect (Sharipo, A., M. Imreh, A. Leonchiks, C. Branden, and M. G. Masucci. 2001. cis-Inhibition of proteasomal degradation by viral repeats: impact of length and amino acid composition. FEBS Lett 499:137-42). Defining a LANA1 degradation-inhibition (DI) domain provides a second example that can be compared to EBV GAr in understanding the mechanisms of viral protein stability. Although proteins from different viruses may have analogous functions, their mechanisms of action or targets may be very different. Methods are described below for defining the mechanism and sequences responsible for LANA1 stability that may or may not be mechanistically similar to EBNA1. This line of investigation may lead to important insights into similar structures in cellular proteins resulting in prolonged post-translational stability.

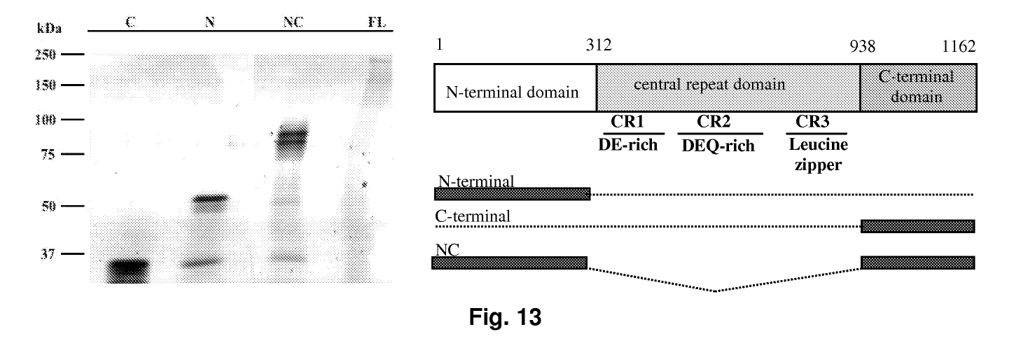

The examples above and FIG. 16 show LANA1 deletion and heterologous fusion constructs used in certain of the Examples herein. As indicated above, portions of the central repeat region appear to be involved in degradation-inhibition. Each construct is either a terminal deletion or a fusion of N- and C-termini with an intervening region deleted. Five LANA1 regions, cloned independently or in combination using an N-terminal FLAG tag vector, include:

the N-terminus (1-312 aa),

the DE-rich central repeat (CR) 1 (CR1, 313-428 aa),

the DEQ-rich CR2 (429-768 aa),

the leucine zipper containing CR3 (769-938 aa), and

the C-terminus (939-1162 aa).

Comparison also may be made to the EBNA1 GAr domain. Efforts are underway to clone EBNA1 GAr into the vector backbones for which LANA1 domains have been cloned. This may allow for a more direct comparison of motifs of these two viral proteins. Simultaneous comparisons can be made to endogenous β-actin turnover (half-life of 9 hours (Brinster, R. L., S. Brunner, X. Joseph, and I. L. Levey. 1979. Protein degradation in the mouse blastocyst. J Biol Chem 254:1927-31)) to monitor global effects on protein turnover.

Turnover of each deletion construct may be compared to full-length LANA1 protein using [³⁵S]-methionine pulse-chase labeling followed by immunoprecipitation. Control proteins include full-length LANA1, cellular IRF3, or other appropriate cellular proteins. Alternatively, cycloheximide time courses followed by immunoblotting can be performed as described above. The LANA1 mouse mAb described above is an excellent immunoblotting and immunoprecipitating antibody. Used in combination with commercially available rat anti-LANA1 mAb (Kellam, P., D. Bourboulia, N. Dupin, C. Shotton, C. Fisher, S. Talbot, C. Boshoff, and R. A. Weiss. 1999. Characterization of monoclonal antibodies raised against the latent nuclear antigen of human herpesvirus 8. J Virol 73:5149-55; ABI), anti-FLAG (Sigma) and anti-EGFP (Zymed Laboratories Inc.) antibodies, we may be able to readily measure and contrast turnover kinetics for all deletion constructs. Deletion constructs have been generated with identical 3′ and 5′ UTRs to assure comparability. Once the DI peptide region(s) responsible for delayed turnover and/or synthesis is confirmed, fine deletions of this peptide region can be used to determine the required minimal motif for protein degradation inhibition. This can allow generation of a full-length LANA1 construct differing only in the minimal domain mediating protein longevity.

Once the optimal LANA1 ΔDI protein is identified (i.e., the smallest deletion construct having fastest degradation kinetics), then LANA1 immunoprecipitation and ubiquitin-blotting (Abcam AB7780) in the presence and absence of proteasome inhibitors (e.g. MG132) would provide information on whether the DI motif inhibits ubiquitinylation of LANA1 or whether ubiquitinylation occurs, but the DI prevents proteasomal processing (as is the case for the GAr).

In another experiment, the DI domain and N- and C-terminal fusions may be cloned into the 5′ UTR of an IκB-α expression construct. This approach has been used by Sharipo et al. for analysis of effects of EBNA1 GAr on ubiquitinylation. IκB-α undergoes rapid ubiquitin-mediated proteolysis after TNF-α stimulation and this assay will allow direct comparison of the LANA1 DI domain to the EBNA1 GAr (Sharipo, A., M. Imreh, A. Leonchiks, S. Imreh, and M. G. Masucci. 1998. A minimal glycine-alanine repeat prevents the interaction of ubiquitinated I kappaB alpha with the proteasome: a new mechanism for selective inhibition of proteolysis. Nat Med 4:939-44). These studies could demonstrate whether or not the DI domain is transferable to other proteins. As indicated above, immunoprecipitating IkB using a FLAG antibody in the presence of MG132 and performing ubiquitin immunoblotting could demonstrate whether or not the DI inhibits ubiquitinylation of heterologous proteins. Ubiquitinylation likely would appear as a ladder of higher molecular weight forms.

As an alternative approach, candidate DI domains are being cloned as fusion proteins into a destabilized GFP expression vector. As shown above, the putative DI domain in CR2/CR3 markedly inhibits degradation (see FIG. 10) and portions of the LANA CR will be systematically examined to fine-map DI activity. Assays for providing evidence for ubiquitinylation in the presence and absence of proteasome inhibitors can be performed as described above to determine whether the DI domain inhibits ubiquitinylation. Similarly, the DI domain can be cloned as N- and C-terminal fusions with chicken ovalbumin (OVA), and the kinetics of the fusion protein turnover can be tested in EL4 cells compared to native OVA by [³⁵S]-methionine pulse-chase and for in vitro CTL studies using chromium release assays.

Mechanistic studies: Follow-up studies to examine proteasome interaction would be dependent on results from the experiments outlined above. Either the DI inhibits the ubiquitin signal from being attached to the target protein or ubiquitinylation takes place, but the DI prevents normal proteosomal processing of the protein. In the former case, the DI may prevent E3 ligase-target protein interaction while in the second case, the DI may prevent target protein-proteasome interaction. Several possible experimental paths are described here; the actual analysis would be dependent on preliminary data determining whether or not the DI inhibits cis protein ubiquitinylation.

If the DI inhibits cis protein ubiquitinylation, then the DI may be cloned in fusion with a defined protein having a well-described E3 ligase to see if the DI sequence prevents E3 recognition. For example, p53 and its E3 ligase MDM2 and E4 ligase p300 (Grossman, S. R., M. E. Deato, C. Brignone, H. M. Chan, A. L. Kung, H. Tagami, Y. Nakatani, and D. M. Livingston. 2003. Polyubiquitination of p53 by a ubiquitin ligase activity of p300. Science 300:342-4) can be used. The DI domain may be cloned into p53, then p53 ubiquitinylation can be determined by immunoprecipitation and ubiquitin immunoblotting in the presence of proteasome inhibitors. If the DI domain inhibits p53 ubiquitinylation, then p53 immunoprecipitation and MDM2 or p300 blotting will be performed to determine whether or not the DI domain prevents p53-MDM2 or p53-p300 interaction. These experiments may potentially help to determine whether or not the DI inhibits target protein-E3 ligase interaction as a means to prevent protein degradation.

If LANA DI does not inhibit ubiquitinylation but does prevent proteasomal degradation, then the DI fusion protein and its parental partner may be FLAG immunoprecipitated in the presence of MG132 and immunoblotted with anti-20 S subunit antibodies (Clone 21D11, Invitrogen) to determine if the DI fusion protein interacts with the proteasome. If no interaction is seen, this will be similar to the EBNA1 GAr, which does not prevent ubiquitinylation but does prevent ubiquitinylated targets from interacting with the 20 S proteasome.

Cis vs. trans inhibition of degradation can be examined using expression constructs containing the DI domain (preliminary data show that CR2/CR3 encodes this domain). We could co-express a dsGFP and either LANA1 CR2/CR3 or the LANA1 N-terminus fragments on separate plasmids or together as fusion proteins. Turnover of GFP protein can be measured by a cycloheximide time course immunoblot. We anticipate if the DI acts in cis, there will be no difference in GFP turnover in the presence of LANA1 CR2/CR3 compared to LANA1 N-terminus, which lacks the DI domain. In contrast the fusion protein expressing CR2/CR3 in cis should have markedly delayed turnover as shown in FIG. 10. If, however, the DI domain inhibits turnover in trans, then the GFP could have reduced turnover when CR2/CR3 is expressed from a separate plasmid but not when N-terminus is expressed on a separate plasmid.

Alternatively, cycloheximide turnover studies can be performed by coexpression of LANA1 and LANA1 ΔDI. In this case, if DI occurs in trans, we anticipate that the LANA1 ΔDI will have reduced turnover when increasing amounts of full-length LANA I protein is coexpressed. If DI acts in cis, then there will be no effect when full-length LANA1 or luciferase (negative control) is expressed in trans. To address the possibility of trans saturation effects, these experiments might be performed using constant amounts of LANAΔDI and increasing amounts of full-length LANA1. To also control for trans-saturation effects, this can be performed by expression of increasing amounts of an irrelevant protein (luciferase) instead of full-length LANA1. As is a standard in our laboratory, all transfection have the same total amount of transfected DNA to avoid promoter competition. If degradation of LANAΔDI is inhibited by coexpression of LANA1, this will be evidence for trans inhibition of degradation.

We anticipate the data from heterologous DI fusion proteins and from LANA1 deletion proteins will show similar results, i.e. the DI either inhibits metabolism of other proteins (trans) or only when directly fused to a particular protein (cis). These experiments are technically straight-forward, complementary to each other and will yield unambiguous results.

### Example 8

### Analysis of LANA1 Synthesis Retardation (SR) Domain and LANA1 DRiP Formation

The data presented herein shows that the DEQ-rich CR2 domain retards LANA1 synthesis in uncoupled in vitro translation assays (see FIGS. 12A and 12B). The domain responsible for synthesis retardation is referred to as the SR domain. These data are again reminiscent of the EBNA1 GAr (Yin, Y., B. Manoury, and R. Fahraeus. 2003. Self-inhibition of synthesis and antigen presentation by Epstein-Barr virus-encoded EBNA1. Science 301:1371-4), and there is evidence that synthesis-retardation may play a more critical role in EBNA1 evasion of CD8⁺ T cell recognition than proteasome processing inhibition (Tellam, J., G. Connolly, K. J. Green, J. J. Miles, D. J. Moss, S. R. Burrows, and R. Khanna. 2004. Endogenous presentation of CD8⁺ T cell epitopes from Epstein-Barr virus-encoded nuclear antigen 1. J Exp Med 199:1421-31). There has been no examination of synthesis retardation for LANA1 and, similar to the effects of EBNA1 GAr on proteolysis, investigation of LANA1 synthesis retardation provides an important second model for investigating DRiP processing mechanisms.

To confirm and extend our initial observations, the rate of LANA synthesis initially can be determined through quantitative decoupled in vitro transcription and translation analyses. To perform this, RNAs can be generated using T7 polymerase in in vitro transcription reactions and standardized molar amounts of RNA are used in each analysis. Equimolar amounts of each construct are then added to rabbit reticulocyte lysates in the presence of [³⁵S]-methionine, with synthesis stopped at specific time points. The reaction products are then run on SDS-PAGE gels. From this we can determine the relative rate of peptide synthesis for each construct. To extend these analyses, we can measure DRiP formation in vivo for LANA1 and derivative proteins using established protocols as described in detail below.

Using this method, the relative synthesis rates for full length LANA1, LANA1ΔN, LANA1 ΔCR1, LANA1 ΔCR2, LANA1ΔCR3 and LANA1ΔC (as described above) can be measured, as well as the control green fluorescence protein (GFP) in vitro. To control for the effect of RNA size, relative synthesis rates may be plotted for each construct. It is expected that LANA1 constructs lacking SR activity will have increased rates of protein accumulation which can be determined over a 90 min time course. It is expected that this more detailed analysis may confirm that the DEQ-rich CR2 encodes the SR domain. For EBNA1, GAr possesses both DI and SR functions. It has not been shown experimentally that the same is true for LANA1.

Cis vs. Trans Synthesis Retardation: It is possible that LANA1 acts in trans to retard its own synthesis. Activated PKR, for example, inhibits ribosomal translation in trans by phosphorylating the elongation factor EIF-2α(Wu, S., K. U. Kumar, and R. J. Kaufman. 1998. Identification and requirement of three ribosome binding domains in dsRNA-dependent protein kinase (PKR). Biochemistry 37:13816-26). Our experience, and that of others, with LANA1 coexpression suggests that LANA1 does not globally retard RNA synthesis and thus it is unlikely to retard synthesis in trans. Further, EBNA1 GAr inhibits translation in cis (Yin, Y., B. Manoury, and R. Fahraeus. 2003. Self-inhibition of synthesis and antigen presentation by Epstein-Barr virus-encoded EBNA1. Science 301:1371-4) and it is most likely that LANA1 behaves similarly.

To confirm the SR domain found in the above experiments, the deleted regions of LANA1 can be cloned as N- and C-terminal fusion proteins into GFP, and rates of in vitro synthesis can be determined using an in-lab VersaFluor Fluorometer (BioRad). It is anticipated that GFP fusion proteins containing the SR domain will have slower relative rates of synthesis. By cloning the SR domain at both N and C terminals, the role, if any, of the position of the SR domain on translational kinetics can be determined. Together with other experiments described herein, this will localize and define the SR domain and provide useful reagents for immunologic studies. Excellent progress has been made in cloning the first of these fusion protein reagents.

Evidence is presented herein that the CR2/CR3 domain inhibits in vitro translation (using rabbit reticulocyte lysates) regardless of whether this element is in the N- or C-terminal positions (see FIG. 14). This can be confirmed in vivo in the context of the cellular environment since this differs from the behavior of EBNA1 GAr which causes synthesis retardation when present at the N-terminal, but not the C-terminal, portion of heterologous proteins. This will provide evidence either that the two viral protein use different mechanisms of synthesis inhibition or that both viral proteins behave similarly. This will further allow a comparative investigation of the mechanisms for synthesis retardation that at this time are unknown. It also is anticipated that if the SR domain is confirmed to be the DEQ region, then the dose of repeats will determine the strength of the inhibition. This is an alternative approach to direct mutagenesis studies involving LANA1.

### Example 9

### In vivo Analyses for LANA1 DRiP Formation

DRiPs are immediately cleaved after synthesis by proteasomal degradation into oligopeptides which are then either surveyed by the MHC machinery or degraded into free amino acids by the actions of endo- and aminopeptidases (Qian, S. B., J. R. Bennink, and J. W. Yewdell. 2005. Quantitating defective ribosome products. Methods Mol Biol 301:271-81). While EBNA1 GAr synthesis retardation is assumed to decrease EBNA1 DRiPs, this has actually not been directly evaluated. DRiP formation is quantified by determining the ribosome-associated peptide fraction in the presence and absence of proteasome inhibitors. This is usually performed as a kinetic experiment; and peptides that do not form DRiPs show minimal change in the presence of proteasome inhibitors whereas unstable peptides undergoing DRiP processing show large changes in synthesis kinetics on addition of proteasome inhibitors (Qian, S. B., J. R. Bennink, and J. W. Yewdell. 2005. Quantitating defective ribosome products. Methods Mol Biol 301:271-81). We can measure this process by quantitating the ribosome-associated fraction of LANA1 in the presence and absence of proteasome inhibitors. If LANA1 is resistant to misfolding degradation, then proteasome inhibitors will minimally affect the ribosome associated LANA1, whereas LANAΔSRD will be markedly increased in the presence of proteasome inhibitors.

The DRiP protein compartment can be biochemically fractionated through ultracentrifugation (Schubert, U., L. C. Anton, J. Gibbs, C. C. Norbury, J. W. Yewdell, and J. R. Bennink. 2000. Rapid degradation of a large fraction of newly synthesized proteins by proteasomes. Nature 404:770-4). DRiPs are identifiable as proteasome-inhibition sensitive proteins associated with the ribosome fraction. To quantitate full-length LANA1 and LANAΔSRD DRiPs, constructs for LANA1 and LANAΔSRD can be transfected into 293 cells using Lipofectamine 2000 (Invitrogen). 48 hours post transfection, cells can be placed in pulse medium (methionine-free DMEM with a final concentration of 2.5 mCi of [³⁵S]-methionine) with and without 10 mM MG132. Following labeling for 30 minutes, cells can be washed twice with 10 volumes of ice-cold PBS containing 1 mg/ml cold L-methionine. Cells can then be resuspended in growth medium with and without 10 mM MG132. An initial t-0 sample can be withdrawn and the remaining cells can be incubated in a 37° C. water bath with shaking every 30 min. A final sample can be collected at 3.5 hours. To obtain ER-enriched fractions, cell pellets can be washed with PBS and resuspended in ice-cold TritonX-100 extraction buffer (50 mM Tris-Cl pH7.4, 150 mM NaCl, 1 mM EDTA and 1% TritonX-100). Following a 30 minute incubation on ice, samples can be centrifuged at 150,000 g×2 hours. Supernatants can be transferred to fresh tubes, and buffer without TritonX-100 can be added to the samples to bring down the concentration of TritonX-100 to 0.5%. These samples can then be subjected to immunoprecipitation using protein A sepharose beads with anti-GFP or anti-LANA1 antibody depending on the constructs. Following immunoprecipitation, beads can be washed with DRiP wash buffer (50 mM Tris-Cl 7.4, 150 mM NaCl, 1 mM EDTA and 0.5% TritonX-100) and the final eluate can be subjected to SDS-PAGE for LANA1 and GFP constructs respectively. Gels can be dried and exposed to phosphorimaging.

This standard protocol allows measurement of [³⁵S]-methionine labeled LANA1 in the DRiP compartment that is sensitive to proteosome inhibitors (Qian, S. B., J. R. Bennink, and J. W. Yewdell. 2005. Quantitating defective ribosome products. Methods Mol Biol 301:271-81). Standard protocols use scintillation counting to measure the recovered metabolically-labelled DRiPs.

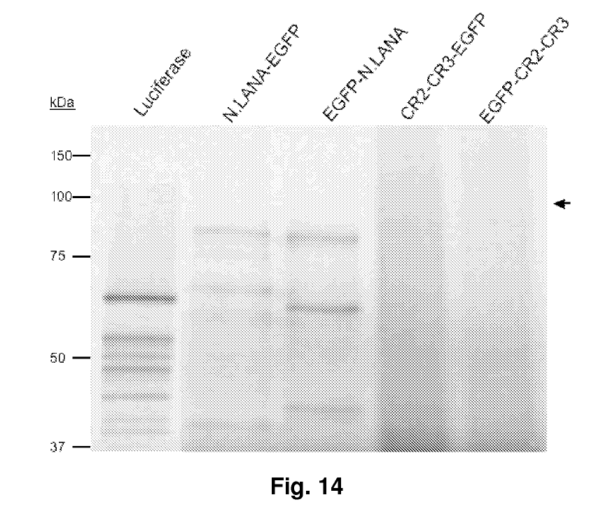

A hypothetical example of DRiP quantitation for LANA1 and LANAΔSRD is shown in FIG. 17 in which the kinetics of LANA1 and LANAΔSRD degradation are shown. If wild-type LANA1 has reduced DRiP formation, then treatment with proteasome inhibitors (MG132) will have minimal impact on the kinetics of LANA1 degradation. We hypothesize that LANAΔSRD will be susceptible to DRiP degradation and therefore there will be a larger impact of MG132 treatment on LANAΔSRD degradation kinetics. Typically, marked differences in DRiP degradation kinetics are seen 4-6 hours after MG132 treatment. As controls, we can compare LANA1 DRiP kinetics to EBNA1 and EBNAΔGAr, and to ovalbumin. This data will directly show 1) whether or not LANA1 is resistant to DRiP processing, and 2) whether or not the SRD domain that retards translation inhibits DRiP processing.

An alternative approach involves direct detection of GFP fusion protein DRiPs without antibody immunoprecipitation. As seen in FIG. 11, we readily detected differences in GFP protein stability when fused to CR2/CR3 by fluorescence microscopy. To measure the effect of LANA1 CR2/CR3 on DRIP formation, cells could be transfected with GFP-LANA1.N or GFP-LANA1.CR2/CR3 fusion proteins, treated with MG132 or mock, and the DRiP compartment protein isolated as described in the protocol above (a typical GFP construct rather than the destabilized version described in FIG. 11 can be used to reduce the effects of destabilized protein turnover). Rather than immunoprecipitating LANA1 protein, the SRD-containing or a negative-control domain fusion protein then can be directly quantitated by fluorescence after standardizing for ER protein content. In this case, we anticipate that fusion proteins containing the SRD will have minimal change in fluorescence after MG132 treatment while MG132 treatment will markedly increase the control peptide fusion protein. Standard controls, such as mock transfection and use of a dilution calibration curve would allow us to control for autofluorescence and to measure relative amounts of protein by this technique. It is expected that the SRD in full-length LANA1 will decrease the fraction of LANA1 that is proteasomally-processed and hence sensitive to proteasome inhibitors. This will result in minimal change in the degradation kinetics with proteasome inhibitors, whereas the LANAΔSRD lacking the SRD will show a marked increased accumulation in the presence of proteasome inhibitors. If LANA1 accumulation is highly sensitive to proteasome inhibitors, or if there is no significant proteasome-inhibitor sensitive increase in LANA1ΔSRD, this will suggest that DRiP processing is not directly inhibited by LANA1, increasing the likely importance of mature protein degradation to immune escape mechanisms.

### Example 10

### Effect of Codon Usage on the LANA1 SRD

No mechanism for synthesis-inhibition by the GAr has been reported. One possibility is that rare codon usage stalls polypeptide synthesis on the ribosome. If this is the case, re-engineering the LANA1 SRD to use common codons may increase the rate of synthesis without changing the primary sequence. If a cis-acting SRD is identified, codon optimization can be performed on the SRD motif using the method of Seyfang and Jin (Seyfang, A., and J. H. Jin. 2004. Multiple site-directed mutagenesis of more than 10 sites simultaneously and in a single round. Anal Biochem 324:285-91). Mutagenesis of multiple codons (>10) in the SRD to optimal human cell codon usage can be readily performed using this technique. The optimized SRD can be re-cloned into full-length LANA1 and the coding sequence verified by sequencing. Codon-optimized LANA1 (LANA-CO) can be first examined in vitro using de-coupled in vitro translation to determine if this improves LANA1 translation rate. Wild-type LANA1 and LANA1-CO can be transfected into 293 cells. If codon usage affects LANA1 synthesis, increased steady-state accumulation of CO-LANA1 compared to LANA1 can be seen by immunoblotting.

It is anticipated that if the SRD is due to suboptimal codon usage, then in vitro rates of translation will be greater for CO-LANA1 than wild-type LANA1. If neither is seen, then we can effectively rule out codon usage can be effectively ruled out as a mechanism for the LANA1 SRD. If, on the other hand, codon optimization of the SRD does significantly improve LANA1 peptide synthesis, we could directly measure whether or not this increases LANA1 DRiPs. Wild-type LANA1 and the LANA1-CO can be transfected into cells, untreated or treated with MG132, metabolically labeled with [³⁵S]-methionine and then fractionated. The DRiP fraction can be isolated at different time points, solubilized in Triton-X, TCA precipitated onto glass fiber filters and measured by scintillation counting. The codon-optimized version of LANA1 then can be examined as an immunogen to see if simple modification of the rate of synthesis affects MHC Class I restricted CD8⁺ T cell responses.

### Example 11

### Enhanced Immune Reactivity of Modified LANA1 Proteins

Regulation of CTL responses by LANA1 DI and/or SR domains can be measured in two ways: 1) By comparing modified LANA1, lacking DI and SR domains (LANA1ΔDI/SR), to wild-type LANA1 for their ability to elicit CD8⁺ T cell responses; and 2) by cloning LANA1-associated DI and SR domains into heterologous chicken ovalbumin (OVA) and monitoring their effects on CTL specific responses in vitro to the dominant H2-Kb restricted SIINFEKL (SEQ ID NO: 23) OVA epitope.

The premise of this experiment is that eliminating the effects of DI and/or SR domains will result in a greater quantity of LANA1 available for inducing a specific response, or for epitope presentation in the context of MHC Class I determinants. Similarly, if LANA1 destabilization is achieved in the experiments described above, codon optimization of LANA1 results in increased LANA1 DRiP formation, they can be examined in the same way as described below for DI/SR deletion constructs as additional experimental variations.

After eliminating the degradation inhibition (“DI”) and/or synthesis retardation (“SR”) domains, the effects on immune responses to LANA1 epitopes can be assessed. Once the DI/SR domain is defined, sequence-confirmed constructs of LANA1 and LANA1ΔDI/SR can be stably expressed in the P815 (H2d) cell line. This cell line has proven suitable for expression of a variety of genes using either plasmids or viral constructs (Miller, L., G. Alber, N. Varin-Blank, R. Ludowyke, and H. Metzger. 1990. Transmembrane signaling in P815 mastocytoma cells by transfected IgE receptors. J Biol Chem 265:12444-53; Prell, R. A., and N. I. Kerkvliet. 1997. Involvement of altered B7 expression in dioxin immunotoxicity: B7 transfection restores the CTL but not the autoantibody response to the P815 mastocytoma. J Immunol 158:2695-703 and Shelburne, C. P., and T. F. Huff. 1999. Inhibition of kit expression in P815 mouse mastocytoma cells by a hammerhead ribozyme. Clin Immunol 93:46-58). We can use previously reported methods for immunization of Balb/c mice (Moore, M. W., F. R. Carbone, and M. J. Bevan. 1988. Introduction of soluble protein into the class I pathway of antigen processing and presentation. Cell 54:777-85) and measure responses using both analyses of cytolytic function and IFNγ production by CD8⁺ T cells (Giezeman-Smits, K. M., H. Okada, C. S. Brissette-Storkus, L. A. Villa, J. Attanucci, M. T. Lotze, I. F. Pollack, M. E. Bozik, and W. H. Chambers. 2000. Cytokine gene therapy of gliomas: induction of reactive CD4⁺ T cells by interleukin-4-transfected 9 L gliosarcoma is essential for protective immunity. Cancer Res 60:2449-57 and Okada, H., J. Attanucci, K. M. Giezeman-Smits, C. Brissette-Storkus, W. K. Fellows, A. Gambotto, L. F. Pollack, K. Pogue-Geile, M. T. Lotze, M. E. Bozik, and W. H. Chambers. 2001. Immunization with an antigen identified by cytokine tumor vaccine-assisted SEREX (CAS) suppressed growth of the rat 9 L glioma in vivo. Cancer Res 61:2625-31).

To evaluate induction of T cell responses, mice can initially be immunized twice (day 0 and day 10) with transfected cells and will harvest splenocytes on day 17; the spleen can be disrupted to generate a single cell suspension; positive selection can be carried out for CD8⁺ T cells using MACS magnetic beads (Miltenyi) loaded with anti-CD8⁺ and then T cells can be cultured for 5 days on peptide pulsed (1 μM), irradiated (2000 RAD) splenocytes in a T25 flasks (Corning) containing 5 ml RPMI (RPMI 1640 with 25 mM HEPES supplemented with 10% FBS, 2 mM L-glutamine, 1 mM sodium pyruvate, 0.1 mM nonessential amino acids, 50 mM β-mercaptoethanol, 50 U/ml penicillin, and 50 μg/ml streptomycin) as previously described (Giezeman-Smits, K. M., H. Okada, C. S. Brissette-Storkus, L. A. Villa, J. Attanucci, M. T. Lotze, I. F. Pollack, M. E. Bozik, and W. H. Chambers. 2000. Cytokine gene therapy of gliomas: induction of reactive CD4⁺ T cells by interleukin-4-transfected 9 L gliosarcoma is essential for protective immunity. Cancer Res 60:2449-57). At the end of culture, viable T cells can be separated on Ficoll-Paque, and assayed for lytic activity against peptide pulsed P815 target cells or peptide pulsed (1-10 μM) Jurkat cells transfected to express H2 K^{d} in standard 4 hr, ⁵¹Cr-release assays at varying effector cell:target cell ratios (1:1, 2:1, 5:1, 10:1, 25:1) as previously described (Chambers, W. H., M. E. Bozik, S. C. Brissette-Storkus, P. Basse, E. Redgate, S. Watkins, and S. S. Boggs. 1996. NKR-P1⁺ cells localize selectively in Rat 9 L gliosarcomas but have reduced cytolytic function. Cancer Res 56:3516-25; Okada, H., T. Tsugawa, H. Sato, N. Kuwashima, A. Gambotto, K. Okada, J. E. Dusak, W. K. Fellows-Mayle, G. D. Papworth, S. C. Watkins, W. H. Chambers, D. M. Potter, W. J. Storkus, and I. F. Pollack. 2004. Delivery of interferon-alpha transfected dendritic cells into central nervous system tumors enhances the antitumor efficacy of peripheral peptide-based vaccines. Cancer Res 64:5830-8 and Yang, T., T. F. Witham, L. Villa, M. Erff, J. Attanucci, S. Watkins, D. Kondziolka, H. Okada, I. F. Pollack, and W. H. Chambers. 2002. Glioma-associated hyaluronan induces apoptosis in dendritic cells via inducible nitric oxide synthase: implications for the use of dendritic cells for therapy of gliomas. Cancer Res 62:2583-91). The relative capacity of T cells from immunized mice to lyse P815 expressing wild-type LANA1 vs. LANA1ΔDI/SR can be evaluated in standard [⁵¹Cr]-release assays which we perform routinely (Chambers, W. H., M. E. Bozik, S. C. Brissette-Storkus, P. Basse, E. Redgate, S. Watkins, and S. S. Boggs. 1996. NKR-P1⁺ cells localize selectively in Rat 9 L gliosarcomas but have reduced cytolytic function. Cancer Res 56:3516-25). As an indicator of relative numbers of specific T cells, we can calculate LU20/106 T cells comparing populations of T cells from animals immunized with wild type LANA1 vs. LANA1ΔDI/SR as previously described (Chambers, W. H., M. E. Bozik, S. C. Brissette-Storkus, P. Basse, E. Redgate, S. Watkins, and S. S. Boggs. 1996. NKR-P1+ cells localize selectively in Rat 9 L gliosarcomas but have reduced cytolytic function. Cancer Res 56:3516-25).

Using predictive algorithms for instance, RANKPEP or the SYFPEITHI database (www.syfpeithi.de), to predict LANA1 peptides likely to be presented in the context of mouse MHC Class I determinants, three peptides show strong potential for binding to H2-Kd (no LANA1 peptide reactivity was predicted for H2-K^{b}):

| | | |
|---|---|---|
| A M L V L L A E I | (Score 22) | (SEQ ID NO: 24) |
| S S P Q G P S T L | (Score 21) | (SEQ ID NO: 25) |
| L A P S T L R S L | (Score 20) | (SEQ ID NO: 26) |

Epitopes can be synthesized using an automated solid-phase peptide synthesizer (Applied Biosystems, Foster City, Calif.), purified (to greater than 95%) by reverse phase HPLC, and characterized for amino acid sequences by mass spectrometry. Each peptide may be evaluated for specific reactivity of T cells in 51Cr release cytotoxicity assays as described above.

As an additional comparison of responses to wild-type LANA1 vs. LANA1ΔDI/SR, T cell production of IFNγ can be evaluated using ELISPOT assays. Following the immunization schedule and in vitro restimulation protocols detailed above, LANA1-specific T cells will be generated in mice immunized with wild type LANA1 or LANA1 ΔDI/SR.

For ELISPOT assays, 96-well plates (Millipore) may be coated with 10 μg/ml of anti-mouse IFN-γ mAb in PBS overnight at 4° C. Unbound antibody can be removed by four successive washes with PBS. After blocking the plates with RPMI1640/10% mouse serum (1 h at 37° C.), 10⁵ CD8⁺ T cells and P815 cells (2×10⁴ cells) can be seeded into wells. Synthetic peptides (stocks at 1 mg/ml PBS) can then be added to appropriate wells at a final concentration of 10 μg/ml. Negative peptide control wells contained CD8⁺ T cells with P815 cells pulsed with irrelevant peptide, with P815 cells alone serving as an additional background control. Positive controls will be T cells plated with 5 μg/ml PHA. Cells can be suspended in a final volume of 200 μl/well. Plates can be incubated at 37° C. in 5% CO₂ for 24 h in the case of IFN-γ ELISPOT assays. After incubation, supernatants of culture wells can be harvested for ELISA analyses, and plates washed with PBS/0.05% Tween 20 (PBS/T) to remove cells. Captured cytokine can be detected at sites of its secretion by incubation for 2 h with biotinylated mAb anti-mouse IFN-γ at 2 μg/ml in PBS/0.5% BSA. Plates can then be washed six times with PBS/T, and avidin-peroxidase complex (diluted 1:100; Vectastain Elite Kit; Vector Laboratories) can be added for 1 h. Unbound complex can be removed by three successive washes with PBS/T and three rinses with PBS alone. AEC substrate (Sigma-Aldrich) can be added and incubated for 5 min for the IFN-γ ELISPOT assay. All determinations can be performed in triplicates, with spots imaged using a Zeiss AutoImager (and statistical comparisons determined using a Student two-tailed t test analysis). The data can be represented as mean IFN-γ spots per 100,000 CD8⁺ T cells analyzed.

In these experiments, we anticipate observing that cells expressing LANA1ΔDI/SR will have a greater capacity to induce MHC Class I-restricted responses among T cells than will cells expressing wild-type LANA1. This may be observed both in terms of enhanced levels of CTL activity, as well as enhanced numbers of T cells producing IFNγ. This, in combination with the evidence indicating that elimination of the DI/ST domain results in increased synthesis and degradation of this protein, will suggest a useful strategy for enhancing immune reactivity to a viral vaccine. Similarly, increased effector function against target cells expressing LANA1 ΔDI/SR in comparison to those expressing wild-type LANA1 will suggest better processing of LANA1 for generation of MHC Class I presented peptides.

As an alternative, especially if initial immunization protocols prove ineffective in generating substantial T cell responses, dendritic cells (DCs), loaded with lysates or eluted peptides of P815 cells transfected with LANA1 or LANA1ΔDI/SR can be used for immunizations. For experiments with pulsed DCs, immature DCs (iDCs) can be produced from Balb/c mice. As an additional means of quantifying LANA1-specific CTL, MHC class I tetramers bearing the predicted LANA1 peptides can be used to stain and quantify antigen-specific cells. Cells obtained ex vivo from the spleens and lymph nodes of sacrificed mice and from cell culture can be incubated with FITC-labeled anti-CD8 and PE-OVA tetramers.

In addition to the above, flow cytometric analyses of intracellular cytokine staining may be employed on both freshly isolated and restimulated (Day 5) CD8⁺ T cells from immunized mice cultured for 0, 1, 4, 24 and 48 hr with peptide pulsed [1-10 μM] P815 target cells or DCs. T cells can be washed in washing buffer (PBS containing bovine serum albumin (BSA; 0.5%) and sodium azide (NaN₃; 0.1%), and then permeabilized with FACS Permeabilizing Solution (BD) for 10 min, washed again and stained with fluorochrome conjugated monoclonal antibodies to surface molecules CD3 or CD8, as well as to the following intracellular cytokines: IFN-γ, TNFα, IL-2, IL-4, IL-10 or IL-13. Finally, the cells can be fixed with paraformaldehyde (PFA; 1%) and kept in the dark at +4° C. until analyzed. Cells can be gated based upon expression of CD3; and then two parameter histograms based upon staining with anti-CD8 and cytokines can be generated for each experimental condition. These protocols for flow cytometric measurement of intracellular cytokines are robust and are performed routinely.

### Example 12

### Heterologous Expression of LANA DI/SRD in Chicken Ovalbumin Construct

The following is an alternative approach for defining the effects of the DI and SR domains on immune reactivity to a defined T cell epitope in an artificial, but rigorously defined model system. That is, the generation and expression of T cell responses to, for example and without limitation, the SIINFEKL (SEQ ID NO: 23) epitope associated with chicken ovalbumin presented in the context of H2-Kb. This may initially be approached as simply an in vitro model system in which EL4 cells are transfected to express unmodified chicken ovalbumin (e.g. E.G7-OVA) or to express an ovalbumin construct containing the LANA1-associated DI/SR domain (OVA-DI/SR fusion protein). As a first experiment, CD8⁺ T cells can be isolated using negative selection on magnetic beads from OT-1 transgenic mice (C57B1/6-Tg(TcraTcrb) 1100 Mjb/J; Jackson Laboratory) which express a T cell receptor as transgene specific for the SIINFEKL (SEQ ID NO: 23) epitope of ovalbumin presented in the context of H2-K b. These T cells can be assayed for lytic activity against E.G7-OVA in comparison to EL4 expressing OVA-DI/SR fusion protein in standard, 4 hr ⁵¹Cr-release assays. A lesser capacity to mediate lysis of OVA-DI/SR fusion protein expressing EL4 cells in comparison to E.G7-OVA will suggest that there is a reduced level of expression and processing of OVA that is regulated by the LANA1 DI/SR domain. ELISPOT assays examining production of IFNγ by CD8⁺ T cells from OT-1 mice can be set up as described above, but using E.G7-OVA cells vs. EL4 expressing OVA-DI/SR fusion protein for comparison.

These experiments represent a rapid and simple means of assessing the effects of LANA-DI/SR on expression of a defined OVA epitope in the context of MHC Class I. However, it is possible that there will be differences in the transfected EL4 cell lines in terms of Class I expression that could influence these results. To account for this possibility, relative levels of expression of Kb may be determined using either flow cytometry or Western blot analyses. An additional concern is whether there will be a complete or partial reduction in the expression and processing of OVA in OVA-DI/SR fusion protein transfectant. If there is only a partial reduction, it is possible that the abundance of SIINFEKL (SEQ ID NO: 23) specific T cells in the OT-1 transgenic model will allow efficient recognition of target cells without a total loss of OVA expression and processing. If preliminary experiments detailing the expression and processing of OVA in OVA-DI/SR fusion protein transfectants suggests that there is some expression of OVA and there is not a clear difference in the ability of OT-1 CD8⁺ T cells to lyse this tumor in comparison to E.G7 cells, these experiments may be approached from another angle. That is, C57B1/6 mice immunized with either E.G7 or with EL4 expressing the OVA-DI/SR fusion protein can be used. This can be attempted initially with irradiated tumor cells, but if there is not a robust response, DCs loaded with lysates of E.G7 or EL4 expressing the OVA-DI/SR fusion protein can be used as described above. Following immunization, CD8⁺ T cells can be isolated from among splenocytes of the two groups of mice and assay their capacity for lysis of RMA-S (H2b) pulsed with SIINFEKL (SEQ ID NO: 23) in standard 4 hr ⁵¹Cr-release assays. Cytotoxicity assays can be carried out using freshly isolated CD8⁺ T cells and using CD8⁺ T cells following restimulation in vitro by SIINFEKL (SEQ ID NO: 23) peptide pulsed RMA-S cells. Quantitation of specific CTLs derived from mice immunized with E.G7 or EL4 expressing the OVA-DI/SR fusion protein can be carried out as described above.

The relative efficiency in inducing T cells producing IFNγ will be evaluated using ELISPOT assays. For ELISPOT assays, 96-well plates (Millipore) can be coated with 10 μg/ml of anti-mouse IFN-γ mAb in PBS overnight at 4° C. Unbound antibody can be removed by four successive washes with PBS. After blocking the plates with RPM11640/10% mouse serum (1 h at 37° C.), 10⁵ CD8⁺ T cells and P815 cells (2×10⁴ cells) can be seeded into wells. Synthetic peptides (stocks at 1 mg/ml PBS) can then be added to appropriate wells at a final concentration of 10 μg/ml. Negative peptide control wells contained CD8⁺ T cells with P815 cells pulsed with irrelevant peptide, with P815 cells alone serving as an additional background control. Positive controls can be T cells plated with 5 μg/ml PHA. Cells can be suspended in a final volume of 200 μl/well. Plates can be incubated at 37° C. in 5% CO₂ for 24 h in the case of IFN-γ ELISPOT assays. After incubation, supernatants of culture wells can be harvested for ELISA analyses, and plates washed with PBS/0.05% Tween 20 (PBS/T) to remove cells. Captured cytokine can be detected at sites of its secretion by incubation for 2 h with biotinylated mAb anti-mouse IFNγ at 2 μg/ml in PBS/0.5% BSA. Plates can then be washed six times with PBS/T, and avidin-peroxidase complex (diluted 1:100; Vectastain Elite Kit; Vector Laboratories) can be added for 1 h. Unbound complex can be removed by three successive washes with PBS/T and three rinses with PBS alone. AEC substrate (Sigma-Aldrich) can be added and incubated for 5 min for the IFNγ ELISPOT assay. All determinations can be performed in triplicates, with spots imaged using the Zeiss AutoImager (and statistical comparisons determined using a Student two-tailed t test analysis). The data can be represented as mean IFNγ spots per 100,000 CD8⁺ T cells analyzed.

It is anticipated that the DI/SR domain of LANA1, when fused with OVA, will cause a marked decrease in expression and processing of OVA peptides in EL4 cells. Further, this change in production of OVA epitopes is expected to have a powerful regulatory influence on the capacity of T cells to mediate effector functions in vitro against the tumor cells (efferent effector function) and to result in a reduced capacity to induce specific T cells in vivo.

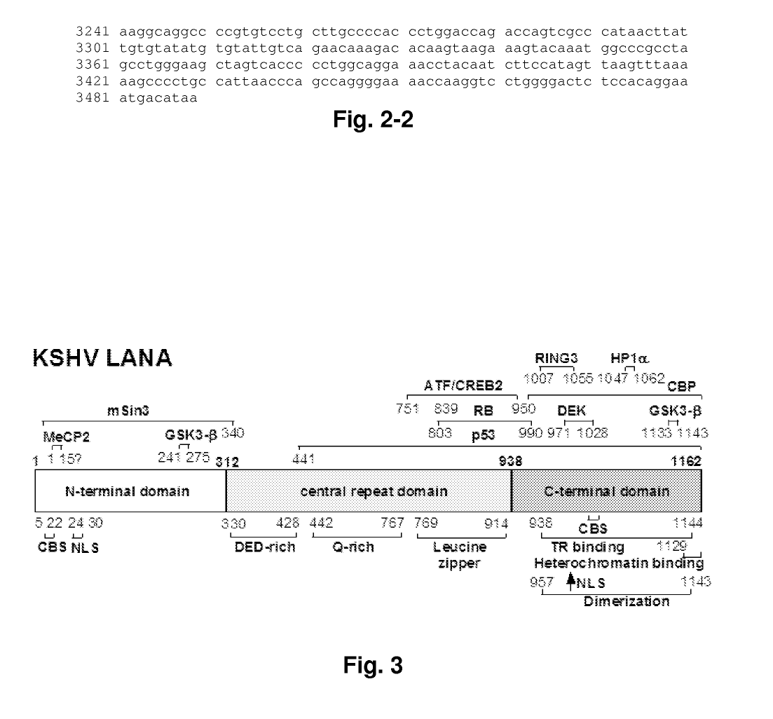

OVA was selected for these studies because it is a strong and well-characterized antigen. Although it might be expected that OVA antigenicity would reduce our ability to measure a response, the opposite has been found experimentally using GAr-OVA fusion proteins (see FIG. 3 in Yin, Y., B. Manoury, and R. Fahraeus. 2003. Self-inhibition of synthesis and antigen presentation by Epstein-Barr virus-encoded EBNA1. Science 301:1371-4). The reason for this is that even partial inhibition of strong antigenic responses against OVA are readily detected as significant reductions in response. Use of a weak antigen would have a smaller range of possible change when fused to the LANADI/SR domain, making detection of an effect more difficult to measure.

### Example 13

### LANA1 Heterologous Constructs

LANA1 heterologous constructs containing N, C-terminus or the central repeat domain and their fusions were generated using PCR and BC-1 KSHV strain DNA (sequence from Russo PNAS paper which was also deposited in GenBank). Two forward primers were designed that contained either EcoRI or EcoRV restriction sites, and reverse primers contain either EcoRV or HindIII sites. These sites were used for cloning into the vector (see, Table I). PCR components were high fidelity PCR buffer (60 mMTris-SO₄/pH8.9, 18 mM ammonium sulfate), 3 mM MgSO₄, 2U Taq high fidelity DNA polymerase (Invitrogen, Carlsbad, Calif., USA), 0.4 mM dNTPs and 1 pmol of forward and reverse primers. The PCR cycling profile was 94° C. for 2 min; 94° C. for 1 minute (1′), 60° C. for 1′, 72° C. for 1.5′ (30×); 72° C. for 10′. 5% DMSO and 1.5M betaine were added for the central repeat domain between 1288 bp and 2304 bp (CR2), and for region between 2305 bp and 2817 bp (CR3) the reaction was performed with 5% DMSO.

| | | |
|---|---|---|
| LANA1 | | |
| Region | Primers forward | Primers reverse |

| | | |
|---|---|---|
| N-terminus | GAATCCATGGCGCCCCCGG | CCGATATCCTTATTGTCAT |
| | GAATGCGC | TGTCATCCTT |
| | (SEQ ID NO: 27) | (SEQ ID NO: 28) |
| C-terminus | CGATATCATCTTGCACGGG | CCAAGCTTTGTCATTTCCT |
| | TCGTCATCC | GTGGAGAGTC |
| | (SEQ ID NO: 29) | (SEQ ID NO: 30) |
| CR1 | CCGAATTCGACAAGGATGA | CGATATCGCTCAACGTTTT |
| | CAATGACAAT | GTTTCCATCG |
| | (SEQ ID NO: 31) | (SEQ ID NO: 33) |
| | CCGATATCGACAAGGATGA | |
| | CAATGACAAT | |
| | (SEQ ID NO: 32) | |
| CR2 | GAATTCGGCGATGGAAACA | GATATCCTCCTGCTCCTGC |
| | AAACGTTGAGC | TCCTCCTGCT |
| | (SEQ ID NO: 34) | (SEQ ID NO: 36) |
| | GATATCGGCGATGGAAACA | |
| | AAACGTTGAGC | |
| | (SEQ ID NO: 35) | |
| CR3 | GAATTCTTAGAGGAGCAGG | GATATCCAAGATTATGGGC |
| | AGCAGGAGTTA | TCTTCCACCGT |
| | (SEQ ID NO: 37) | (SEQ ID NO: 39) |
| | GATATCTTAGAGGAGCAGG | |
| | AGCAGGAGTTA | |
| | (SEQ ID NO: 38) | |

The PCR product was analyzed in 1.0% agarose gel stained with ethidium bromide. The band was isolated following the protocol for QIAquick gel extraction kit (Qiagen, Valencia, Calif.). TOPO Clone pCR2.1 (Invitrogen, Carlsbad, Calif.) using one third of the reaction suggested by the company was used for cloning. Transformation was performed using one shot TOP10 chemical competent cells (Invitrogen, Carlsbad, Calif.), using 16 μL of cells for each 2.66 μL of ligation product. After that, the cells were kept in ice for 30 minutes, and the heat shock has been done for 30 seconds at 42° C. The samples were kept in ice for 2 minutes and then 200 μL of LB Broth were added. The samples were shaken for one hour at 37° C., and they were plated in LB-ampicillin plates pre-treated with 1.6 mg of 5-bromo-4-cloro-3-indolil-β-D-galactopyranoside (X-gal). The plates were incubated at 37° C. overnight. Three to four white colonies were picked, and each one was incubated in shaker at 37° C. overnight in 3 mL of liquid media LB broth with 0.1 μg/μL of ampicillin. The plasmid DNA purification was performed following QIAprep spin miniprep kit and a microcentrifuge (Qiagen, Valencia, Calif.). Digestions were performed using 3 U of EcoRI enzyme, 10× NEbuffer EcoRI (50 mM NaCl, 100 mM Tris-HCl, 10 mM MgCl₂, 0.025% Triton X-100-pH 7.5 at 25° C.), and 1-2 μg of DNA for 1 hour at 37° C. The results were analyzed in a 1.0% agarose gel stained with ethidium bromide. The clones positive for each region were digested using the same enzymes used to cut the primers. 10-20 μg of DNA were cut with EcoRI/EcoRV, EcoRV/HindIII, or just EcoRV, using 10-25 U. The buffers used were 10× NEbuffer EcoRI (50 mM NaCl, 100 mM Tris-HCl, 10 mM MgCl₂, 0.025% Triton X-100-pH7.5 at 25° C.), 10×NEbuffer 2 (50 mM NaCl, 10 mM Tris-HCl, 10 mM MgCl₂, 1 mM dithiothreitol-pH 7.9 at 25 C), and 10×NEbuffer 3 (100 mM NaCl, 50 mM Tris-HCl, 10 mM MgCl₂, 1 mM dithiothreitol-pH 7.9 at 25° C.) respectively. The digestions were performed at 37° C. overnight. The vector, pCMV Tag2B (Stratagen, La Jolla, Calif.), was digested with these same enzymes, following the same protocol. All enzymes are presented as a unique site in this vector. The gel extraction was performed following the same protocol cited before, and the ligation was performed using 400 U of T4 DNA ligase (New England Biolabs, Beverly, Mass.), 1×T4 DNA ligase reaction buffer (50 mM Tris-HCl (pH7.5), 10 mM MgCl₂, 10 mM dithiothreitol, 1 mM ATP, 25 μg/ml bovine serum albumin), and 100 ng of vector and plasmid DNA overnight at 16° C. The transformation was performed following the same protocol for TOPO Clone, but this time using 50 μL of TOP10 competent cells and 5 μL of each ligation product, and they were plated in kanamycin plates, without X-gal. Three to four colonies were picked and incubated overnight at 37° C. in liquid LB Broth with kanamycin (0.05 μg/μl). The plasmid DNA was purified following the same protocol that we have cited before. The digestion was performed with the same protocol from TOPO Clone, but this time using the specific restriction enzymes. The digestion was checked in 1.0% agarose gel stained with ethidium bromide, and the positive samples were sequenced.

These constructs were sub-cloned as follows. The vector pCMV Tag2B was modified by PCR in its multiple cloning site to add two methionine residues (ATGs) 5′ to the EcoRI site, and the constructs described above, with wild type pCMV Tag2B, were digested with EcoRI/EcoRV or EcoRI/HindIII for sub-cloning into the ATG-pCMV Tag2B, following the protocols described above. For the clones fused with GFP, a PCR was performed to insert BamHI and EcoRI site in GFP using pEGFP-C1 (broadly-available) as a template and primers 5′ CATGGATCCGCCACCATGGTGAGCAAGGGC 3′ (SEQ ID NO: 40) and 5′ CTTGAATTCCTTGTACAGCTCGTCCATGC 3′ (SEQ ID NO: 41). After digestion, this product was fused in the pCMVs Tag2B constructs, resulting in GFP in the N-terminus. For the C-terminus GFP constructs, the same GFP source was used as a template, but the primers contained HindIII and XhoI sites (5′ CATAAGCTTGTGAGCAAGGGCGAGGAGCTG3′ (SEQ ID NO: 42) and 5′ CTTGAATTCCTTGTACAGCTCGTCCATGC 3′ (SEQ ID NO: 41), was fused with the constructs in pCMVTag2B.

In vitro translation for synthesis inhibition experiments were performed using [³⁵S]-methionine. LANA1 C-terminal truncations were generated using restriction enzymes sites: HincII (963 bp), Ac1I(1303 bp), SbfI (1391 bp, 1448 bp, 1490 bp), BsmbI (2785 bp), Nru (2942 bp) and XhoI. XhoI cuts in the vector for full-length LANA1 inserted. A second synthesis retardation experiment was performed using clones with different LANA1 domains fused with GFP either at the N- or the C-terminus. All in vitro transcription/in vitro translations were performed using Riboprobe in vitro transcription systems and Rabbit reticulocyte lysate system according to the protocol suggested by Promega, Madison, Wis. Equimolar amounts of RNA were used to perform the translation, using 2.5 pmol of RNA in the N-truncation experiment and 2.0 pmol of RNA in the synthesis inhibition experiments with LANA1 domains fused with GFP.

293 cells were cultured in DMEM media with 10% FBS (Life Technologies, Grand Island, N.Y.) and 2 mM L-glutamine. Transient transfections were performed with Lipofectamine 2000 (Invitrogen, Carlsbad, Calif.), and the cells were harvested during time points, 0, 4, 8, 12, 16, and 24 or 27 hours. The cells (1.7×10⁶) were lysed in 80-100 μL lysis buffer (50 mM Tris pH8.0, 150 mM NaCl, 3 mM EDTA, 1% Triton X-100, 10% glycerol, 1 mM NaF and 1 mM Na Orthovanadate), containing DTT (1 mM), PMSF (0.1 mM), aprotinin (1 μg/ul), leupeptin (1 μg/ul) and pepstatin (1 μg/ul), and were sonicated for 10 seconds. For GFP analysis, 50 μg of total protein in a total volume of 100 μl of PBS were analyzed using a fluorescence GFP reader.

Protein expression was analyzed by western-blot using the cells from the last time point. Protein extracts were resolved by 8% or 10% sodium dodecyl sulfate-polyacrylamide gel electrophoresis (SDS-PAGE) and transferred to nitrocellulose membranes (Schleicher and Schuell, Kneene, N.H., USA). After blocking at least 1 hour in TBS (50 mM Tris, 0.138M NaCl, 2.7 mM KC1, pH 8.0) supplemented with 5% skim milk and 0.05% Tween-20, the membranes were incubated with primary antibody mouse anti-GFP (Zymed, South San Francisco, Calif.) for 1 hour. Immunocomplexes were visualized using enhanced chemiluminescence (ECL, Amersham Pharmacia Biotech). After this analysis the gel was stripped and blotted with mouse anti-flag M2 monoclonal antibody (Sigma-Aldrich, St. Louis, Mo.) or rat monoclonal anti-ORF73 (Advanced Biotechnologies, Columbia, Md.).

### Results

### Synthesis Retardation

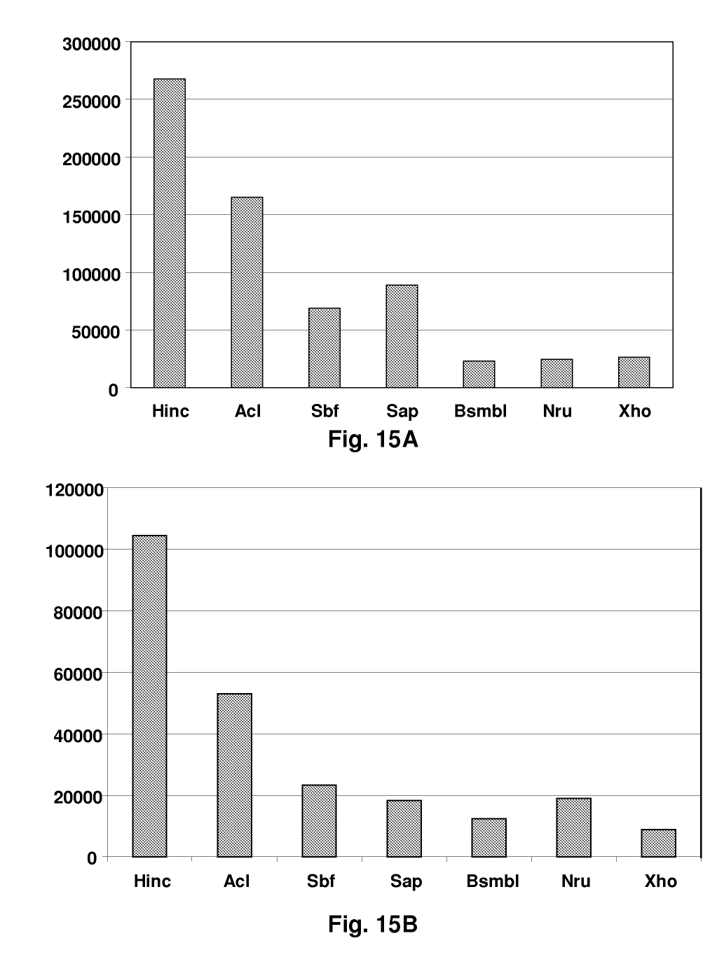

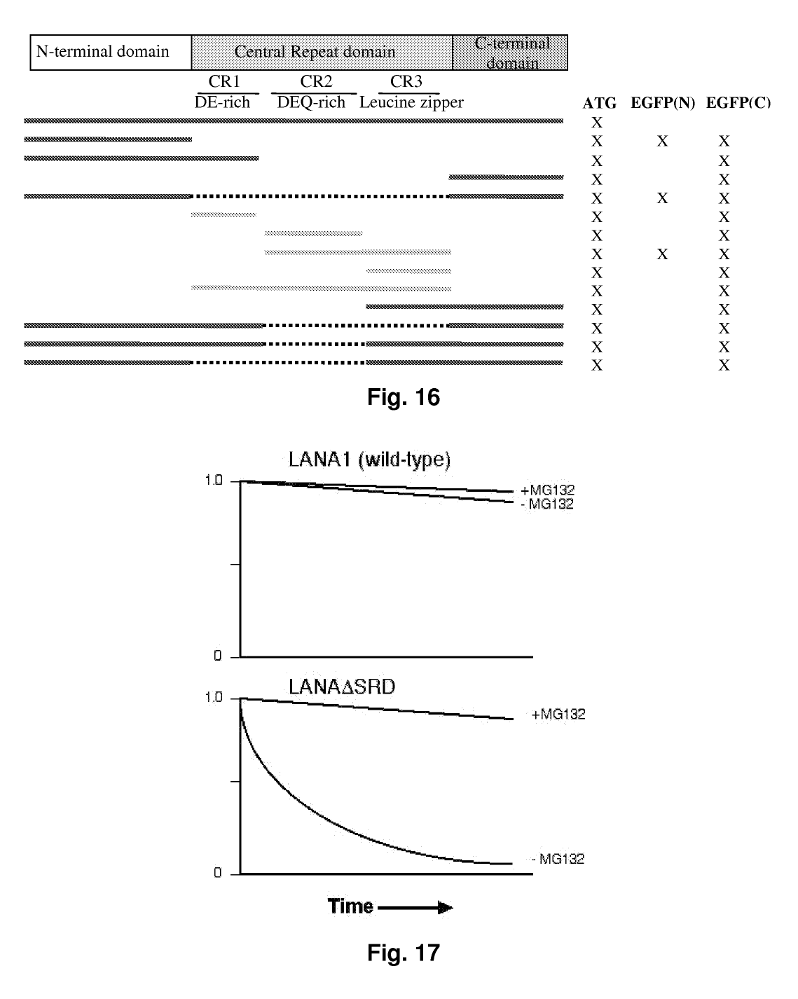

The C-truncations generated by restriction enzyme digest are presented in FIG. 18A. Analysis of in vitro transcription/translation for those constructs using [³⁵S]-methionine (FIG. 18B) indicates that two points in LANA1 are seen to be connected with a decrease in protein synthesis, the first one is located in the region between the Ac1I and SbfI cuts and the second between the SapI and BsmBI cuts, located in the beginning of the DEQ (aspartate-glutamate-glutamine) repeat region (CR2) and the leucine zipper region (CR3), respectively. To analyse if there is a decrease in the synthesis due to an amino acid deficit, we have repeated the in vitro translation using the amino acid mixture provided and added 1 mM of D, E and Q. FIGS. 19A and 19B demonstrate the quantitative analyses by phosphoimager for those reactions. The experiment shown in FIG. 19A used the amino acid mixture provided in the in vitro translation kit. In FIG. 19B, the same amino acid mixture was supplemented with D, E and Q. There is no significant difference between either reaction, indicating that the translation retardation occurs in cis, and is not likely caused by amino acid depletion during translation of the repeats.

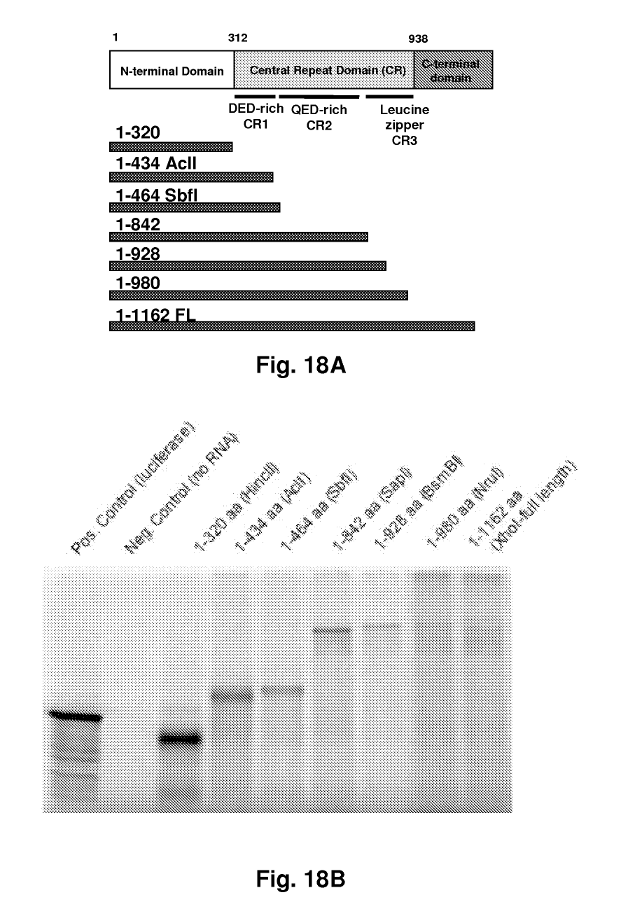

As shown above, the CR2/CR3 region appeared to be responsible for the synthesis retardation. Additional in vitro transcription/translation experiments were performed with the LANA1 N-terminus or CR2/CR3 domain fused with GFP at either the N- or C-terminus of the GFP in order to study if the position of GFP is important for the synthesis retardation function (FIG. 20). Although the size is higher than expected (should be 63 kDa, but is seen as a 85-90 kDa band, though an approximately 30 kDa increase in apparent size typically is seen with all LANA1 proteins in SDS-PAGE systems), product was generated from the LANA1 N-terminal constructs fused to GFP at either the N- or C-terminus. However, for the CR2/CR3 construct no band was seen (should be 83 kDa), demonstrating that synthesis retardation is caused by this domain.

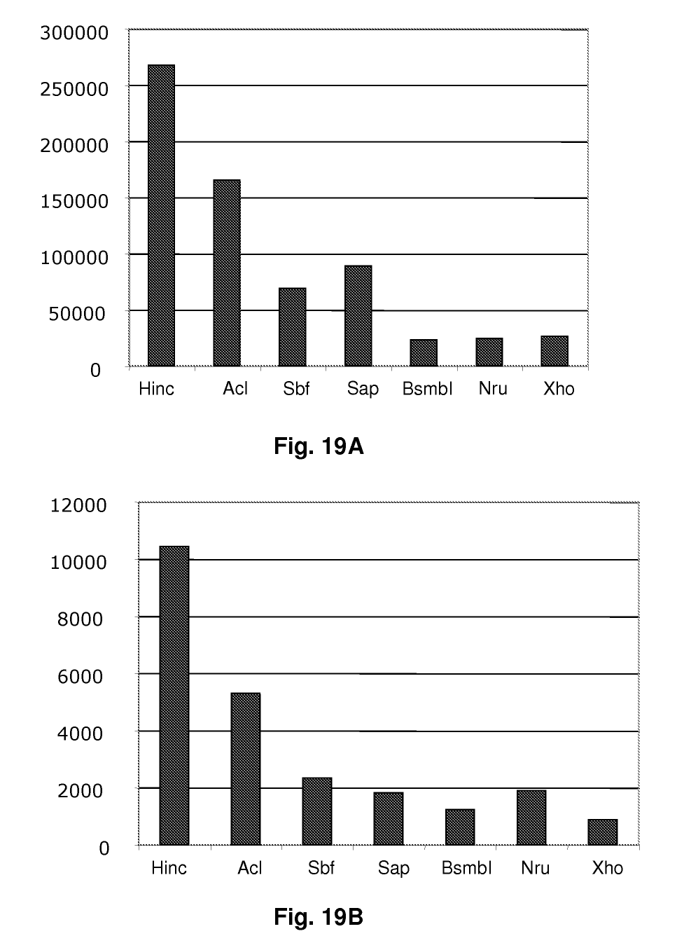

To analyze cis versus trans inhibition, an in vitro transcription was performed using the same amount of RNA for the construct with N and C fused, in which the central repeat domain is deleted. The amount of the construct that containing full-length LANA1 was increased (FIG. 21). This reaction was followed by in vitro translation, and we concluded that there is no trans inhibition by the full-length LANA1.

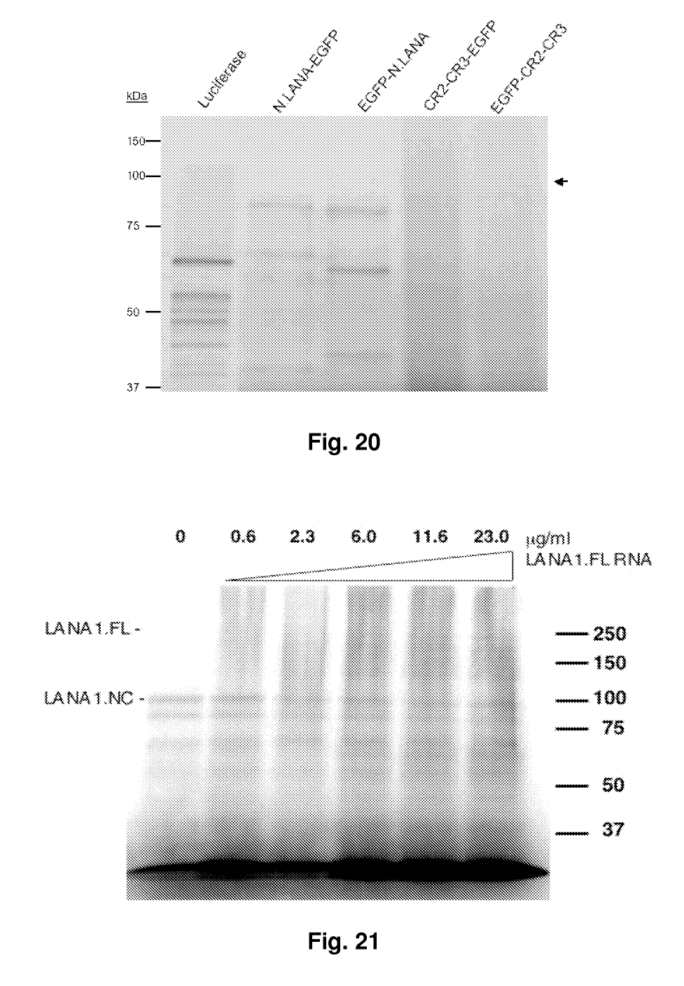

After analysis for protein expression by western-blot (data not shown), the cells transfected with different LANA1 constructs were analyzed by fluorescence for GFP. FIG. 22 shows that for CR1, CR2, CR3, and the fusion CR1-CR2 there is high levels of GFP as compared to vector alone (labeled “GFP vector”). A decrease in synthesis is seen with the fusion between CR2 and CR3 (labeled “GFP-CR2-3”). Based on the high levels of GFP measured for GFP-CR2 and GFP-CR3, the low level of GFP for GFP-CR2-3 suggested that the junction between CR2 and CR3 is responsible for retardation of protein synthesis. All GFP-LANA1 fusion constructs were cloned with GFP at the N-terminus. These data further illustrate that CR2/CR3 domain is responsible for the synthesis inhibition.

### Degradation Inhibition

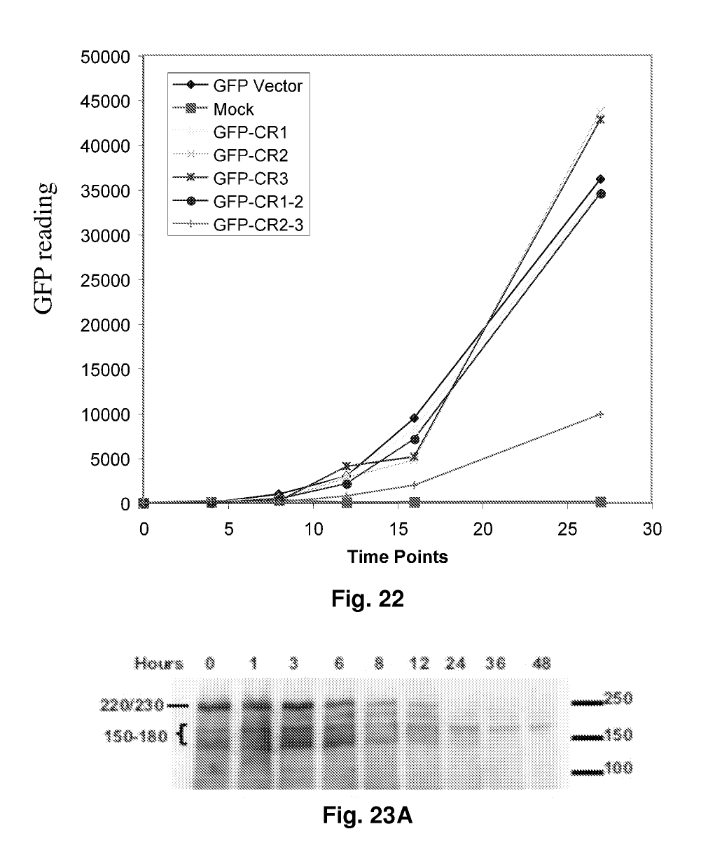

LANA 1 turnover in PEL cells was analyzed after treatment with cycloheximide, which prevents new protein synthesis, and the cells were harvested in 0, 1, 3, 6, 8, 12, 24, 36 and 48 hours after the treatment. FIG. 23A shows that the half life of LANA1 is 12-24 hours as compared to the half-life of the cellular protein IRF3 (FIG. 23B), which was about 3-6 hours. After 12 hours of treatment, a shift between the ˜220-230 kDa bands and 150-180 kDa bands was observed. It is not completely understood in the literature if these 150-180 kDa bands are degradation products or different isoforms of LANA1. Because the half-life of LANA1 was prolonged, the degradation inhibition capacity of the same central repeat region that causes synthesis inhibition was studied.

For this purpose, as described above, 293 cells were transfected with constructs comprising the LANA1 N-terminus, the LANA1 C-terminus, the LANA1 construct in which the central repeat domain was deleted (N- and C-terminus fused), and full length LANA1. FIG. 9B shows the results of this experiment and indicates cells treated (+) and untreated (−) with MG132, a proteasome inhibitor. LANA1 full length exhibits a minimal increase after treatment with MG132 when compared with other constructs, which accumulated dramatically. These data indicate that the central repeat region is responsible for the reducing of LANA1 turnover.

As also shown above, GFP de-stabilized (dsGFP), which has a half-life about 1 or 2 hours, was fused with LANA1 CR2/CR3. As shown in FIG. 11, 293 cells transfected with dsGFP had no signal 12 hours after cycloheximide treatment, but LANA1 CR2/CR3 inhibited turnover of dsGFP. These data confirm that the CR2/CR3 domain inside the central repeat region is responsible for turnover inhibition.

### Discussion

Major histocompatibility complex class I (MHC I) molecules typically bind to peptides generated by proteasomes from a cytosolic pool of proteins, and this will generate CD8⁺ T cell epitopes. Defective ribosomal products (DRiPs) are important source of antigenic peptides for MHC I molecules, they may be derived either from a premature termination product or as a misfolded protein. DRiPs are degraded by proteasomes, increasing the potential for generation of CD8⁺ antigens. These mechanisms are the two most important ways for MHC I CTL surveillance. This might explain why some intracellular pathogens may evolve specific mechanisms to minimize DRiP formation (Yewdell, J. W., Anton, L. C. and Bennink, J. R. 1996. Defective Ribosomal Products (DRiPs). A Major source of antigenic peptides for MHC class I molecules? J. Immunol. 157:1823-6; Pamer, E. and Cresswell, P. 1998. Mechamisms of MHC Class I-restricted antigen processing. Annu. Rev. Immunol. 16: 323-58; Rock, K. L. and Golderg, A. L. 1999. Degradation of cell proteins and the generation of MHC class I-presented peptides. Annu. Rev. Immunol. 17: 739-79 and Schubert, U., Anton, L. C., Gibbs, J., Norbury, C. C., Yewdell, J. W. and Bennink, J. R. 2000. Rapid degradation of a large fraction of newly synthesized proteins by proteasomes. Nature. 404: 770-4).

KSHV may avoid CTL surveillance through a number of mechanisms as a result of the diverse functions that some of its viral products. Interesting products encoded by ORFs K3 and K5 are expressed during lytic virus cycle, and may act increasing the internalization and endocytosis of MHC I, consequently avoiding that CTL recognition of actively-infected cells (Lorenzo, M. E.; Ploegh, H. L. and Tirabassi, R. S. 2001. Viral immune evasion strategies and the underlying cell biology. Semin. Immunol. 13: 1-9). Moreover, chemokines like vMIP I, II and III and cytokines, such as viral interleukin (vIL-6) polarizes immune response toward Th2 rather than Th1Schulz, T. F.; Sheldon, J. and Greensill, J. 2002. Kaposi's sarcoma associated herpesvirus (KSHV) or human herpesvirus 8 (HHV8). Virus Res. 82: 115-26 and Lindow, M., Nansen, A., Bartholdy, C., Stryhn, A., Hansen, N. J., Boesen, T. P., Wells, T. N., Schwartz, T. W. and Thomsen, A. R. 2003. The virus-encoded chemokine vMIP-II inhibits virus-induced tc1-driven inflamation. J. Virol. 77: 7393-400).

It is shown herein that the CR2/CR3 domains within the LANA1 central repeat domain are important for synthesis retardation and proteasome inhibition. Although there is no amino acid analogy between LANA1 and EBNA1, the latter has a central repeat region (GAr) responsible for inhibiting its own synthesis and proteosomal processing, and retarding mRNA translation. Both mechanisms cooperate in EBNA1 to avoid from MHC I CD8⁺ cell surveillance.

The in vitro experiments described herein indicate that the CR2/CR3 region and/or the region between the QED repeats of CR2 and/or the leucine zipper of CR3 are responsible for the synthesis retardation and degradation inhibition.

Although the EBNA1 GAr region appears to protect the antigen from proteasomal processing and restricts presentation to CD8⁺ cells, Lee et al. (2004) have show that CD8⁺ T cells specific for EBNA1 play important role in control of EBV infection, indicating that the presence of the GAr region and deletion thereof may be exploitable for therapeutic purposes (Lee, S. P., Brooks, J. M., Al-Jarrah, H., Thomas, W. A., Haigh, T. A., Taylor, G. S., Humme, S., Schepers, A., Hammerschmidt, W., Yates, J. L., Rickinson, A. B. and Blake, N. W. 2004. CD8 T cell recognition of endogenously expressed Epstein-Barr virus nuclear antigen 1. J. Exp. Med. 199: 1409-20, 2004).

As described by Yin et al. (2003, Self-inhibition of synthesis and antigen presentation by Epstein-Barr virus-encoded EBNA1. Science 301:1371-4), the GAr has an inhibitory effect on its own translation in cis. Using ovalbumin (OVA) fused to the Gar, the authors concluded that the gar retarded translation of OVA, reduced ova DRiP formation and reduced CTL responses against OVA Antigen.

The DRiP experiment, above, shows specifically that the central repeat domain, represented by full-length LANA1, was not affected by MG132 treatment as compared to N-terminus, C-terminus and N-terminus and C-terminus-fused domains. Likewise, fusion of the CR2/CR3 region to destabilized GFP inhibited turnover of that protein.

Fine-scale deletions also may be generated within the SR domain to localize specific sequences involved in synthesis retardation of the heterologous GFP fusion protein. If, for example, the CR2 repeat region is confirmed to retard synthesis, the region is composed of glutamine-rich variable repeat regions that can be examined individually (QQQEP (SEQ ID NO: 4), QQQREP (SEQ ID NO: 43) and QQQQDE (SEQ ID NO: 11)). Constructs containing 1, 4 and 8 repeat sequences can be generated, cloned as fusion proteins into GFP and tested for retardation of peptide synthesis to determine the effect of repeat length on synthesis retardation. Additional mutagenesis of individual residues in the repeat sequences may provide insight into mechanisms and biochemical rules (e.g. effect of charge, conformation, codon usage, etc.) for synthesis inhibition.

### Example 14

### Disruption of CR2/CR3 Junction

In light of the results above, several constructs were synthesized to determine whether the CR2/CR3 junction was critical to synthesis retardation and degradation inhibition, We disrupted the junction first by introducing a 6 base-pair EcoRI restriction endonuclease site as well as to clone in the CR1 region (115 aa sequence extending from aa 321 to aa 437 of LANA1). We additionally cloned the CR2/CR3 region in reverse orientation (3RC-2RC) and also in reverse order (CR3-CR2). These constructs will allow us to determine if either the junction or the primary RNA sequence of the region is critical in the inhibition of degradation and synthesis retardation.

### Example 15

### Localization of the Proteasome Inhibition (or Degradation Inhibition) Domain to CR2

Different regions of LANA1 were attached to a destabilized GFP that undergoes rapid proteasomal turnover. Using this method, fusion proteins of destabilized enhanced GFP were created with one of the following different regions of LANA1: CR1, CR2, CR3, CR2/CR3, or CR2/EcoRV/CR3, having an EcoRV restriction site, GATATC, inserted between the CR2 and CR3 regions. After treating the cells with cycloheximide to stop new protein synthesis, fluorescence was measured at time t=0 and at time t=12 hours after cycloheximide treatment. Higher values of fluorescence at 12 hours correlate with a more stable fusion protein.

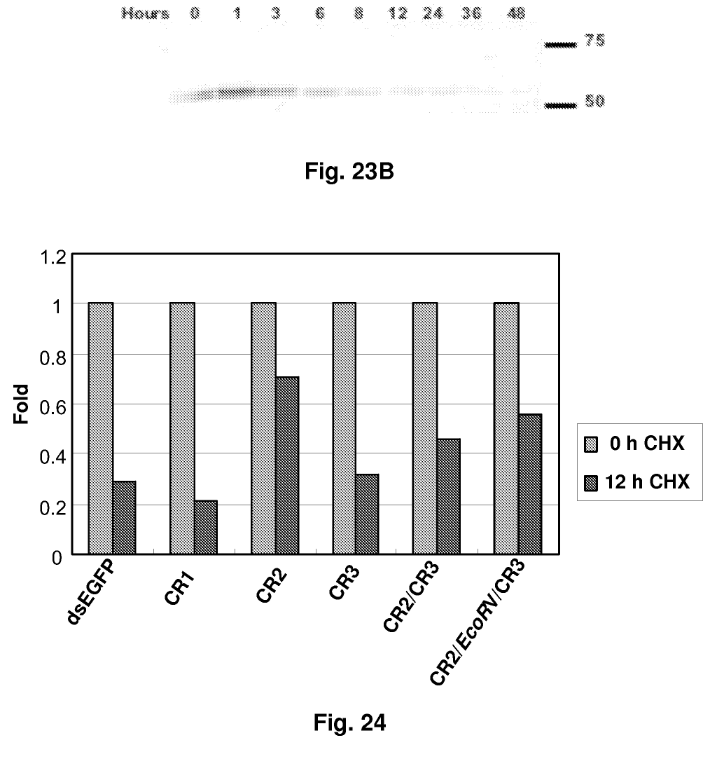

As shown in FIG. 24, the dsGFP-CR2 fusion protein was the most stable after 12 hours from cycloheximide treatment. Therefore, the CR2 region was the most effective in preventing proteasomal turnover compared to the other regions of LANA1. Deletion of CR2 will likely enhance cellular turnover of LANA1 and should generate a stronger immune response.

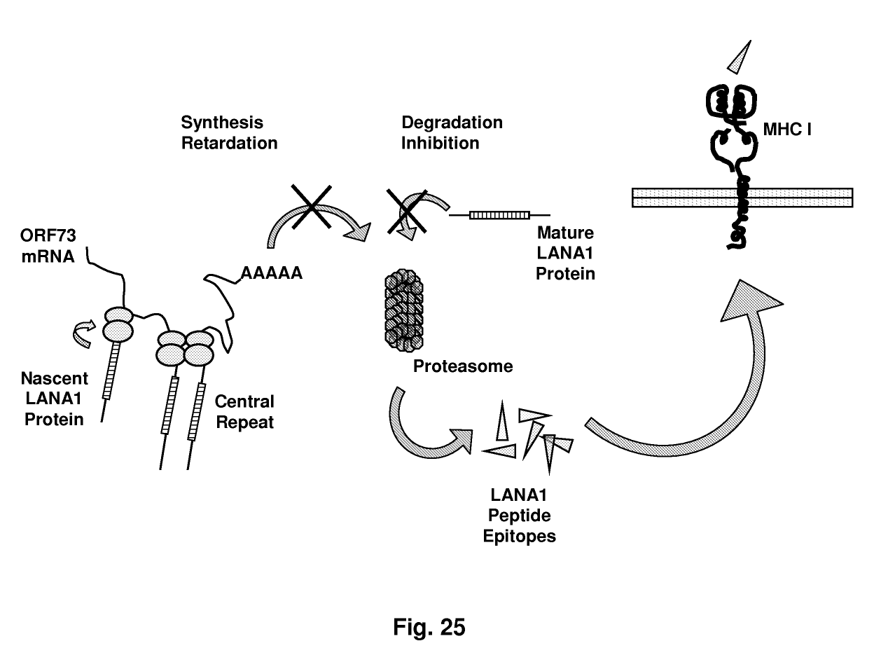

The above examples (such as in Example 5) show the primary translation retardation (or synthesis retardation or “SR”) domain to be the CR2/CR3 junction. The present example shows the primary proteasome inhibition (or degradation inhibition or “DI”) domain to be the CR2 region, though the CR2/CR3 junction appears to have inhibitory effects, too. Eliminating either or both domains should result in a more highly immunogenic protein. Without any intention of being bound by the following theories, FIG. 25 is a schematic showing two mechanisms of CTL immune invasion by the CR domains of LANA1. In the first mechanism, the synthesis retardation domain of LANA1 during initial translation of ORF73 mRNA slows down protein translation and thereby reduces misfolded protein turnover. In the second mechanism, the proteasome inhibition (or degradation inhibition or “DI”) domain slows down the degradation of mature LANA1 proteins by the proteosome. By these two distinct mechanisms, LANA1 is though to retard peptide processing and MHC-1 antigen presentation.

In Zaldumbide et al., the entire CR region (CR1+CR2+CR3, amino acids 360-911) was deleted (Zaldumbide, A., Ossevoort, M., Wiertz, E. J. H. J., and Hoeben R. C. 2007. In cis inhibition of antigen processing by the latency-associated nuclear antigen I of Kaposi sarcoma Herpes virus. Molec Immunol 44:1352-60). However, highly immunogenic domains may be present in CR1 and/or in CR3. As disclosed herein, the SR and DI domains are primarily localized within the CR2 region and the CR2/CR3 junction. By targeting the SR and/or DI domains more precisely, a more highly immunogenic peptide may be produced and a nearly full-length LANA1 polypeptide will be presented to MHC I.

We claim:

1. A method of eliciting an immune response to KSHV LANA1 in a patient comprising introducing into a cell of the patient an immunologically-enhanced LANA1 polypeptide (“ieLANA1 peptide”) for eliciting a CTL immune response to LANA1, comprising a LANA1 amino acid sequence comprising one or more LANA1 T-cell epitopes, and wherein the LANA1 amino acid sequence is modified to exhibit increased proteasomal degradation as compared to wild-type LANA1 protein.

2. The method of claim 1, wherein the ieLANA1 polypeptide does not comprise a portion of a LANA1 central repeat domain having the capacity to inhibit proteasomal degradation of a polypeptide or inhibit translation of a polypeptide attached in frame to that portion of the LANA1 central repeat domain, so that the ieLANA1 polypeptide exhibits increased proteasomal degradation as compared to wild-type LANA1 protein.

3. The method of claim 2, wherein the ieLANA1 polypeptide comprises the LANA1 amino acid sequence of SEQ ID NO: 1 in which a portion of the LANA1 central repeat domain having the capacity to inhibit proteasomal degradation of a polypeptide and inhibit translation of a polypeptide is modified to decrease the ability of that portion to inhibit proteasomal degradation of a polypeptide.

4. The method of claim 3, wherein the ieLANA1 polypeptide comprises the LANA1 amino acid sequence of SEQ ID NO: 1 in which from about amino acid 330 to about amino acid 938 are deleted.

5. The method of claim 4, wherein the ieLANA1 polypeptide comprises the LANA1 amino acid of SEQ ID NO: 1 in which from amino acid 330 to amino acid 938 are deleted.

6. The method of claim 2, wherein the ieLANA1 polypeptide comprises the LANA1 amino acid sequence in which a portion of one or both of CR2 and CR3 of the LANA1 amino acid sequence is modified to decrease the ability of that portion of one or both of CR2 and CR3 to inhibit proteasomal degradation of a polypeptide.

7. The method of claim 6, wherein the LANA1 amino acid sequence is modified such that one or both of CR2 and CR3 are substantially or completely deleted.

8. The method of claim 6, wherein the LANA1 amino acid sequence comprises the amino acid sequence of SEQ ID NO: 1 in which from about 100 to 509 amino acids of amino acids 428-938 are deleted, including a CR2/CR3 junction.

9. The method of claim 6, in which CR2 is substantially or completely deleted or replaced in the ieLANA1 polypeptide.

10. The method of claim 6, in which CR3 is substantially or completely deleted or replaced in the ieLANA1 polypeptide.

11. The method of claim 6, in which CR2 and CR3 are substantially or completely deleted or replaced in the ieLANA1 polypeptide.

12. The method of claim 2, wherein the LANA1 amino acid sequence comprises the amino acid sequence of SEQ ID NO: 1 in which from amino acid 429 to amino acid 938 are deleted.

13. The method of claim 12, wherein at least about 50% of both CR2 and CR3 are deleted from the LANA1 amino acid sequence.

14. The method of claim 12, wherein at least about 75% of both CR2 and CR3 are deleted from the LANA1 amino acid sequence.

15. The method of claim 12, wherein at least about 95% of both CR2 and CR3 are deleted from the LANA1 amino acid sequence.

16. The method of claim 2, wherein the LANA1 amino acid sequence comprises the amino acid sequence of SEQ ID NO: 1 in which from amino acid 429 to amino acid 775 are deleted.

17. The method of claim 2, wherein the LANA1 amino acid sequence comprises the amino acid sequence of SEQ ID NO: 1 in which from amino acid 442 to amino acid 775 are deleted.

18. The method of claim 1, comprising obtaining a cell from the patient, transforming the cell with a nucleic acid capable of expressing the ieLANA1 polypeptide in the cell, and transferring the transformed cell back into the patient thereby eliciting the immune response.

19. The method of claim 18, wherein the cell is obtained from Peripheral Blood Lymphocytes.

20. The method of claim 18, wherein the cell is a dendritic cell.

21. The method of claim 1, wherein the ieLANA1 polypeptide is administered to the patient parenterally in a pharmaceutically-acceptable carrier.

22. The method of claim 21, wherein the ieLANA1 polypeptide is administered with an adjuvant.

23. The method of claim 1, comprising transferring a nucleic acid comprising a gene into a cell of the patient, wherein the gene encodes and expresses the ieLANA1 polypeptide.

24. The method of claim 23, wherein the nucleic acid is transferred parenterally to the patient.

25. The method of claim 23, wherein the nucleic acid is contained in a composition comprising a pharmaceutically-acceptable carrier.

26. The method of claim 23, wherein the nucleic acid is transferred to the cell of the patient ex vivo.

27. The method of claim 1, wherein the ieLANA1 polypeptide comprises the LANA1 amino acid sequence comprising one or more LANA1 T-cell epitopes and a protein destabilization domain and the ieLANA1 polypeptide exhibits increased proteasomal degradation as compared to wild-type LANA1 protein.

28. The method of claim 27, wherein the protein destabilization domain is one of a D-Box, KEN, PEST, Cyclin A and UFD domain/substrate.

29. An immunologically-enhanced LANA1 polypeptide (“ieLANA1 polypeptide”) for eliciting a CTL immune response to LANA1, comprising a LANA1 amino acid sequence comprising one or more LANA1 T-cell epitopes, wherein the LANA1 amino acid sequence is modified to exhibit increased proteasomal degradation as compared to wild-type LANA1 protein.

30. The ieLANA1 polypeptide of claim 29, wherein the ieLANA1 polypeptide does not comprise a portion of a LANA1 central repeat domain having the capacity to inhibit proteasomal degradation of a polypeptide or inhibit translation of a polypeptide attached in frame to that portion of the LANA1 central repeat domain, so that the ieLANA1 polypeptide exhibits increased proteasomal degradation as compared to wild-type LANA1 protein.

31. The ieLANA1 polypeptide of claim 30, wherein the LANA1 amino acid sequence comprises the amino acid sequence of SEQ ID NO: 1 in which a portion of the LANA1 central repeat domain having the capacity to inhibit proteasomal degradation of a polypeptide and inhibit translation of a polypeptide is modified to decrease the ability of that portion to inhibit proteasomal degradation of a polypeptide.

32. The ieLANA1 polypeptide of claim 31, wherein the LANA1 amino acid sequence comprises the amino acid sequence of SEQ ID NO: 1 in which from about amino acid 330 to about amino acid 938 are deleted.

33. The ieLANA1 polypeptide of claim 32, wherein the LANA1 amino acid sequence comprises the amino acid sequence of SEQ ID NO: 1 in which from amino acid 330 to amino acid 938 are deleted.

34. The ieLANA1 polypeptide of claim 30, wherein a portion of one or both of CR2 and CR3 of the LANA1 amino acid sequence is modified in the ieLANA1 polypeptide to decrease the ability of that portion of one or both of CR2 and CR3 to inhibit proteasomal degradation of the ieLANA1 polypeptide.

35. The ieLANA1 polypeptide of claim 34, wherein the LANA1 amino acid sequence is modified such that one or both of CR2 and CR3 are substantially or completely deleted.

36. The ieLANA1 polypeptide of claim 34, wherein the LANA1 amino acid sequence comprises the amino acid sequence of SEQ ID NO: 1 in which from about 20 to 98 amino acids of amino acids 428-938 are deleted.

37. The ieLANA1 polypeptide of claim 34, in which CR2 is substantially or completely deleted or replaced in the ieLANA1 polypeptide.

38. The ieLANA1 polypeptide of claim 34, in which CR3 is substantially or completely deleted or replaced in the ieLANA1 polypeptide.

39. The ieLANA1 polypeptide of claim 34, in which CR2 and CR3 are substantially or completely deleted or replaced in the ieLANA1 polypeptide.

40. The ieLANA1 polypeptide of claim 30, wherein the LANA1 amino acid sequence comprises the amino acid sequence of SEQ ID NO: 1 in which from amino acid 429 to amino acid 938 are deleted.

41. The ieLANA1 polypeptide of claim 30, wherein at least about 50% of both CR2 and CR3 are deleted from the LANA1 amino acid sequence.

42. The ieLANA1 polypeptide of claim 30, wherein at least about 75% of both CR2 and CR3 are deleted from the LANA1 amino acid sequence.

43. The ieLANA1 polypeptide of claim 30, wherein at least about 95% of both CR2 and CR3 are deleted from the LANA1 amino acid sequence.

44. The ieLANA1 polypeptide of claim 30, wherein the LANA1 amino acid sequence comprises the amino acid sequence of SEQ ID NO: 1 in which from amino acid 429 to amino acid 775 are deleted.

45. The ieLANA1 polypeptide of claim 30, wherein the LANA1 amino acid sequence comprises the amino acid sequence of SEQ ID NO: 1 in which from amino acid 442 to amino acid 775 are deleted.

46. The ieLANA1 polypeptide of claim 29, wherein the ieLANA1 polypeptide comprises the LANA1 amino acid sequence comprising one or more LANA1 T-cell epitopes and a protein destabilization domain and the ieLANA1 polypeptide exhibits increased proteasomal degradation as compared to wild-type LANA1 protein.

47. The ieLANA1 polypeptide of claim 46, wherein the protein destabilization domain is one of a D-Box, KEN, PEST, Cyclin A and UFD domain/substrate.

48. The ieLANA1 polypeptide of claim 29, contained within a composition further comprising a pharmaceutically-acceptable carrier.

49. The ieLANA1 polypeptide of claim 29, contained within a composition further comprising an adjuvant.

50. An isolated nucleic acid comprising an open reading frame encoding an immunologically-enhanced LANA1 polypeptide for eliciting a CTL immune response to LANA1 as claimed in claim 29.

51. A polypeptide other than full-length LANA1 protein, comprising at the N-terminal or C-terminal end of the polypeptide, or inserted within the polypeptide an amino acid sequence obtained from or derived from one of both of a CR2 and CR3 region of a LANA1 central repeat domain and having the capacity to inhibit proteasomal degradation of a polypeptide or inhibit translation of the polypeptide as compared to the same polypeptide without the amino acid sequence.

52. The polypeptide of claim 51, wherein the polypeptide comprises from about amino acid 330 to from about amino acid 938 of SEQ ID NO: 1.

53. The polypeptide of claim 52, wherein the polypeptide comprises 50 or more consecutive amino acids of amino acids 434-938 of SEQ ID NO: 1.

54. The polypeptide of claim 51, comprising from about 50 to 509 consecutive amino acids of amino acids 434-938 of SEQ ID NO: 1.

55. The polypeptide of claim 54, wherein the 50 to 509 amino acids comprise a junction between the CR2 and CR3 regions.

56. The polypeptide of claim 51, wherein the polypeptide comprises from amino acid 429 to amino acid 775 of SEQ ID NO: 1.

57. The polypeptide of claim 51, wherein the polypeptide comprises from amino acid 442 to amino acid 775 of SEQ ID NO: 1.

58. The polypeptide of claim 51, comprising a derivative of the portion of the LANA1 central repeat domain having five or more iterations of one or both of the amino acid sequences QQQDE (SEQ ID NO: 3) and QQQEP (SEQ ID NO: 4).

59. The polypeptide of claim 51, wherein the polypeptide comprises from 50 to 100 consecutive amino acids comprising at least about 50% Q residues.

60. The polypeptide of claim 59, wherein the polypeptide comprises five or more iterations of the amino acid sequence QQQ motifs separated by one or two amino acids.

61. The polypeptide of claim 60, wherein the one or two amino acids are selected from D, E and P.

62. The polypeptide of claim 51, wherein the polypeptide further comprises an amino acid sequence comprising five or more iterations of one of the amino acid motif QELEE (SEQ ID NO: 5) attached to the C-terminal end of the portion of the polypeptide comprising from 50 to 100 consecutive amino acids comprising at least about 50% Q residues.

63. The polypeptide of claim 51, further comprising at least about 25 consecutive amino acids from a GA repeat region of an EBV EBNA1 protein.

64. The polypeptide of claim 51, comprising from about 50 to about 148 consecutive amino acids of amino acids 768 to 916 of SEQ ID NO: 1 connected to the C-terminus of from about 50 to about 340 consecutive amino acids of amino acids 428 to 768 of SEQ ID NO: 1.

65. A method of inhibiting proteasomal degradation of a polypeptide comprising attaching to the N-terminal or C-terminal end of the polypeptide an amino acid sequence obtained from or derived from one or both of a CR2 and CR3 region of a LANA1 central repeat domain and having the capacity to inhibit proteasomal degradation of a polypeptide and inhibit translation of the polypeptide as compared to the same polypeptide without the amino acid sequence.

66. The method of claim 60, wherein the amino acid sequence is obtained from or derived from both of the CR2 and CR3 region of the LANA1 central repeat domain.

67. The method of claim 66, wherein the polypeptide comprises amino acids 330-938 of SEQ ID NO: 1.

68. The method of claim 66, wherein the polypeptide comprises at least about 100 consecutive amino acids of amino acids 330-938 of SEQ ID NO: 1.

69. The method of claim 66, wherein the polypeptide comprises amino acids 429-775 of SEQ ID NO: 1.

70. The method of claim 66, wherein the polypeptide comprises amino acids 442-775 of SEQ ID NO: 1.

71. The method of claim 66, wherein the polypeptide comprises a derivative of the portion of a LANA1 central repeat domain having five or more iterations of one or both of the amino acid sequences QQQDE (SEQ ID NO: 3) and QQQEP (SEQ ID NO: 4).

72. The method of claim 66, wherein the polypeptide comprises from 50 to 100 consecutive amino acids comprising at least about 50% Q residues.

73. The method of claim 72, wherein the polypeptide comprises five or more iterations of the amino acid sequence QQQ motifs separated by one or two amino acids.

74. The method of claim 73, wherein the one or two amino acids are selected from D, E and P.

75. The polypeptide of claim 73, wherein the polypeptide further comprises an amino acid sequence comprising five or more iterations of the amino acid motif QELEE (SEQ ID NO: 5) attached to the C-terminal end of the portion of the polypeptide comprising from 50 to 100 consecutive amino acids comprising at least about 50% Q residues.

76. The method of claim 65, the polypeptide further comprising at least about 25 consecutive amino acids from a GA repeat region of an EBV EBNA1 protein.

77. The method of claim 65, the polypeptide comprising from about 50 to about 148 consecutive amino acids of amino acids 768 to 916 of SEQ ID NO: 1 connected to the C-terminus of from about 50 to about 340 consecutive amino acids of amino acids 428 to 768 of SEQ ID NO: 1.

78. An isolated nucleic acid comprising an open reading frame encoding a polypeptide other than full-length LANA1 protein, comprising at the N-terminal or C-terminal end of the polypeptide, or inserted within the polypeptide an amino acid sequence obtained from or derived from one of both of a CR2 and CR3 region of a LANA1 central repeat domain and having the capacity to inhibit proteasomal degradation of a polypeptide or inhibit translation of the polypeptide as compared to the same polypeptide without the amino acid sequence.

79. The nucleic acid of claim 78, wherein the nucleic acid comprises a gene for expressing the open reading frame.

80. The nucleic acid of claim 79, wherein the gene is contained in a vector.

81. The nucleic acid of claim 80, wherein the vector is a viral vector.

82. A method of eliciting an immune response to a protein comprising a synthesis retardation and proteasome degradation inhibition domain in a patient comprising introducing into a cell of the patient an immunologically-enhanced version of the protein for eliciting a CTL immune response to the protein, comprising an amino acid sequence of the protein comprising one or more T-cell epitopes of the protein, and wherein the amino acid sequence of the protein is modified to exhibit increased proteasomal degradation as compared to a wild-type version of the protein.

83. The method of claim 82, wherein the immunologically-enhanced version of the protein does not comprise a portion of a domain having the capacity to inhibit proteasomal degradation of a polypeptide or inhibit translation of a polypeptide attached in frame to that portion of the protein, so that the immunologically-enhanced version of the protein exhibits increased proteasomal degradation as compared to wild-type protein.

84. The method of claim 82, wherein the protein is a gammaherpesvirus latency protein.

85. The method of claim 82, wherein the protein is EBV EBNA1.

86. The method of claim 82, wherein the immunologically-enhanced version of the protein comprises an amino acid sequence comprising one or more one or more T-cell epitopes of the protein and a protein destabilization domain and the immunologically-enhanced version of the protein exhibits increased proteasomal degradation as compared to a wild-type version of the protein.

87. The method of claim 86, wherein the protein destabilization domain is one of a D-Box, KEN, PEST, Cyclin A and UFD domain/substrate.

88. A polypeptide comprising an immunologically-enhanced version of a protein comprising a synthesis retardation and proteasome degradation inhibition domain for eliciting a CTL immune response to the protein, comprising an amino acid sequence of the protein comprising one or more T-cell epitopes of the protein, and wherein the amino acid sequence of the protein is modified to exhibit increased proteasomal degradation as compared to a wild-type version of the protein.

89. The polypeptide of claim 88, wherein the immunologically-enhanced version of the protein does not comprise a portion of a domain having the capacity to inhibit proteasomal degradation of a polypeptide or inhibit translation of a polypeptide attached in frame to that portion of the protein, so that the immunologically-enhanced version of the protein exhibits increased proteasomal degradation as compared to wild-type protein.

90. The polypeptide of claim 88, wherein the protein is a gammaherpesvirus latency protein.

91. The polypeptide of claim 88, wherein the protein is EBV EBNA1.

92. The polypeptide of claim 88, wherein the immunologically-enhanced version of the protein comprises an amino acid sequence comprising one or more one or more T-cell epitopes of the protein and a protein destabilization domain and the immunologically-enhanced version of the protein exhibits increased proteasomal degradation as compared to a wild-type version of the protein.

93. The polypeptide of claim 88, wherein the protein destabilization domain is one of a D-Box, KEN, PEST, Cyclin A and UFD domain/substrate.

94. An isolated nucleic acid comprising an open reading frame encoding the polypeptide of claim 88.
